(19) United States
(12) Patent Application Publication
(10) Pub. No.: US 2006/0240500 A1
(43) Pub. Date: Oct. 26, 2006

(54) Diagnosis of kidney damage and protection against same

(75) Inventors: John J. Kopchick, Athens, OH (US); Karen T. Coschigano, The Plains, OH (US); Amy N. Wetzel, Wooster, OH (US)

Correspondence Address: BROWDY AND NEIMARK, P.L.L.C.;624 NINTH STREET, NW, SUITE 300, WASHINGTON, DC, 20001-5303, US

(73) Assignee: Ohio University, Athens, OH (US)

(21) Appl. No.: 10/524,237

(22) PCT Filed: Aug. 4, 2003
(86) PCT No.: PCT/US03/24053

§ 371 Date: Mar. 29, 2006

Related U.S. Application Data
(60) Provisional application No. 60/400,052, filed on Aug. 2, 2002.

(51) Int. Cl. A61K 48/00; A61K 38/54; C12Q 1/62

(52) U.S. Cl. 435/10; 514/12; 514/44

(57) ABSTRACT

Various nucleic acids and proteins have been identified by differential hybridization methods as useful as markers for diagnosing kidney damage. The identified marker proteins include (I) androgen related protein, SON protein, FUSE binding Protein 1, claudin10, heat shock protein, phospho triesterase related protein, ubiquitin protein ligase Nedd-4, and Ac39/physophilin, and (II) disabled-2 p96, palmitylated serine/threonine kinase, tumor differentially expressed 1 protein, cytochrome oxidase III, TLH 39 protein precursor, hydroxysteroid dehydrogenase 4 delta <5>-3 beta, and glutathione peroxidase III. The proteins of group (I), and antagonists of the proteins of group (II), are useful for protecting mammals against kidney damage.

### BACKGROUND OF THE INVENTION

1. Field of the Invention

The invention relates to various nucleic acid molecules and proteins, and their use in (1) diagnosing kidney damage, or conditions associated with the development of kidney damage, and (2) protecting mammals (including humans) against kidney damage.

2. Description of the Background Art

Kidney Anatomy and Physiology

In humans, the kidneys are the main excretory organs, eliminating urea, citric acid, creatinine and other waste metabolites. The kidneys also conserve or excrete water and electrolytes as required. Finally, they produce the hormone erythropoietin as well as other proteins.

The kidneys are paired organs situated on the posterior wall of the adrenal cavity. The cortex is the outer layer. The medulla consists of multiple triangular renal pyramids whose bases are covered by the cortex and whose tips (papillae) project into the minor calyces of the renal pelvis. Within the renal pelvis, the minor calyces join to form a major calyx, and the major calyces join to form the renal pelvis, the expanded upper end of the ureter.

The functional unit of the kidney, where urine is formed, is the nephron. Each nephron consists of (1) a knot of coiled capillaries called a glomerulus, and (2) a tubule. The proximal end of the tubule forms a cup, the glomerular capsule, which surrounds the glomerulus. The glomerular capsule and glomerulus together form the renal corpuscle. The renal corpuscles are located in the cortical region of the kidney. Moving away from the glomerular capsule, the nephron has, in order (1) the proximal convoluted tubule, (2) the loop of Henle, and (3) the distal convoluted tubule.

The distal convoluted tubules of several nephrons empty into a collecting tubule, and 10-25 collecting tubules empty into each renal pyramid.

Blood passes from the aorta to the renal artery, which divides into interlobar arteries (in the renal columns of the cortex, which separate the pyramids), then into arcuate arteries parallel to the surface of the kidney, then into interlobular arteries, and then into afferent arterioles, each of which supplies a renal corpuscle and forms a capillary network called a glomerulus. The blood leaves through an efferent arteriole that divides into peritubular capillaries surrounding the tubules; these capillaries converge into interlobular veins, which feed arcuate veins, which empty into interlobar veins, and then connect to the renal vein, which in turn feeds the inferior vena cava. There thus are two capillary beds, the glomerulus and the peritubular capillaries.

The glomeruli and the renal tubules (nephrons and collecting tubules) participate in glomerular filtration, tubular reabsorption, and tubular secretion.

Glomerulosclerosis

Glomerulosclerosis refers to a scarring of the glomeruli. The “scarring” is caused by deposition of cellular basement membrane proteins such as collagen IV. The scarring disrupts the filtering process of the kidneys, allowing protein to leak from the blood into the urine (proteinurea). About 15% of people with proteinurea are diagnosed, usually by kidney biopsy, as having glomerulosclerosis. The scarred glomeruli cannot be repaired.

End Stage Renal Disease

Many patients with glomerulosclerosis gradually get worse until their kidneys fail completely, a condition known as end stage renal disease (ESRD). Patients with ESRD must go on dialysis or receive a kidney transplant (a new kidney). The progression of kidney failure can be slowed using ACE inhibitors.

Each year, in the United States, nearly 80,000 people suffer kidney failure. In 1998, the causes of kidney failure were diabetes (43.2%), high blood pressure (23.0%), glomerulonephritis (12.3%), polycystic kidney disease (2.9%), or other (18.6%).

Diabetes

Diabetes mellitus is a pleiotropic disease of great complexity. The two major types have been termed type I or insulin-dependent diabetes mellitus (IDDM) and type II or non-insulin-dependent diabetes mellitus (NIDDM). Type II diabetes is the predominant form found in the Western world; fewer than 8% of diabetic Americans have the type I disease. Development of type II diabetes may be linked to a more sedentary lifestyle and obesity.

Type I diabetics are often characterized by their low or absent levels of circulating endogenous insulin (Unger and Foster, 1998). Islet cell antibodies causing damage to the pancreas are frequently present at diagnosis. Injection of exogenous insulin is required to prevent ketosis and sustain life. Type II diabetics are often characterized by hyperinsulinemia and an increasing resistance to insulin. They are usually not insulin dependent or prone to ketosis under normal circumstances. Hyperglycemia can usually be controlled by an alteration in diet and amount of exercise, but insulin treatment may be required.

Complications of diabetes (end organ damage) include retinopathy, neuropathy, and nephropathy (traditionally designated as microvascular complications) as well as atherosclerosis (a macrovascular complication). It has been shown that meticulous blood glucose control can often slow down or halt the progression of diabetic complications if caught early enough (Unger and Foster, 1998). However, tight metabolic control is extremely difficult to achieve.

Diabetic Nephropathy

Diabetic nephropathy is the leading cause of end stage renal disease. About 20 million people in the United States have diabetes, and about 100,000 people have kidney failure as a result of diabetes.

Initial nephromegaly as well as glomerular hypertrophy and microalbuminuria, once thought to be limited to type I diabetics, are now seen equally in type II diabetics (Wirta et al., 1996). The pathology of diabetic nephropathy begins with thickening of the glomerular basement membrane (GBM), an increase in mesangial matrix, and subintimal hyaline thickening of both afferent and efferent arterioles (Unger and Foster, 1998). The capacity to clear macromolecules is impaired in diabetes, resulting in accumulation of albumin and larger proteins within the glomerular wall and in the mesangium that may lead to stimulation of mesangial matrix production.

The progression of kidney failure occurs in five stages (Wardle, 1996): (I) hyperfiltration (increased blood flow) and enlargement of the kidneys (II) glomeruli begin to show damage, e.g., by microalbuminuria (20-200 μg/min loss of albumin); (III) dipstick-positive proteinurea (>200 μg/min), blood levels of creatinine and urea-nitrogen use, (IV) glomerular filtration decreases to less than 75 ml/min, and (V) kidney failure: filtration rate less than 10 ml/min.

Features of the first stage include an increase in kidney volume (renal hypertrophy) and function (increased glomerular filtration rate or GFR). Features of the second stage include a thickened GBM. Features of the third stage include detection of microalbuminuria. Once this stage is reached, there is little chance for reversal and the disease progresses on to the fourth stage (glomerulosclerosis and macroalbuminuria) and the fifth stage (ESRD).

Other Kidney Disorders

Glomerulonephritis is an inflammation of the glomeruli, often due to an allergic reaction to streptococcal toxins.

Pyelonephritis is a bacterial infection of the kidney pelvis and surrounding tissues.

Kidney stones may be formed in the renal pelvis.

Growth Hormone

Growth hormone has many roles, ranging from regulation of protein, fat and carbohydrate metabolism to growth promotion. GH is produced in the somatrophic cells of the anterior pituitary and exerts its effects either through the GH-induced action of IGF-I, in the case of growth promotion, or by direct interaction with the GHR on target cells including liver, muscle, adipose, and kidney cells. Hyposecretion of GH during development leads to dwarfism, and hypersecretion before puberty leads to gigantism. In adults, hypersecretion of GH results in acromegaly, a clinical condition characterized by enlarged facial bones, hands, feet, fatigue and an increase in weight. Of those individuals with acromegaly, 25% develop type 2 diabetes. This may be due to insulin resistance caused by the high circulating levels of GH leading to high circulating levels of insulin (Kopchick et al., Annual Rev. Nutrition 1999. 19:437-61).

A further mode of GH action may be through the transcriptional regulation of a number of genes contributing to the physiological effects of GH.

Diabetic Glomerulosclerosis in Animal Models

One hallmark of kidney damage is the development of glomerulosclerosis. Several mouse models develop glomerulosclerosis similar to that seen in human diabetics. Streptozotocin (STZ) induced diabetic mice, as well as the spontaneously occurring non-obese diabetic mice, develop glomerulosclerosis.

Interestingly, bovine growth hormone (bGH) expressing transgenic mice, which are not diabetic but are hyperinsulinemic, develop severe glomerulosclerosis. In contrast, both bGH antagonist transgene mice and GH receptor/binding protein (GHR/BP) gene-disrupted homozygous (−/−) mice are protected from kidney damage, even when diabetes is induced by STZ treatment.

McGrane, et al., J. Biol. Chem. 263:11443-51 (1988) and Chen, et al., J. Biol. Chem., 269:15892-7 (1994) describe the genetic engineering of mice to express bovine growth hormone (bGH) or human growth hormone (hGH), respectively. These mice exhibited an enhanced growth phenotype. They also developed kidney lesions similar to those seen in diabetic glomerulosclerosis, see Yang, et al., Lab. Invest., 68:62-70 (1993). Ogueta, et al., J. Endocrinol., 165: 321-8 (2000) reported that transgenic mice expressing bovine GH develop arthritic disorder and self-antibodies.

Growth hormone genes and the proteins encoded by them can be converted into growth hormone antagonists by mutation, see Kopchick U.S. Pat. No. 5,350,836. Transgenic mice have been made that express the GH antagonists bGH-G119R or hGH G120R, and which exhibit a dwarf phenotype. Chen, et al., J. Biol. Chem., 263:15892-7 (1994); Chen, et al., Mol. Endocrinol, 5:1845-52 (1991); Chen, et al., Proc. Nat. Acad. Sci. USA 87:5061-5 (1990). These mice did not develop kidney lesions. See Yang (1993), supra.

Chen, et al., Endocrinol, 136:660-7 (1995) compared the effect of streptozotocin treatment in normal nontransgenic mice, and in mice transgenic for (1) a GH receptor antagonist, the G119R mutant of bovine growth hormone or (2) the E117L-mutant of bGH. (According to Chen's ref. 24, these large GH transgenic streptozotocin-treated mice constitute an animal model for diabetes.) Glomerulosclerosis was seen in diabetic (STZ-treated) nontransgenic mice and in diabetic bGH-E117L mice, but not in diabetic bGH-G119R mice.

Two of the proteins which mediate growth hormone activity are the growth hormone receptor and the growth hormone binding protein, encoded by the same gene in mice (GHR/BP). It is possible to genetically engineer mice so that the gene encoding these proteins is disrupted (“knocked-out”; inactivated), see Zhou, et al., Proc. Nat. Acad. Sci. (USA), 94:13215-20 (1997). Zhou, et al. inactivated the GHR/BP gene by replacing the 3′ portion of exon 4 (which encodes a portion of the GH binding domains) and the 5′ region of intron 4 with a neo gene cassette. The modified gene was introduced into the target mice by homologous recombination. Like mice expressing a GH antagonist, homozygous GHR/BP-KO mice exhibit a dwarf phenotype. GHR/BP-KO mice, made diabetic by streptozotocin treatment, are protected from the development of diabetes-associated nephropathy. Bellush, et al., Endocrinol., 141:163-8 (2000).

Differential/Subtractive Hybridization

Zhang, et al., Kidney International, 56:549-558 (1999) identified genes up-regulated in 5/6 nephrectomized (subtotal renal ablation) mouse kidney by a PCR-based subtraction method. Ten known and nine novel genes were identified. The ultimate goal was to identify genes involved in glomerular hyperfiltration and hypertrophy.

Melia, et al., Endocrinol., 139:688-95 (1998) applied subtractive hybridization methods for the identification of androgen-regulated genes in mouse kidney. The treatment mice were dosed with dihydrotestosterone, an androgen. Kidney androgen-regulated protein gene was used as a positive control, as it is known to be up-regulated by DHT.

Holland, et al., Abstract 607, “Identification of Genes Possibly Involved in Nephropathy of Bovine Growth Hormone Transgenic Mice” (Endocrine Society Meeting, Jun. 22, 2000) describes the screening strategy of Example 3, but does not disclose the identity of any of the differentially expressed clones.

Coschigano, et al., Abstract 333, “Identification of Genes Potentially Involved in Kidney Protection During Diabetes” (Endocrine Society Meeting, Jun. 22, 2000) describes the screening strategy of Example 1, but does not disclose the identity of any of the differentially expressed clones.

The following differential hybridization articles may also be of interest:
Wada, et al., “Gene expression profile in streptozotocin-induced diabetic mice kidneys undergoing glomerulosclerosis”, Kidney Int, 59:1363-73 (2001);
Song, et al., “Cloning of a novel gene in the human kidney homologous to rat munc13S: its potential role in diabetic nephropathy”, Kidney Int., 53:1689-95 (1998);
Page, et al., “Isolation of diabetes-associated kidney genes using differential display”, Biochem. Biophys. Res. Comm., 232:49-53 (1997).
Peradi, “Subtractive hybridization claims: An efficient technique to detect overexpressed mRNAs in diabetic nephropathy,” Kidney Int. 53:926-31 (1998).
Condorelli, EMBO J., 17:3858-66 (1998).
See also WO00/66784 (differential hybridization screening for brown adipose tissue); PCT/US00/12366, filed May 5, 2000 (differential hybridization screening for liver).
   Identification of Genes Involved in Nephropathy

Attention recently has focused on the generation of kidney mRNA expression profiles and the identification of genes involved in nephropathy. Initial studies examined expression in the kidney of known genes, such as α1IV collagen, laminin B1, TGF-β1, 72 kDa collagenase, and TIMP-3, which are involved in extracellular matrix synthesis and degradation (Esposito et al., 1996; Striker et al., 1996; Yang et al., 1997; Yang et al., 1994). Changes in facilitative glucose transporter, receptors for advanced glycation end products (RAGE), and TGFβ isoform and receptor messages have also been examined (Chin et al., 1997; Hill et al., 2000; Ziyadeh et al., 1997). With recent advances in gene profiling techniques, several databases documenting mRNA expression in the kidney as a whole as well as in individual cell types are being generated (Takenaka et al., 1998; Virlon et al., 1999; Wada et al., 2001). These techniques, as well as others, including differential display and subtractive hybridization, are being used to identify additional genes involved in the development and progression of diabetic nephropathy (Condorelli et al., 1998; Page et al., 1997; Peraldi et al., 1998; Song et al., 1998; Wada et al., 2001).

### SUMMARY OF THE INVENTION

Differential hybridization techniques have been used to identify mouse genes that are differentially expressed in mice, depending upon the extent or susceptibility to kidney damage. By identifying related human genes and proteins, one may identify agents useful in protecting humans against kidney damage. Protection against kidney damage mediated at least in part by growth hormone, and/or associated with diabetes, is of particular interest.

Based on the differential expression of clones H8, H1, F1, F2, F4, F5, F6, F27, F39, G38, G16, F16, B45, B3 and B46, we believe that the human proteins most closely corresponding to mouse vacuolar adenosine triphosphate, ubiquitin protein ligase Nedd-4, SON protein, FUSE binding protein 1, kidney androgen related protein, claudin 10, and heat shock protein 105 kDa (Hsp 105), a phosphotriesterase, certain immunoglobulins, and certain other proteins identified below, have a protective effect.

Similarly, based on the differential expression of clones G26, C22, A8, A34, A39, A48, E39, F21, F38, F40, G9, G24 and G28, we believe that it would be desirable to inhibit the expression of the human proteins most closely corresponding to mouse 3-beta-hydroxysteroid dehydrogenase type 4, disable-2 p96 (Dab2), palmitylated serine/threonine kinase, tumor differentially expressed 1 (TDE1), cytochrome oxidase III, ERG2 protein and/or glutathione peroxidase 3, certain immunoglobulins, and certain other proteins identified below, or to neutralize their activity, with a suitable antagonist (e.g., a binding antibody or oligopeptide).

Thus, Applicants contemplate:

(1) use of the “favorable” mouse DNAs of the Master Table (below) to isolate or identify related human DNAs;

(2) use of human DNAs, related to favorable mouse DNAs, to express the corresponding human proteins;

(3) use of the corresponding human proteins (and mouse proteins, if the sequence is sufficiently complete to be biologically active, and is active in humans), to protect against kidney damage; and

(4) use of the corresponding mouse or human proteins, in diagnostic agents, in assays to measure kidney damage or protection against such damage.

Moreover Applicants contemplate:

(1) use of the “unfavorable” mouse DNAs of the Master Table to isolate or identify related human DNAs;

(2) use of the complement to the “unfavorable” mouse DNAs or related human DNAs, as antisense molecules to inhibit expression of the related human DNAs;

(3) use of the mouse or human DNAs to express the corresponding mouse or human proteins;

(4) use of the corresponding mouse or human proteins, in diagnostic agents, in assays to measure kidney damage;

(5) use of the corresponding mouse or human proteins in assays to determine whether a substance binds to (and hence may neutralize) the protein; and

(6) use of the neutralizing substance to protect against kidney damage.

The related human DNAs may be identified by comparing the mouse sequence (or its AA translation product) to known human DNAs (and their AA translation products). If this is unsuccessful, human cDNA or genomic DNA libraries may be screened using the mouse DNA as a probe.

Agents which bind the “favorable” and “unfavorable” nucleic acids, or the corresponding proteins (e.g., an antibody vs. the protein) may be used to screen for kidney damage.

### BRIEF DESCRIPTION OF THE DRAWINGS

FIG. 1. Changes in glomerular volume (left panel) and urinary albumin excretion (right panel) as a function of duration of STZ-induced diabetes in wild-type mice. Glomerular measurements were made from sections of kidney fixed in 4% paraformaldehyde (FIG. 1A). Urinary albumin excretion was determined by radioimmune assay (RIA) using a rat albumin antibody. Urinary volume output was normalized by creatinine concentration comparisons using a standard colorimetric assay (FIG. 1B). ND: nondiabetic; DB, diabetic.

FIG. 2. Physiological assessment of the progression of nephropathy in the kidney of NT and bGH female mice at 2, 5, and 12 months of age. Glomerular (FIG. 2A) and mesangial (FIG. 2B) measurements were made from sections of kidney fixed in 4% paraformaldehyde. Urinary albumin excretion (FIG. 2C) was determined by radioimmune assay (RIA) using a rat albumin antibody. Urinary volume output was normalized by creatinine concentration comparisons using a standard calorimetric assay.

### DETAILED DESCRIPTION OF THE PREFERRED EMBODIMENTS OF THE INVENTION

Identified Differentially Expressed cDNAs

In three differential expression studies (examples 1-3), we have obtained cDNAs which were differentially expressed.

In Example 1, we looked at differential expression of genes in kidneys of streptozotocin-treated mice (a model of diabetes) as a result of GHR/BP knockout. The clones of interest included: G26, H8, H1, F1, G38, G9, G16, G24, G28 (favorables underlined).

In Example 2, we obtained data on differential expression of genes in kidneys of nontransgenic mice as a result of streptozotocin treatment. The clones of interest included: F1, F4, F5, F6, F27, E39, F2, F16, F21, F38, F39, and F40.

In Example 3, we studied differential expression of genes in kidneys of mice as a result of overproduction of growth hormone. The clones of interest included: A8, A34, A39, A48, B45, C22, B3, B46.

Direct and Indirect Utility of Identified Nucleic Acid Sequences and Related Molecules

The cDNAs of the disclosed clones may be used directly. For diagnostic or screening purposes, they (or specific binding fragments thereof) may be labeled and used as hybridization probes. For therapeutic purposes, they (or specific binding fragments thereof) may be used as antisense reagents to inhibit the expression of the corresponding gene, or of a sufficiently homologous gene of another species.

If the cDNA appears to be a full-length cDNA, that is, that it encodes an entire, functional protein, then it may be used in the expression of that protein. Such expression may be in cell culture, with the protein subsequently isolated and administered exogenously to subjects who would benefit therefrom, or in vivo, i.e., administration by gene therapy. Naturally, any DNA encoding the same protein, or a fragment or a mutant protein which retains the desired activity, may be used for the same purpose. The encoded protein of course has utility therapeutically and, in labeled or immobilized form, diagnostically.

The cDNAs of the disclosed clones may also be used indirectly, that is, to identify other useful DNAs, proteins, or other molecules. We have attempted to determine whether the cDNAs disclosed herein have significant similarity to any known DNA, and whether, in any of the six possible combinations of reference frame and strand, they encode a protein similar to a known protein. If so, then it follows that the known protein, and DNAs encoding that protein, may be used in a similar manner. In addition, if the known protein is known to have additional homologues, then those homologous proteins, and DNAs encoding them, may be used in a similar manner.

There thus are several ways that a human protein homologue of interest can be identified by database searching, including:

1) a DNA->DNA (BlastN) search for database DNAs closely related to the mouse cDNA clone identifies a particular mouse (or other nonhuman, e.g., rat) gene, and that nonhuman gene encodes a protein for which there is a known human protein homologue;

2) a DNA->Protein (BlastX) search for database proteins closely related to the translated DNA of the mouse cDNA clone identifies a particular mouse (or other nonhuman) protein, and that nonhuman protein has a known human protein homologue;

3) a DNA->DNA (BlastN) search of the database for human DNAs closely related to the mouse cDNA clone identifies a particular human DNA as a homologue of the mouse cDNA, and the corresponding human protein is known (e.g., by translation of the human DNA); and

4) a DNA->Protein (BlastX) search of the database for human proteins closely related to the translated DNA of the mouse cDNA clone identifies a particular human protein as a homologue of the corresponding mouse protein.

Thus, if we have identified a mouse cDNA, and it encodes a mouse protein which appears similar to a human protein, then that human protein may be used (especially in humans) for purposes analogous to the proposed use of the mouse protein in mice. Moreover, a specific binding fragment of an appropriate strand of the corresponding human gene or cDNA could be labeled and used as a hybridization probe (especially against samples of human mRNA or cDNA).

In determining whether the disclosed cDNAs have significant similarities to known DNAs (and their translated AA sequences to known proteins), one would generally use the disclosed cDNA as a query sequence in a search of a sequence database. The results of several such searches are set forth in the Examples. Such results are dependent, to some degree, on the search parameters. Preferred parameters are set forth in Example 1. The results are also dependent on the content of the database. While the raw similarity score of a particular target (database) sequence will not vary with content (as long as it remains in the database), its informational value (in bits), expected value, and relative ranking can change. Generally speaking, the changes are small.

It is possible to use the sequence of the entire cDNA insert to query the database. However, the error rate increases as a sequencing run progresses. Hence, it may be beneficial to search the database using a truncated (presumably more accurate) sequence, especially if the insert is quite long.

It will be appreciated that the nucleic acid and protein databases keep growing. Hence a later search may identify high scoring target sequences which were not uncovered by an earlier search because the target sequences were not previously part of a database.

Hence, in a preferred embodiment, the cognate DNAs and proteins include not only those set forth in the examples, but those which would have been highly ranked (top ten, more preferably top three, even more preferably top two, most preferably the top one) in a search run with the same parameters on the date of filing of this application.

If the cDNA appears to be a partial cDNA, it may be used as a hybridization probe to isolate the full-length cDNA. If the partial cDNA encodes a biologically functional fragment of the cognate protein, it may be used in a manner, similar to the full length cDNA, i.e., to produce the functional fragment.

If we have indicated that an antagonist of a protein or other molecule is useful, then such an antagonist may be obtained by preparing a combinatorial library, as described below, of potential antagonists, and screening the library members for binding to the protein or other molecule in question. The binding members may then be further screened for the ability to antagonize the biological activity of the target. The antagonists may be used therapeutically, or, in suitably labeled or immobilized form, diagnostically.

If the cDNA is related to a known protein, then substances known to interact with that protein (e.g., agonists, antagonists, substrates, receptors, second messengers, regulators, and so forth), and binding molecules which bind them, are also of utility. Such binding molecules can likewise be identified by screening a combinatorial library.

Isolation of Full Length cDNAs Using Partial cDNAs as Probes

If it is determined that a cDNA of the present invention is a partial cDNA, and the cognate full length cDNA is not listed in a sequence database, the available cDNA may be used as a hybridization probe to isolate the full-length cDNA from a suitable cDNA library.

Stringent hybridization conditions are appropriate, that is, conditions in which the hybridization temperature is 5-10 deg. C. below the Tm of the cDNA as a perfect duplex.

Identification and Isolation of Homologous Genes/cDNAs Using a cDNA Probe

It may be that the sequence databases available do not include the sequence of any homologous gene, or at least of the homologous gene for a species of interest. However, given the cDNAs set forth above, one may readily obtain the homologous gene.

The possession of one cDNA (the “starting DNA”) greatly facilitates the isolation of homologous genes/cDNAs. If the clone in question only features a partial cDNA, this partial cDNA may first be used as a probe to isolate the corresponding full length cDNA for the same species, and that the latter may be used as the starting DNA in the search for homologous genes.

The starting DNA, or a fragment thereof, is used as a hybridization probe to screen a cDNA or genomic DNA library for clones containing inserts which encode either the entire homologous protein, or a recognizable fragment thereof. The minimum length of the hybridization probe is dictated by the need for specificity. If the size of the library in bases is L, and the GC content is 50%, then the probe should have a length of at least 1, where L=41. This will yield, on average, a single perfect match in random DNA of L bases. The human cDNA library is about 108 bases and the human genomic DNA library is about 1010 bases.

The library is preferably derived from an organism which is known, on biochemical evidence, to produce a homologous protein, and more preferably from the genomic DNA or mRNA of cells of that organism which are likely to be relatively high producers of that protein. A cDNA library (which is derived from an mRNA library) is especially preferred.

If the organism in question is known to have substantially different codon preferences from that of the organism whose relevant cDNA or genomic DNA is known, a synthetic hybridization probe may be used which encodes the same amino acid sequence but whose codon utilization is more similar to that of the DNA of the target organism. Alternatively, the synthetic probe may employ inosine as a substitute for those bases which are most likely to be divergent, or the probe may be a mixed probe which mixes the codons for the source DNA with the preferred codons (encoding the same amino acid) for the target organism.

By routine methods, the Tm of a perfect duplex of starting DNA is determined. One may then select a hybridization temperature which is sufficiently lower than the perfect duplex Tm to allow hybridization of the starting DNA (or other probe) to a target DNA which is divergent from the starting DNA. A 1% sequence divergence typically lowers the Tm of a duplex by 1-2° C., and the DNAs encoding homologous proteins of different species typically have sequence identities of around 50-80%. Preferably, the library is screened under conditions where the temperature is at least 20° C., more preferably at least 50° C., below the perfect duplex Tm. Since salt reduces the Tm, one ordinarily would carry out the search for DNAs encoding highly homologous proteins under relatively low salt hybridization conditions, e.g., <1M NaCl. The higher the salt concentration, and/or the lower the temperature, the greater the sequence divergence which is tolerated.

For the use of probes to identify homologous genes in other species, see, e.g., Schwinn, et al., J. Biol. Chem., 265:8183-89 (1990) (hamster 67-bp cDNA probe vs. human leukocyte genomic library; human 0.32 kb DNA probe vs. bovine brain cDNA library, both with hybridization at 42° C. in 6×SSC); Jenkins et al., J. Biol. Chem., 265:19624-31 (1990) (Chicken 770-bp cDNA probe vs. human genomic libraries; hybridization at 40° C. in 50% formamide and 5×SSC); Murata et al., J. Exp. Med., 175:341-51 (1992) (1.2-kb mouse cDNA probe v. human eosinophl cDNA library; hybridization at 65° C. in 6×SSC); Guyer et al., J. Biol. Chem., 265:17307-17 (1990) (2.95-kb human genomic DNA probe vs. porcine genomic DNA library; hybridization at 42° C. in 5×SSC). The conditions set forth in these articles may each be considered suitable for the purpose of isolating homologous genes.

Homologous Proteins and DNAs

A human protein can be said to be identifiable as homologous to a mouse cDNA clone if

(1) its sequence can be aligned to the mouse cDNA clone, using BlastX with the default parameters set forth below, and the expected value (E) of the alignment (the probability that such an alignment would have occurred by chance alone) is less than e-10, or

(2) it is encoded by a human gene whose cDNA can be aligned to the mouse cDNA clone, using BlastN with the default parameters set forth below, and the E value of the alignment is less than e-10.

Desirably, both conditions are satisfied.

Preferably, for either or both conditions, the E value is less than e-15, more preferably less than e-20, still more preferably less than e-40, even more preferably less than e-60, considerably more preferably less than e-80, and most preferably less than e-100.

BlastN and BlastX report very low expected values as “0.0”. This does not truly mean that the expected value is exactly zero (since any alignment could occur by chance), but merely that it is so infinitesimal that it is not reported. The documentation does not state the cutoff value, but the data below includes alignments with explicit E values as low as e-178 (624 bits), while a score of 636 bits was reported as “0.0”.

Functionally homologous human proteins are also of interest. A human protein may be said to be functionally homologous to the mouse cDNA clone if (1) there is a mouse protein which is encoded by a mouse gene whose cDNA can be aligned to the mouse cDNA clone, using BlastX with the default parameters set forth below, and the E value of the alignment is less than e-50, and (2) the human protein has at least one biological activity in common with the mouse protein.

The human proteins of interest also include those that are substantially and/or conservatively identical (as defined below) to the homologous and/or functionally homologous human proteins defined above.

Relevance of Favorable and Unfavorable Genes

If a gene is down-regulated in protected mammals, or up-regulated in damaged mammals, (i.e., an “unfavorable gene”) then several utilities are apparent.

First, the complementary strand of the gene, or a portion thereof, may be used in labeled form as a hybridization probe to detect messenger RNA and thereby monitor the level of expression of the gene in a subject. Elevated levels are indicative of damage, or possibly of a propensity to damage, and clinicians may take appropriate preventative, curative or ameliorative action.

Secondly, the messenger RNA product (or equivalent cDNA), the protein product, or a binding molecule specific for that product (e.g., an antibody which binds the product), or a downstream product which mediates the activity (e.g., a signaling intermediate) or a binding molecule (e.g., an antibody) therefor, may be used, preferably in labeled or immobilized form, as an assay reagent in an assay for said nucleic acid product, protein product, or downstream product (e.g., a signaling intermediate). Again, elevated levels are indicative of a present or future problem.

Thirdly, an agent which down-regulates expression of the gene may be used to reduce levels of the corresponding protein and thereby inhibit further damage to the kidney. This agent could inhibit transcription of the gene in the subject, or translation of the corresponding messenger RNA. Possible inhibitors of transcription and translation include antisense molecules and repressor molecules. The agent could also inhibit a post-translational modification (e.g., glycosylation, phosphorylation, cleavage, GPI attachment) required for activity, or post-translationally modify the protein so as to inactivate it. Or it could be an agent which down- or up-regulated a positive or negative regulatory gene, respectively.

Fourthly, an agent which is an antagonist of the messenger RNA product or protein product of the gene, or of a downstream product through which its activity is manifested (e.g., a signaling intermediate), may be used to inhibit its activity. This antagonist could be an antibody.

Fifthly, an agent which degrades, or abets the degradation of, that messenger RNA, its protein product or a downstream product which mediates its activity (e.g., a signaling intermediate), may be used to curb the effective period of activity of the protein.

If a gene is up-regulated in protected mammals, or down-regulated in damaged animals then the utilities are converse to those stated above.

First, the complementary strand of the gene, or a portion thereof, may be used in labeled form as a hybridization probe to detect messenger RNA and thereby monitor the level of expression of the gene in a subject. Depressed levels are indicative of damage, or possibly of a propensity to damage, and clinicians may take appropriate preventative, curative or ameliorative action.

Secondly, the messenger RNA product, the equivalent cDNA, protein product, or a binding molecule specific for those products, or a downstream product, or a signaling intermediate, or a binding molecule therefor, may be used, preferably in labeled or immobilized form, as an assay reagent in an assay for said protein product or downstream product. Again, depressed levels are indicative of a present or future problem.

Thirdly, an agent which up-regulates expression of the gene may be used to increase levels of the corresponding protein and thereby inhibit further damage to the kidney. By way of example, it could be a vector which carries a copy of the gene, but which expresses the gene at higher levels than does the endogenous expression system. Or it could be an agent which up- or down-regulates a positive or negative regulatory gene.

Fourthly, an agent which is an agonist of the protein product of the gene, or of a downstream product through which its activity (of inhibition of kidney damage) is manifested, or of a signaling intermediate may be used to foster its activity.

Fifthly, an agent which inhibits the degradation of that protein product or of a downstream product or of a signaling intermediate may be used to increase the effective period of activity of the protein.

Relevance of Antibody-Related Genes

Some of the disclosed clones (e.g., A8, A34, A39, A48, E39, G38) appear to be related to antibody genes. It is widely accepted that diabetes is a microvasculature problem. Elevated levels of glucose or lack of insulin or insulin resistance result in pathological problems with capillaries. The lack of proper blood supply to a variety of tissues results in tissue damage including neuronal problems (neurology), kidney problems (nephropathy), cardiovascular problems, and eye problems (proliferative diabetic retinopathy). Severe blood vessel problems (macrovascular disorders) can lead to necrotic tissue that often results in amputations.

Also, there is considerable evidence that the underlying etiology of Type I diabetes is a problem with the immune system, in that an autoimmune reaction is mounted that results in destruction of the Beta (insulin producing) cells of the pancreas.

Immune reactions have been shown to be involved in glomerulosclerosis, see Chen, et al., Nephron, 78: 63-72 (1998) and Linder, et al., Clin. Immunol. Immunopathol., 1:104-21 (1972), and to occur in transgenic mice expressing bovine GH and exhibiting arthritis, see Ogueta, supra.

Thus, it is entirely possible that in a type II diabetic (and in other tissues of Type I diabetics), autoimmune reactions take place. The scenario could be as follows: due to inappropriate blood flow to a given tissue (for example, the kidney), pathological changes occur. These changes could be in the form of glomerulsclerosis, necrosis, or a variety of other problems. Proteins found in the tissue may become altered during these pathological changes (for example inappropriate glycosylation, phosphorylation, protein cleavage) and this ultimately results in the exposure of “new” or foreign epitopes to the host. Thus, an autoimmune reaction could be mounted against these proteins that impact the destruction of the tissue.

It follows that inhibition of these autoimmune antibodies can be beneficial.

Conceivably, as titers of these autoimmune antibodies rise, a new immune response, featuring anti-idiotypic antibodies, will occur; such antibodies would be “favorable”.

Genes/Proteins of Interest

Growth hormone (GH) is thought to play a role in the progression of kidney damage in mice. Bovine (b) GH transgenic mice possess elevated levels of insulin-like growth factor-1 (IGF-1), elevated levels of insulin, a giant phenotype, and develop severe kidney damage resulting in death within 7-12 months. GH antagonist mice have decreased levels of IGF-1, a dwarf phenotype, and fail to develop kidney damage, even when made diabetic by treatment with streptozotocin (Stz). Therefore, the bGH mice that develop kidney damage independent of diabetes but with characteristics similar to Stz-induced diabetic nephropathy may provide a model system for determining GH's role in the progression of kidney damage. As a consequence of determining the genes activated or inactivated in correlation with the morphology of the kidney as it progresses towards glomerulosclerosis, agents may be identified which can protect kidneys from glomerulosclerosis and other kidney damage.

Favorable genes/proteins include the following:
SON protein
vacuolar adenosine triphosphatase
Ac39/physophilin
ubiquitin protein ligase Nedd-4
kindney androgen regulated protein
claudins
heat shock proteins
far upstream element (FUSE) binding protein

Unfavorable genes/proteins include the following:

3 beta-hydroxysteroid dehydrogenase, especially of type IV or V.
disabled-2 (dab-2) p96
palmitylated serine/threonine kinase
tumor differentially expressed (TDE) 1
cytochrome oxidase III
ERG2 protein
TLH29 protein precursor
glutathoione peroxidase 3
   Mutant Proteins

The present invention also contemplates mutant proteins (peptides) which are substantially identical (as defined below) to the parental protein (peptide). In general, the fewer the mutations, the more likely the mutant protein is to retain the activity of the parental protein. The effect of mutations is usually (but not always) additive. Certain individual mutations are more likely to be tolerated than others.

A protein is more likely to tolerate a mutation which
(a) is a substitution rather than an insertion or deletion;
(b) is an insertion or deletion at the terminus, rather than internally, or, if internal, is at a domain boundary, or a loop or turn, rather than in an alpha helix or beta strand;
(c) affects a surface residue rather than an interior residue;
(d) affects a part of the molecule distal to the binding site;
(e) is a substitution of one amino acid for another of similar size, charge, and/or hydrophobicity, and does not destroy a disulfide bond or other crosslink; and
(f) is at a site which is subject to substantial variation among a family of homologous proteins to which the protein of interest belongs.
   These considerations can be used to design functional mutants.
   Surface vs. Interior Residues

Charged residues almost always lie on the surface of the protein. For uncharged residues, there is less certainty, but in general, hydrophilic residues are partitioned to the surface and hydrophobic residues to the interior. Of course, for a membrane protein, the membrane-spanning segments are likely to be rich in hydrophobic residues.

Surface residues may be identified experimentally by various labeling techniques, or by 3-D structure mapping techniques like X-ray diffraction and NMR. A 3-D model of a homologous protein can be helpful.

Binding Site Residues

Residues forming the binding site may be identified by (1) comparing the effects of labeling the surface residues before and after completing the protein to its target, (2) labeling the binding site directly with affinity ligands, (3) fragmenting the protein and testing the fragments for binding activity, and (4) systematic mutagenesis (e.g., alanine-scanning mutagenesis) to determine which mutants destroy binding. If the binding site of a homologous protein is known, the binding site may be postulated by analogy.

Protein libraries may be constructed and screened that a large family (e.g., 10⁸) of related mutants may be evaluated simultaneously.

Hence, the mutations are preferably, conservative modifications as defined below.

“Substantially Identical”

A mutant protein (peptide) is substantially identical to a reference protein (peptide) if (a) it has at least 10% of a specific binding activity or a non-nutritional biological activity of the reference protein, and (b) is at least 50% identical in amino acid sequence to the reference protein (peptide). It is “substantially structurally identical” if condition (b) applies, regardless of (a).

Percentage amino acid identity is determined by aligning the mutant and reference sequences according to a rigorous dynamic programming algorithm which globally aligns their sequences to maximize their similarity, the similarity being scored as the sum of scores for each aligned pair according to an unbiased PAM250 matrix, and a penalty for each internal gap of −12 for the first null of the gap and −4 for each additional null of the same gap. The percentage identity is the number of matches expressed as a percentage of the adjusted (i.e., counting inserted nulls) length of the reference sequence.

A mutant DNA sequence is substantially identical to a reference DNA sequence if they are structural sequences, and encoding mutant and reference proteins which are substantially identical as described above.

If instead they are regulatory sequences, they are substantially identical if the mutant sequence has at least 10% of the regulatory activity of the reference sequence, and is at least 50% identical in nucleotide sequence to the reference sequence. Percentage identity is determined as for proteins except that matches are scored +5, mismatches −4, the gap open penalty is −12, and the gap extension penalty (per additional null) is −4.

Preferably, sequence which are substantially identical exceed the minimum identity of 50% e.g., are 51%, 66%, 75%, 80%, 85%, 90%, 95% or 99% identical in sequence.

DNA sequences may also be considered “substantially identical” if they hybridize to each other under stringent conditions, i.e., conditions at which the Tm of the heteroduplex of the one strand of the mutant DNA and the more complementary strand of the reference DNA is not in excess of 10° C. less than the Tm of the reference DNA homoduplex. Typically this will correspond to a percentage identity of 85-90%.

“Conservative Modifications”

“Conservative modifications” are defined as
(a) conservative substitutions of amino acids as hereafter defined; or
(b) single or multiple insertions (extension) or deletions (truncation) of amino acids at the termini.

Conservative modifications are preferred to other modifications. Conservative substitutions are preferred to other conservative modifications.

“Semi-Conservative Modifications” are modifications which are not conservative, but which are (a) semi-conservative substitutions as hereafter defined; or (b) single or multiple insertions or deletions internally, but at interdomain boundaries, in loops or in other segments of relatively high mobility. Semi-conservative modifications are preferred to nonconservative modifications. Semi-conservative substitutions are preferred to other semi-conservative modifications.

Non-conservative substitutions are preferred to other non-conservative modifications.

The term “conservative” is used here in an a priori sense, i.e., modifications which would be expected to preserve 3D structure and activity, based on analysis of the naturally occurring families of homologous proteins and of past experience with the effects of deliberate mutagenesis, rather than post facto, a modification already known to conserve activity. Of course, a modification which is conservative a priori may, and usually is, also conservative post facto.

Preferably, except at the termini, no more than about five amino acids are inserted or deleted at a particular locus, and the modifications are outside regions known to contain binding sites important to activity.

Preferably, insertions or deletions are limited to the termini.

A conservative substitution is a substitution of one amino acid for another of the same exchange group, the exchange groups being defined as follows
I Gly, Pro, Ser, Ala (Cys) (and any nonbiogenic, neutral amino acid with a hydrophobicity not exceeding that of the aforementioned a.a.'s)
II Arg, Lys, His (and any nonbiogenic, positively-charged amino acids)
III Asp, Glu, Asn, Gln (and any nonbiogenic negatively-charged amino acids)
IV Leu, Ile, Met, Val (Cys) (and any nonbiogenic, aliphatic, neutral amino acid with a hydrophobicity too high for I above)
V Phe, Trp, Tyr (and any nonbiogenic, aromatic neutral amino acid with a hydrophobicity too high for I above).

Note that Cys belongs to both I and IV.

Residues Pro, Gly and Cys have special conformational roles. Cys participates in formation of disulfide bonds. Gly imparts flexibility to the chain. Pro imparts rigidity to the chain and disrupts a helices. These residues may be essential in certain regions of the polypeptide, but substitutable elsewhere.

One, two or three conservative substitutions are more likely to be tolerated than a larger number.

“Semi-conservative substitutions” are defined herein as being substitutions within supergroup I/II/III or within supergroup IV/V, but not within a single one of groups I-V. They also include replacement of any other amino acid with alanine. If a substitution is not conservative, it preferably is semi-conservative.

“Non-conservative substitutions” are substitutions which are not “conservative” or “semi-conservative”.

“Highly conservative substitutions” are a subset of conservative substitutions, and are exchanges of amino acids within the groups Phe/Tyr/Trp, Met/Leu/Ile/Val, His/Arg/Lys, Asp/Glu and Ser/Thr/Ala. They are more likely to be tolerated than other conservative substitutions. Again, the smaller the number of substitutions, the more likely they are to be tolerated.

“Conservatively Identical”

A protein (peptide) is conservatively identical to a reference protein (peptide) it differs from the latter, if at all, solely by conservative modifications, the protein (peptide remaining at least seven amino acids long if the reference protein (peptide) was at least seven amino acids long.

A protein is at least semi-conservatively identical to a reference protein (peptide) if it differs from the latter, if at all, solely by semi-conservative or conservative modifications.

A protein (peptide) is nearly conservatively identical to a reference protein (peptide) if it differs from the latter, if at all, solely by one or more conservative modifications and/or a single nonconservative substitution.

It is highly conservatively identical if it differs, if at all, solely by highly conservative substitutions. Highly conservatively identical proteins are preferred to those merely conservatively identical. An absolutely identical protein is even more preferred.

The core sequence of a reference protein (peptide) is the largest single fragment which retains at least 10% of a particular specific binding activity, if one is specified, or otherwise of at least one specific binding activity of the referent. If the referent has more than one specific binding activity, it may have more than one core sequence, and these may overlap or not.

If it is taught that a peptide of the present invention may have a particular similarity relationship (e.g., markedly identical) to a reference protein (peptide), preferred peptides are those which comprise a sequence having that relationship to a core sequence of the reference protein (peptide), but with internal insertions or deletions in either sequence excluded. Even more preferred peptides are those whose entire sequence has that relationship, with the same exclusion, to a core sequence of that reference protein (peptide).

Library

The term “library” generally refers to a collection of chemical or biological entities which are related in origin, structure and/or function, and which can be screened simultaneously for a property of interest.

Libraries may be classified by how they are constructed (natural vs. artificial diversity; combinatorial vs. noncombinatorial), how they are screened (hybridization, expression, display), or by the nature of the screened library members (peptides, nucleic acids, etc.).

In a “natural diversity” library, essentially all of the diversity arose without human intervention. This would be true, for example, of messenger RNA extracted from a non-engineered cell.

In a “synthetic diversity” library, essentially all of the diversity arose deliberately as a result of human intervention. This would be true for example of a combinatorial library; note that a small level of natural diversity could still arise as a result of spontaneous mutation. It would also be true of a noncombinatorial library of compounds collected from diverse sources, even if they were all natural products.

In a “non-natural diversity” library, at least some of the diversity arose deliberately through human intervention.

In a “controlled origin” library, the source of the diversity is limited in some way. A limitation might be to cells of a particular individual, to a particular species, or to a particular genus, or, more complexly, to individuals of a particular species who are of a particular age, sex, physical condition, geographical location, occupation and/or familial relationship. Alternatively or additionally, it might be to cells of a particular tissue or organ. Or it could be cells exposed to particular pharmacological, environmental, or pathogenic conditions. Or the library could be of chemicals, or a particular class of chemicals, produced by such cells.

In a “controlled structure” library, the library members are deliberately limited by the production conditions to particular chemical structures. For example, if they are oligomers, they may be limited in length and monomer composition, e.g. hexapeptides composed of the twenty genetically encoded amino acids.

Hybridization Library

In a hybridization library, the library members are nucleic acids, and are screened using a nucleic acid hybridization probe. Bound nucleic acids may then be sequenced.

Expression Library

In an expression library, the screened library members are gene expression products, but one may also speak of an underlying library of genes encoding those products. The library is made by subcloning DNA encoding the library members (or portions thereof) into expression vectors (or into cloning vectors which subsequently are used to construct expression vectors), each vector comprising an expressible gene encoding a particular library member, introducing the expression vectors into suitable cells, and expressing the genes so the expression products are produced.

In one embodiment, the expression products are secreted, so the library can be screened using an affinity reagent, such as an antibody or receptor. The bound expression products may be sequenced directly, or their sequences inferred by, e.g., sequencing at least the variable portion of the encoding DNA.

In a second embodiment, the cells are lysed, thereby exposing the expression products, and the latter are screened with the affinity reagent.

In a third embodiment, the cells express the library members in such a manner that they are displayed on the surface of the cells, or on the surface of viral particles produced by the cells. (See display libraries, below).

In a fourth embodiment, the screening is not for the ability of the expression product to bind to an affinity reagent, but rather for its ability to alter the phenotype of the host cell in a particular detectable manner. Here, the screened library members are transformed cells, but there is a first underlying library of expression products which mediate the behavior of the cells, and a second underlying library of genes which encode those products.

Display Library

In a display library, the library members are each conjugated to, and displayed upon, a support of some kind. The support may be living (a cell or virus), or nonliving (e.g., a bead or plate).

If the support is a cell or virus, display will normally be effectuated by expressing a fusion protein which comprises the library member, a carrier moiety allowing integration of the fusion protein into the surface of the cell or virus, and optionally a lining moiety. In a variation on this theme, the cell coexpresses a first fusion comprising the library member and a linking moiety L1, and a second fusion comprising a linking moiety L2 and the carrier moiety. L1 and L2 interact to associate the first fusion with the second fusion and hence, indirectly, the library member with the surface of the cell or virus.

Soluble Library

In a soluble library, the library members are free in solution. A soluble library may be produced directly, or one may first make a display library and then release the library members from their supports.

Encapsulated Library

In an encapsulated library, the library members are inside cells or liposomes. Generally speaking, encapsulated libraries are used to store the library members for future use; the members are extracted in some way for screening purposes. However, if they differentially affect the phenotype of the cells, they may be screened indirectly by screening the cells.

cDNA Library

A cDNA library is usually prepared by extracting RNA from cells of particular origin, fractionating the RNA to isolate the messenger RNA (mRNA has a poly(A) tail, so this is usually done by oligo-dT affinity chromatography), synthesizing complementary DNA (cDNA) using reverse transcriptase, DNA polymerase, and other enzymes, subcloning the cDNA into vectors, and introducing the vectors into cells. Often, only mRNAs or cDNAs of particular sizes will be used, to make it more likely that the cDNA encodes a functional polypeptide.

A cDNA library explores the natural diversity of the transcribed DNAs of cells from a particular source. It is not a combinatorial library.

A cDNA library may be used to make a hybridization library, or it may be used as an (or to make) expression library.

Genomic DNA Library

A genomic DNA library is made by extracting DNA from a particular source, fragmenting the DNA, isolating fragments of a particular size range, subcloning the DNA fragments into vectors, and introducing the vectors into cells.

Like a cDNA library, a genomic DNA library is a natural diversity library, and not a combinatorial library. A genomic DNA library may be used the same way as a cDNA library.

Synthetic DNA Library

A synthetic DNA library may be screened directly (as a hybridization library), or used in the creation of an expression or display library of peptides/proteins.

Combinatorial Libraries

The term “combinatorial library” refers to a library in which the individual members are either systematic or random combinations of a limited set of basic elements, the properties of each member being dependent on the choice and location of the elements incorporated into it. Typically, the members of the library are at least capable of being screened simultaneously. Randomization may be complete or partial; some positions may be randomized and others predetermined, and at random positions, the choices may be limited in a predetermined manner. The members of a combinatorial library may be oligomers or polymers of some kind, in which the variation occurs through the choice of monomeric building block at one or more positions of the oligomer or polymer, and possibly in terms of the connecting linkage, or the length of the oligomer or polymer, too. Or the members may be nonoligomeric molecules with a standard core structure, like the 1,4-benzodiazepine structure, with the variation being introduced by the choice of substituents at particular variable sites on the core structure. Or the members may be nonoligomeric molecules assembled like a jigsaw puzzle, but wherein each piece has both one or more variable, moieties (contributing to library diversity) and one or more constant moieties (providing the functionalities for coupling the piece in question to other pieces).

Thus, in a typical combinatorial library, chemical building blocks are at least partially randomly combined into a large number (as high as 10E15) of different compounds, which are then simultaneously screened for binding (or other) activity against one or more targets.

In a “simple combinatorial library”, all of the members belong to the same class of compounds (e.g., peptides) and can be synthesized simultaneously. A “composite combinatorial library” is a mixture of two or more simple libraries, e.g., DNAs and peptides, or peptides, peptoids, and PNAs, or benzodiazepines and carbamates. The number of component simple libraries in a composite library will, of course, normally be smaller than the average number of members in each simple library, as otherwise the advantage of a library over individual synthesis is small.

Libraries of thousands, even millions, of random oligopeptides have been prepared by chemical synthesis (Houghten et al., Nature, 354:84-6(1991)), or gene expression (Marks et al., J Mol Biol, 222:581-97(1991)), displayed on chromatographic supports (Lam et al., Nature, 354:82-4(1991)), inside bacterial cells (Colas et al., Nature, 380:548-550(1996)), on bacterial pili (Lu Bio/Technology, 13:366-372(1990)), or phage (Smith, Science, 228:1315-7(1985)), and screened for binding to a variety of targets including antibodies (Valadon et al., J Mol Biol, 261:11-22(1996)), cellular proteins (Schmitz et al., J Mol Biol, 260:664-677(1996)), viral proteins (Hong and Boulanger, Embo J, 14:4714-4727(1995)), bacterial proteins (Jacobsson and Frykberg, Biotechniques, 18:878-885(1995)), nucleic acids (Cheng et al., Gene, 171:1-8(1996)), and plastic (Siani et al., J Chem Inf Comput Sci, 34:588-593(1994)).

Libraries of proteins (Ladner, U.S. Pat. No. 4,664,989), peptoids (Simon et al., Proc Natl Acad Sci USA, 89:9367-71(1992)), nucleic acids (Ellington and Szostak, Nature, 246:818(1990)), carbohydrates, and small organic molecules (Eichler et al., Med Res Rev, 15:481-96(1995)) have also been prepared or suggested for drug screening purposes.

The first combinatorial libraries were composed of peptides or proteins, in which all or selected amino acid positions were randomized. Peptides and proteins can exhibit high and specific binding activity, and can act as catalysts. In consequence, they are of great importance in biological systems.

Nucleic acids have also been used in combinatorial libraries. Their great advantage is the ease with which a nucleic acid with appropriate binding activity can be amplified. As a result, combinatorial libraries composed of nucleic acids can be of low redundancy and hence, of high diversity.

There has also been much interest in combinatorial libraries based on small molecules, which are more suited to pharmaceutical use, especially those which, like benzodiazepines, belong to a chemical class which has already yielded useful pharmacological agents. The techniques of combinatorial chemistry have been recognized as the most efficient means for finding small molecules that act on these targets. At present, small molecule combinatorial chemistry involves the synthesis of either pooled or discrete molecules that present varying arrays of functionality on a common scaffold. These compounds are grouped in libraries that are then screened against the target of interest either for binding or for inhibition of biological activity.

The size of a library is the number of molecules in it. The simple diversity of a library is the number of unique structures in it. There is no formal minimum or maximum diversity. If the library has a very low diversity, the library has little advantage over just synthesizing and screening the members individually. If the library is of very high diversity, it may be inconvenient to handle, at least without automatizing the process. The simple diversity of a library is preferably at least 10, 10E2, 10E3, 10E4, 10E6, 10E7, 10E8 or 10E9, the higher the better under most circumstances. The simple diversity is usually not more than 10E15, and more usually not more than 10E10.

The average sampling level is the size divided by the simple diversity. The expected average sampling level must be high enough to provide a reasonable assurance that, if a given structure were expected, as a consequence of the library design, to be present, that the actual average sampling level will be high enough so that the structure, if satisfying the screening criteria, will yield a positive result when the library is screened. Thus, the preferred average sampling level is a function of the detection limit, which in turn is a function of the strength of the signal to be screened.

There are more complex measures of diversity than simple diversity. These attempt to take into account the degree of structural difference between the various unique sequences. These more complex measures are usually used in the context of small organic compound libraries, see below.

The library members may be presented as solutes in solution, or immobilized on some form of support. In the latter case, the support may be living (cell, virus) or nonliving (bead, plate, etc.). The supports may be separable (cells, virus particles, beads) so that binding and nonbinding members can be separated, or nonseparable (plate). In the latter case, the members will normally be placed on addressable positions on the support. The advantage of a soluble library is that there is no carrier moiety that could interfere with the binding of the members to the support. The advantage of an immobilized library is that it is easier to identify the structure of the members which were positive.

When screening a soluble library, or one with a separable support, the target is usually immobilized. When screening a library on a nonseparable support, the target will usually be labeled.

Oligonucleotide Libraries

An oligonucleotide library is a combinatorial library, at least some of whose members are single-stranded oligonucleotides having three or more nucleotides connected by phosphodiester or analogous bonds. The oligonucleotides may be linear, cyclic or branched, and may include non-nucleic acid moieties. The nucleotides are not limited to the nucleotides normally found in DNA or RNA. For examples of nucleotides modified to increase nuclease resistance and chemical stability of aptamers, see Chart 1 in Osborne and Ellington, Chem. Rev., 97: 349-70 (1997). For screening of RNA, see Ellington and Szostak, Nature, 346: 818-22 (1990). There is no formal minimum or maximum size for these oligonucleotides. However, the number of conformations which an oligonucleotide can assume increases exponentially with its length in bases. Hence, a longer oligonucleotide is more likely to be able to fold to adapt itself to a protein surface. On the other hand, while very long molecules can be synthesized and screened, unless they provide a much superior affinity to that of shorter molecules, they are not likely to be found in the selected population, for the reasons explained by Osborne and Ellington (1997). Hence, the libraries of the present invention are preferably composed of oligonucleotides having a length of 3 to 100 bases, more preferably 15 to 35 bases. The oligonucleotides in a given library may be of the same or of different lengths.

Oligonucleotide libraries have the advantage that libraries of very high diversity (e.g., 1015) are feasible, and binding molecules are readily amplified in vitro by polymerase chain reaction (PCR). Moreover, nucleic acid molecules can have very high specificity and affinity to targets.

In a preferred embodiment, this invention prepares and screens oligonucleotide libraries by the SELEX method, as described in King and Famulok, Molec. Biol. Repts., 20: 97-107 (1994); L. Gold, C. Tuerk. Methods of producing nucleic acid ligands, U.S. Pat. No. 5,595,877; Oliphant et al. Gene 44:177 (1986).

The term “aptamer” is conferred on those oligonucleotides which bind the target protein. Such aptamers may be used to characterize the target protein, both directly (through identification of the aptamer and the points of contact between the aptamer and the protein) and indirectly (by use of the aptamer as a ligand to modify the chemical reactivity of the protein).

In a classic oligonuclotide, each nucleotide (monomeric unit) is composed of a phosphate group, a sugar moiety, and either a purine or a pyrimidine base. In DNA, the sugar is deoxyribose and in RNA it is ribose. The nucleotides are linked by 5′-3′ phosphodiester bonds.

The deoxyribose phosphate backbone of DNA can be modified to increase resistance to nuclease and to increase penetration of cell membranes. Derivatives such as mono- or dithiophosphates, methyl phosphonates, boranophosphates, formacetals, carbamates, siloxanes, and dimethylenethio-sulfoxideo- and -sulfono-linked species are known in the art.

Peptide Library

A peptide is composed of a plurality of amino acid residues joined together by peptidyl (—NHCO—) bonds. A biogenic peptide is a peptide in which the residues are all genetically encoded amino acid residues; it is not necessary that the biogenic peptide actually be produced by gene expression.

Amino acids are the basic building blocks with which peptides and proteins are constructed. Amino acids possess both an amino group (—NH₂) and a carboxylic acid group COOH). Many amino acids, but not all, have the alpha amino acid structure NH₂—CHR—COOH, where R is hydrogen, or any of a variety of functional groups.

Twenty amino acids are genetically encoded: Alanine, Arginine, Asparagine, Aspartic Acid, Cysteine, Glutamic Acid, Glutamine, Glycine, Histidine, Isoleucine, Leucine, Lysine, Methionine, Phenylalanine, Proline, Serine, Threonine, Tryptophan, Tyrosine, and Valine. Of these, all save Glycine are optically isomeric, however, only the L-form is found in humans. Nevertheless, the D-forms of these amino acids do have biological significance; D-Phe, for example, is a known analgesic.

Many other amino acids are also known, including: 2-Aminoadipic acid; 3-Aminoadipic acid; beta-Aminopropionic acid; 2-Aminobutyric acid; 4-Aminobutyric acid (Piperidinic acid); 6-Aminocaproic acid; 2-Aminoheptanoic acid; 2-Aminoisobutyric acid, 3-Aminoisobutyric acid; 2-Aminopimelic acid; 2,4-Diaminobutyric acid; Desmosine; 2,2′-Diaminopimelic acid; 2,3-Diaminopropionic acid; N-Ethylglycine; N-Ethylasparagine, Hydroxylysine; allo-Hydroxylysine; 3-Hydroxyproline; 4-Hydroxyproline; Isodesmosine; allo-Isoleucine; N-Methylglycine (Sarcosine); N-Methylisoleucine; N-Methylvaline; Norvaline; Norleucine; and Ornithine.

Peptides are constructed by condensation of amino acids and/or smaller peptides. The amino group of one amino acid (or peptide) reacts with the carboxylic acid group of a second amino acid (or peptide) to form a peptide (—NHCO—) bond, releasing one molecule of water. Therefore, when an amino acid is incorporated into a peptide, it should, technically speaking, be referred to as an amino acid residue. The core of that residue is the moiety which excludes the —NH and —CO linking functionalities which connect it to other residues. This moiety consists of one or more main chain atoms (see below) and the attached side chains.

The main chain moiety of each amino acid consists of the —NH and —CO linking functionalities and a core main chain moiety. Usually the latter is a single carbon atom. However, the core main chain moiety may include additional carbon atoms, and may also include nitrogen, oxygen or sulfur atoms, which together form a single chain. In a preferred embodiment, the core main chain atoms consist solely of carbon atoms.

The side chains are attached to the core main chain atoms. For alpha amino acids, in which the side chain is attached to the alpha carbon, the C-1, C-2 and N-2 of each residue form the repeating unit of the main chain, and the word “side chain” refers to the C-3 and higher numbered carbon atoms and their substituents. It also includes H atoms attached to the main chain atoms.

Amino acids may be classified according to the number of carbon atoms which appear in the main chain between the carbonyl carbon and amino nitrogen atoms which participate in the peptide bonds. Among the 150 or so amino acids which occur in nature, alpha, beta, gamma and delta amino acids are known. These have 1-4 intermediary carbons. Only alpha amino acids occur in proteins. Proline is a special case of an alpha amino acid; its side chain also binds to the peptide bond nitrogen.

For beta and higher order amino acids, there is a choice as to which main chain core carbon a side chain other than H is attached to. The preferred attachment site is the C-2 (alpha) carbon, i.e., the one adjacent to the carboxyl carbon of the —CO linking functionality. It is also possible for more than one main chain atom to carry a side chain other than H. However, in a preferrred embodiment, only one main chain core atom carries a side chain other than H.

A main chain carbon atom may carry either one or two side chains; one is more common. A side chain may be attached to a main chain carbon atom by a single or a double bond; the former is more common.

A simple combinatorial peptide library is one whose members are peptides having three or more amino acids connected via peptide bonds.

The peptides may be linear, branched, or cyclic, and may covalently or noncovalently include nonpeptidyl moieties. The amino acids are not limited to the naturally occurring or to the genetically encoded amino acids.

A biased peptide library is one in which one or more (but not all) residues of the peptides are constant residues.

Cyclic Peptides

Many naturally occurring peptides are cyclic. Cyclization is a common mechanism for stabilization of peptide conformation thereby achieving improved association of the peptide with its ligand and hence improved biological activity. Cyclization is usually achieved by intra-chain cystine formation, by formation of peptide bond between side chains or between N- and C-terminals. Cyclization was usually achieved by peptides in solution, but several publications have appeared that describe cyclization of peptides on beads.

A peptide library may be an oligopeptide library or a protein library.

Oligopeptides

Preferably, the oligopeptides are at least five, six, seven or eight amino acids in length. Preferably, they are composed of less than 50, more preferably less than 20 amino acids.

In the case of an oligopeptide library, all or just some of the residues may be variable. The oligopeptide may be unconstrained, or constrained to a particular conformation by, e.g., the participation of constant cysteine residues in the formation of a constraining disulfide bond.

Proteins

Proteins, like oligopeptides, are composed of a plurality of amino acids, but the term protein is usually reserved for longer peptides, which are able to fold into a stable conformation. A protein may be composed of two or more polypeptide chains, held together by covalent or noncovalent crosslinks.

A peptide is considered a protein if it (1) is at least 50 amino acids long, or (2) has at least two stabilizing covalent crosslinks (e.g., disulfide bonds). Thus, conotoxins are considered proteins.

Usually, the proteins of a protein library will be characterizable as having both constant residues (the same for all proteins in the library) and variable residues (which vary from member to member). This is simply because, for a given range of variation at each position, the sequence space (simple diversity) grows exponentially with the number of residue positions, so at some point it becomes inconvenient for all residues of a peptide to be variable positions. Since proteins are usually larger than oligopeptides, it is more common for protein libraries than oligopeptide libraries to feature variable positions.

In the case of a protein library, it is desirable to focus the mutations at those sites which are tolerant of mutation. These may be determined by alanine scanning mutagenesis or by comparison of the protein sequence to that of homologous proteins of similar activity. It is also more likely that mutation of surface residues will directly affect binding. Surface residues may be determined by inspecting a 3D structure of the protein, or by labeling the surface and then ascertaining which residues have received labels. They may also be inferred by identifying regions of high hydrophilicity within the protein.

Because proteins are often altered at some sites but not others, protein libraries can be considered a special case of the biased peptide library.

There are several reasons that one might screen a protein library instead of an oligopeptide library, including (1) a particular protein, mutated in the library, has the desired activity to some degree already, and (2) the oligopeptides are not expected to have a sufficiently high affinity or specificity since they do not have a stable conformation.

When the protein library is based on a parental protein which does not have the desired activity, the parental protein will usually be one which is of high stability (melting point >=50 deg. C.) and/or possessed of hypervariable regions.

The variable domains of an antibody possess hypervariable regions and hence, in some embodiments, the protein library comprises members which comprise a mutant of VH or VL chain, or a mutant of an antigen-specific binding fragment of such a chain. VH and VL chains are usually each about 110 amino acid residues, and are held in proximity by a disulfide bond between the adjoing CL and CH1 regions to form a variable domain. Together, the VH, VL, CL and CH1 form an Fab fragment.

In human heavy chains, the hypervariable regions are at 31-35, 49-65, 98-111 and 84-88, but only the first three are involved in antigen binding. There is variation among VH and VL chains at residues outside the hypervariable regions, but to a much lesser degree.

A sequence is considered a mutant of a VH or VL chain if it is at least 80% identical to a naturally occurring VH or VL chain at all residues outside the hypervariable region.

In a preferred embodiment, such antibody library members comprise both at least one VH chain and at least one VL chain, at least one of which is a mutant chain, and which chains may be derived from the same or different antibodies. The VH and VL chains may be covalently joined by a suitable linker moiety, as in a “single chain antibody”, or they may be noncovalently joined, as in a naturally occurring variable domain.

If the joining is noncovalent, and the library is displayed on cells or virus, then either the VH or the VL chain may be fused to the carrier surface/coat protein. The complementary chain may be co-expressed, or added exogenously to the library.

The members may further comprise some or all of an antibody constant heavy and/or constant light chain, or a mutant thereof.

Peptoid Library

A peptoid is an analogue of a peptide in which one or more of the peptide bonds (—NH—CO—) are replaced by pseudopeptide bonds, which may be the same or different. It is not necessary that all of the peptide bonds be replaced, i.e., a peptoid may include one or more conventional amino acid residues, e.g., proline.

A peptide bond has two small divalent linker elements, —NH— and —CO—. Thus, a preferred class of psuedopeptide bonds are those which consist of two small divalent linker elements. Each may be chosen independently from the group consisting of amine (—NH—), substituted amine (—NR—), carbonyl (—CO—), thiocarbonyl (—CS—), methylene (—CH2-), monosubstituted methylene (—CHR—), disubstituted methylene (—CR1R2-), ether (—O—) and thioether (—S—). The more preferred pseudopeptide bonds include:
N-modified —NRCO—
Carba Ψ —CH, —CH₂—
Depsi Ψ —CO—O—
Hydroxyethylene Ψ —CHOH—CH₂—
Ketomethylene Ψ —CO—CH₂—
Methylene-Oxy —CH₂—O—
Reduced —CH₂—NH—
Thiomethylene —CH, —S—
Thiopeptide —CS—NH—
Retro-Inverso —CO—NH—

A single peptoid molecule may include more than one kind of pseudopeptide bond.

For the purposes of introducing diversity into a peptoid library, one may vary (1) the side chains attached to the core main chain atoms of the monomers linked by the pseudopeptide bonds, and/or (2) the side chains (e.g., the —R of an —NRCO—) of the pseudopeptide bonds. Thus, in one embodiment, the monomeric units which are not amino acid residues are of the structure —NR1—CR2-CO—, where at least one of R1 and R2 are not hydrogen. If there is variability in the pseudopeptide bond, this is most conveniently done by using an —NRCO— or other pseudopeptide bond with an R group, and varying the R group. In this event, the R group will usually be any of the side chains characterizing the amino acids of peptides, as previously discussed.

If the R group of the pseudopeptide bond is not variable, it will usually be small, e.g., not more than 10 atoms (e.g., hydroxyl, amino, carboxyl, methyl, ethyl, propyl).

If the conjugation chemistries are compatible, a simple combinatorial library may include both peptides and peptoids.

Peptide Nucleic Acid Library

A PNA oligomer is here defined as one comprising a plurality of units, at least one of which is a PNA monomer which comprises a side chain comprising a nucleobase. For nucleobases, see U.S. Pat. No. 6,077,835.

The classic PNA oligomer is composed of (2-aminoethyl)glycine units, with nucleobases attached by methylene carbonyl linkers. That is, it has the structure
H—(—HN—CH₂—CH₂—N(—CO—CH₂—B)—CH₂—CO—)ₙ—OH
where the outer parenthesized substructure is the PNA monomer.

In this structure, the nucleobase B is separated from the backbone N by three bonds, and the points of attachment of the side chains are separated by six bonds. The nucleobase may be any of the bases included in the nucleotides discussed in connection with oligonucleotide libraries. The bases of nucleotides A, G, T, C and U are preferred.

A PNA oligomer may further comprise one or more amino acid residues, especially glycine and proline.

One can readily envision related molecules in which (1) the —COCH2- linker is replaced by another linker, especially one composed of two small divalent linkers as defined previously, (2) a side chain is attached to one of the three main chain carbons not participating in the peptide bond (either instead or in addition to the side chain attached to the N of the classic PNA); and/or (3) the peptide bonds are replaced by pseudopeptide bonds as disclosed previously in the context of peptoids.

PNA oligomer libraries have been made; see e.g. Cook, U.S. Pat. No. 6,204,326.

Small Organic Compound Library

The small organic compound library (“compound library”, for short) is a combinatorial library whose members are suitable for use as drugs if, indeed, they have the ability to mediate a biological activity of the target protein.

Peptides have certain disadvantages as drugs. These include susceptibility to degradation by serum proteases, and difficulty in penetrating cell membranes. Preferably, all or most of the compounds of the compound library avoid, or at least do not suffer to the same degree, one or more of the pharmaceutical disadvantages of peptides.

In designing a compound library, it is helpful to bear in mind the methods of molecular modification typically used to obtain new drugs. Three basic kinds of modification may be identified: disjunction, in which a lead drug is simplified to identify its component pharmacophoric moieties; conjunction, in which two or more known pharmacophoric moieties, which may be the same or different, are associated, covalently or noncovalently, to form a new drug; and alteration, in which one moiety is replaced by another which may be similar or different, but which is not in effect a disjunction or conjunction. The use of the terms “disjunction”, “conjunction” and “alteration” is intended only to connote the structural relationship of the end product to the original leads, and not how the new drugs are actually synthesized, although it is possible that the two are the same.

The process of disjunction is illustrated by the evolution of neostigmine (1931) and edrophonium (1952) from physostigmine (1925). Subsequent conjunction is illustrated by demecarium (1956) and ambenonium (1956).

Alterations may modify the size, polarity, or electron distribution of an original moiety. Alterations include ring closing or opening, formation of lower or higher homologues, introduction or saturation of double bonds, introduction of optically active centers, introduction, removal or replacement of bulky groups, isosteric or bioisosteric substitution, changes in the position or orientation of a group, introduction of alkylating groups, and introduction, removal or replacement of groups with a view toward inhibiting or promoting inductive (electrostatic) or conjugative (resonance) effects.

Thus, the substituents may include electron acceptors and/or electron donors. Typical electron donors (+I) include —CH₃, —CH₂R, —CHR₂, —CR₃ and —COO—. Typical electron acceptors (−I) include —NH₃+, —NR₃+, —NO₂, —CN, —COOH, —COOR, —CHO, —COR, —COR, —F, —Cl, —Br, —OH, —OR, —SH, —SR, —CH═CH₂, —CR═CR₂, and —C═CH.

The substituents may also include those which increase or decrease electronic density in conjugated systems. The former (+R) groups include —CH₃, —CR₃, —F, —Cl, —Br, —I, —OH, —OR, —OCOR, —SH, —SR, —NH₂, —NR₂, and —NHCOR. The later (—R) groups include —NO₂, —CN, —CHC, —COR, —COOH, —COOR, —CONH₂, —SO₂R and —CF₃.

Synthetically speaking, the modifications may be achieved by a variety of unit processes, including nucleophilic and electrophilic substitution, reduction and oxidation, addition elimination, double bond cleavage, and cyclization.

For the purpose of constructing a library, a compound, or a family of compounds, having one or more pharmacological activities (which need not be related to the known or suspected activities of the target protein), may be disjoined into two or more known or potential pharmacophoric moieties. Analogues of each of these moieties may be identified, and mixtures of these analogues reacted so as to reassemble compounds which have some similarity to the original lead compound. It is not necessary that all members of the library possess moieties analogous to all of the moieties of the lead compound.

The design of a library may be illustrated by the example of the benzodiazepines. Several benzodiazepine drugs, including chlordiazepoxide, diazepam and oxazepam, have been used as anti-anxiety drugs. Derivatives of benzodiazepines have widespread biological activities; derivatives have been reported to act not only as anxiolytics, but also as anticonvulsants; cholecystokinin (CCK) receptor subtype A or B, kappa opioid receptor, platelet activating factor, and HIV transactivator Tat antagonists, and GPIIbIIa, reverse transcriptase and ras farnesyltransferase inhibitors.

The benzodiazepine structure has been disjoined into a 2-aminobenzophenone, an amino acid, and an alkylating agent. See Bunin, et al., Proc. Nat. Acad. Sci. USA, 91:47O8 (1994). Since only a few 2-aminobenzophenone derivatives are commercially available, it was later disjoined into 2-aminoarylstannane, an acid chloride, an amino acid, and an alkylating agent. Bunin, et al., Meth. Enzymol., 267:448 (1996). The arylstannane may be considered the core structure upon which the other moieties are substituted, or all four may be considered equals which are conjoined to make each library member.

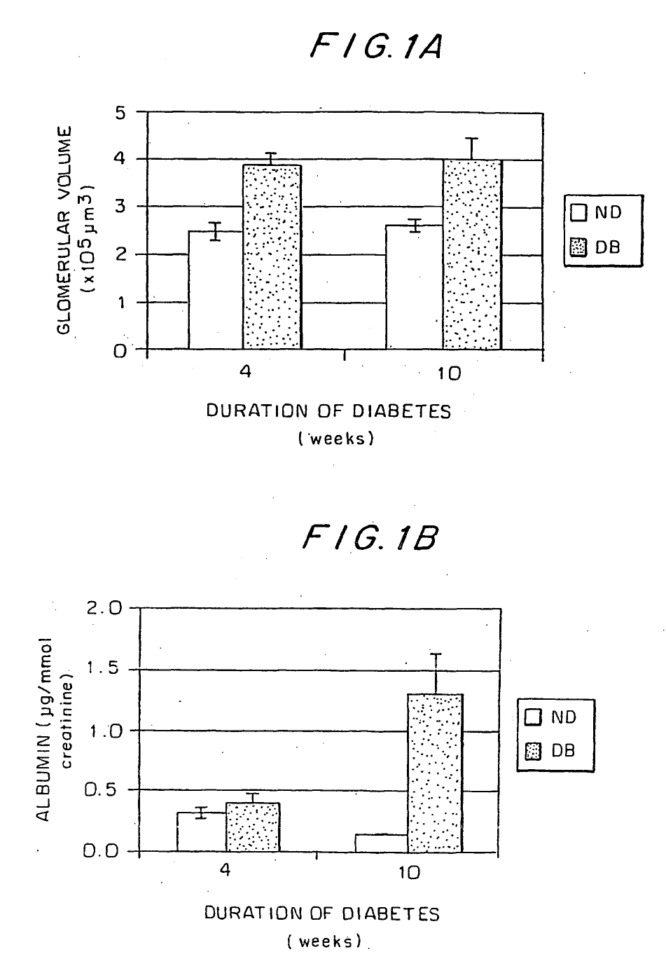

A basic library synthesis plan and member structure is shown in FIG. 1 of Fowlkes, et al., U.S. Ser. No. 08/740,671, incorporated by reference in its entirety. The acid chloride building block introduces variability at the R¹ site. The R² site is introduced by the amino acid, and the R³ site by the alkylating agent. The R⁴ site is inherent in the arylstannane. Bunin, et al. generated a 1,4-benzodiazepine library of 11,200 different derivatives prepared from 20 acid chlorides, 35 amino acids, and 16 alkylating agents. (No diversity was introduced at R⁴; this group was used to couple the molecule to a solid phase.) According to the Available Chemicals Directory (HDL Information Systems, San Leandro Calif.), over 300 acid chlorides, 80 Fmoc-protected amino acids and 800 alkylating agents were available for purchase (and more, of course, could be synthesized). The particular moieties used were chosen to maximize structural dispersion, while limiting the numbers to those conveniently synthesized in the wells of a microtiter plate. In choosing between structurally similar compounds, preference was given to the least substituted compound.

The variable elements included both aliphatic and aromatic groups. Among the aliphatic groups, both acyclic and cyclic (mono- or poly-) structures, substituted or not, were tested. (While all of the acyclic groups were linear, it would have been feasible to introduce a branched aliphatic). The aromatic groups featured either single and multiple rings, fused or not, substituted or not, and with heteroatoms or not. The secondary substitutents included —NH₂, —OH, —OMe, —CN, —Cl, —F, and —COOH. While not used, spacer moieties, such as —O—, —S—, —OO—, —CS—, —NH—, and —NR—, could have been incorporated.

Bunin et al. suggest that instead of using a 1,4-benzodiazepine as a core structure, one may instead use a 1,4-benzodiazepine-2,5-dione structure.

As noted by Bunin et al., it is advantageous, although not necessary, to use a linkage strategy which leaves no trace of the linking functionality, as this permits construction of a more diverse library.

Other combinatorial nonoligomeric compound libraries known or suggested in the art have been based on carbamates, mercaptoacylated pyrrolidines, phenolic agents, aminimides, N-acylamino ethers (made from amino alcohols, aromatic hydroxy acids, and carboxylic acids), N-alkylamino ethers (made from aromatic hydroxy acids, amino alcohols and aldehydes) 1,4-piperazines, and 1,4-piperazine-6-ones.

DeWitt, et al., Proc. Nat. Acad. Sci. (USA), 90:6909-13 (1993) describe the simultaneous but separate, synthesis of 40 discrete hydantoins and 40 discrete benzodiazepines. They carry out their synthesis on a solid support (inside a gas dispersion tube), in an array format, as opposed to other conventional simultaneous synthesis techniques (e.g., in a well, or on a pin). The hydantoins were synthesized by first simultaneously deprotecting and then treating each of five amino acid resins with each of eight isocyanates. The benzodiazepines were synthesized by treating each of five deprotected amino acid resins with each of eight 2-amino benzophenone imines.

Chen, et al., J. Am. Chem. Soc., 116:2661-62 (1994) described the preparation of a pilot (9 member) combinatorial library of formate esters. A polymer bead-bound aldehyde preparation was “split” into three aliquots, each reacted with one of three different ylide reagents. The reaction products were combined, and then divided into three new aliquots, each of which was reacted with a different Michael donor. Compound identity was found to be determinable on a single bead basis by gas chromatography/mass spectroscopy analysis.

Holmes, U.S. Pat. No. 5,549,974 (1996) sets forth methodologies for the combinatorial synthesis of libraries of thiazolidinones and metathiazanones. These libraries are made by combination of amines, carbonyl compounds, and thiols under cyclization conditions.

Ellman, U.S. Pat. No. 5,545,568 (1996) describes combinatorial synthesis of benzodiazepines, prostagiandins, beta-turn mimetics, and glycerol-based compounds. See also Ellman, U.S. Pat. No. 5,288,514.

Summerton, U.S. Pat. No. 5,506,337 (1996) discloses methods of preparing a combinatorial library formed predominantly of morpholino subunit structures.

Heterocylic combinatorial libraries are reviewed generally in Nefzi; et al.; Chem. Rev., 97:449-472 (1997).

One or more moieties of the following types may be incorporated into compounds of the library, as many drugs fall into one or more of the following categories:

acetals

acids

alcohols

amides

amidines

amines

amino acids

amino alcohols

amino ethers

amino ketenes

ammonium compounds

azo compounds

enols

esters

ethers

glycosides

guanidines

halogenated compounds

hydrocarbons

ketones

lactams

lactones

mustards

nitro compounds

nitroso compounds

phenones

quinones

semicarbazones

stilbenes

sulfonamides

sulfones

thiols

thioamides

thioureas

ureas

ureides

urethans

Without attempting to exhaustively recite all pharmacological classes of drugs, or all drug structures, one or more compounds of the chemical structures listed below have been found to exhibit the indicated pharmacological activity, and these structures, or derivatives, may be used as design elements in screening fox further compounds of the same or different activity. (In some cases, one or more lead drugs of the class are indicated.)

hypnotics
higher alcohols (clomethiazole)
aldehydes (chloral hydrate)
carbamates (meprobamate)
acyclic ureides (acetylcarbromal)
barbiturates (barbital)
benzodiazepine (diazepam)

anticonvulsants
barbiturates (phenobarbital)
hydantoins (phenytoin)
oxazolidinediones (trimethadione)
succinimides (phensuximide)
acylureides (phenacemides)

narcotic analgesics
morphines
phenylpiperidines (meperidine)
diphenylpropylamines (methadone)
phenothiazines (methotrimeprazine)

analgesics, antipyretics, antirheumatics
salicylates (acetylsalicylic acid)
p-aminophenol (acetaminophen)
5-pyrazolone (dipyrone)
3,5-pyrazolidinedione (phenylbutazone)
arylacetic acid (indomethacin)
adrenocortical steroids (cortisone, dexamethasone,
prednisone, triamcilone)
athranilic acids

neuroleptics
phenothiazine (chlorpromazine)
thioxanthene (chlorprothixene)
reserpine
butyrophenone (halopendol)

anxiolytics
propandiol carbamates (meprobamate)
benzodiazepines (chlordiazepoxide, diazepam, oxazepam)

antidipressants
tricyclics (imipramine)

muscle/relaxants
propanediols and carbamates (mephenesin)

CNS stimulants
xanthines (caffeine, theophylline)
phenylalkylamines (amphetamine)
(Fenetylline is a conjunction of theophylline and amphetamine)
oxazolidinones (pemoline)

cholinergics
choline esters (acetylcholine)
N,N-dimethylcarbamates

adrenergics
aromatic amines (epinephrine, isoproterenol, phenylephrine)
alicyclic amines (cyclopentamine)
aliphatic amines (methylhexaneamine)
imidazolines (naphazoline)

anti-adrenergics
indolethylamine alkaloids (dihydroergotamine)
imidazoles (tolazoline)
benzodioxans (piperoxan)
beta-haloalkylamines (phenoxybenzamine)
dibenzazepines (azapetine)
hydrazinophthalazines (hydralazine)

antihistamines
ethanolamines (diphenhydramine)
ethylenediamines (tripelennomine)
alkylamines (chlorpheniramine)
piperazines (cyclizine)
phenothiazines (promethazine)

local anesthetics
benzoic acid
esters (procaine, isobucaine, cyclomethycaine)
basic amides (dibucaine)
anilides, toluidides, 2,6-xylidides (lidocaine)
tertiary amides (oxetacaine)

vasodilators
polyol nitrates (nitroglycerin)

diuretics
xanthines
thiazides (chlorothiazide)
sulfonamides (chlorthalidone)

antihelmintics
cyanine dyes

antimalarials
4-aminoquinolines
8-aminoquinolines
pyrimidines
biguanides
acridines
dihydrotriazines
sulfonamides
sulfones

antibacterials
antibiotics
   penicillins
   cephalosporins
   octahydronapthacenes (tetracycline)
sulfonamides
nitrofurans
cyclic amines
naphthyridines
xylenols

antitumor
alkylating agents
   nitrogen mustards
   aziridines
   methanesulfonate esters
   epoxides
amino acid antagonists
folic acid antagonists
pyrimidine antagonists
purine antagonists

antiviral
adamantanes
nucleosides
thiosemicarbazones
inosines
amidines and guanidines
isoquinolines
benzimidazoles
piperazines

For pharmacological classes, see, e.g., Goth, Medical Pharmacology: Principles and Concepts (C.V. Mosby Co.: 8th ed. 1976); Korolkovas and Burckhalter, Essentials of Medicinal Chemistry (John Wiley & Sons, Inc.: 1976). For synthetic methods, see, e.g., Warren, Organic Synthesis: The Disconnection Approach (John Wiley & Sons, Ltd.: 1982); Fuson, Reactions of Organic Compounds (John Wiley & Sons: 1966); Payne and Payne, How to do an Organic Synthesis (Allyn and Bacon, Inc.: 1969); Greene, Protective Groups in Organic Synthesis (Wiley-Interscience). For selection of substituents, see e.g., Hansch and Leo, Substituent Constants for Correlation Analysis in Chemistry and Biology (John Wiley & Sons: 1979).

The library is preferably synthesized so that the individual members remain identifiable so that, if a member is shown to be active, it is not necessary to analyze it. Several methods of identification have been proposed, including:
(1) encoding, i.e., the attachment to each member of an identifier moiety which is more readily identified than the member proper. This has the disadvantage that the tag may itself influence the activity of the conjugate.
(2) spatial addressing, e.g., each member is synthesized only at a particular coordinate on or in a matrix, or in a particular chamber. This might be, for example, the location of a particular pin, or a particular well on a microtiter plate, or inside a “tea bag”.
   The present invention is not limited to any particular form of identification.

However, it is possible to simply characterize those members of the library which are found to be active, based on the characteristic spectroscopic indicia of the various building blocks.

Solid phase synthesis permits greater control over which derivatives are formed. However, the solid phase could interfere with activity. To overcome this problem, some or all of the molecules of each member could be liberated, after synthesis but before screening.

Examples of candidate simple libraries which might be evaluated include derivatives of the following:

Cyclic Compounds Containing One Hetero Atom
Heteronitrogen
   pyrroles
      pentasubstituted pyrroles
   pyrrolidines
   pyrrolines
   prolines
   indoles
   beta-carbolines
   pyridines
      dihydropyridines
      1,4-dihydropyridines
      pyrido[2,3-d]pyrimidines
      tetrahydro-3H-imidazo[4,5-c]pyridines
   Isoquinolines
      tetrahydroisoquinolines
   quinolones
   beta-lactams
      azabicyclo[4.3.0]nonen-8-one amino acid
Heterooxygen
   furans
      tetrahydrofurans
         2,5-disubstituted tetrahydrofurans
   pyrans
      hydroxypyranones
      tetrahydroxypyranones
   gamma-butyrolactones
Heterosulfur
   sulfolenes

Cyclic Compounds with Two or More Hetero Atoms
Multiple heteronitrogens
   imidazoles
   pyrazoles
   piperazines
      diketopiperazines
      arylpiperazines
      benzylpiperazines
   benzodiazepines
   1,4-benzodiazepine-2,5-diones
   hydantoins
      5-alkoxyhydantoins
   dihydropyrimidines
   1,3-disubstituted-5,6-dihydopyrimidine-2,4-diones
   cyclic ureas
   cyclic thioureas
   quinazolines
      chiral 3-substituted-quinazoline-2,4-diones
   triazoles
      1,2,3-triazoles
   purines
Heteronitrogen and Heterooxygen
   dikelomorpholines
   isoxazoles
   isoxazolines
Heteronitrogen and Heterosulfur
   thiazolidines
      N-axylthiazolidines
   dihydrothiazoles
      2-methylene-2,3-dihydrothiazates
      2-aminothiazoles
   thiophenes
      3-amino thiophenes
   4-thiazolidinones
   4-melathiazanones
   benzisothiazolones

For details on synthesis of libraries, see Nefzi, et al., Chem. Rev., 97:449-72 (1997), and references cited therein.

Pharmaceutical Methods and Preparations

The preferred animal subject of the present invention is a mammal. By the term “mammal” is meant an individual belonging to the class Mammalia. The invention is particularly useful in the treatment of human subjects, although it is intended for veterinary and nutritional uses as well. Preferred nonhuman subjects are of the orders Primata (e.g., apes and monkeys), Artiodactyla or Perissodactyla (e.g., cows, pigs, sheep, horses, goats), Carnivora (e.g., cats, dogs), Rodenta (e.g., rats, mice, guinea pigs, hamsters), Lagomorpha (e.g., rabbits) or other pet, farm or laboratory mammals.

The term “protection”, as used herein, is intended to include “prevention,” “suppression” and “treatment.” “Prevention”, strictly speaking, involves administration of the pharmaceutical prior to the induction of the disease. (or other adverse clinical condition). “Suppression” involves administration of the composition prior to the clinical appearance of the disease. “Treatment” involves administration of the protective composition after the appearance of the disease.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, unless qualified, the term “prevention” will be understood to refer to both prevention in the strict sense, and to suppression.

The preventative or prophylactic use of a pharmaceutical involves identifying subjects who are at higher risk than the general population of contracting the disease, and administering the pharmaceutical to them in advance of the clinical appearance of the disease. The effectiveness of such use is measured by comparing the subsequent incidence or severity of the disease, or of particular symptoms of the disease, in the treated subjects against that in untreated subjects of the same high risk group.

While high risk factors vary from disease to disease, in general, these include (1) prior occurrence of the disease in one or more members of the same family, or, in the case of a contagious disease, in individuals with whom the subject has come into potentially contagious contact at a time when the earlier victim was likely to be contagious, (2) a prior occurrence of the disease in the subject, (3) prior occurrence of a related disease, or a condition known to increase the likelihood of the disease, in the subject; (4) appearance of a suspicious level of a marker of the disease, or a related disease or condition; (5) a subject who is immunologically compromised, e.g., by radiation treatment, HIV infection, drug use, etc., or (6) membership in a particular group (e.g., a particular age, sex, race, ethnic group, etc.) which has been epidemiologically associated with that disease.

A prophylaxis or treatment may be curative, that is, directed at the underlying cause of a disease, or ameliorative, that is, directed at the symptoms of the disease, especially those which reduce the quality of life.

It should also be understood that to be useful, the protection provided need not be absolute, provided that it is sufficient to carry clinical value. An agent which provides protection to a lesser degree than do competitive agents may still be of value if the other agents are ineffective for a particular individual, if it can be used in combination with other agents to enhance the level of protection, or if it is safer than competitive agents. It is desirable that there be a statistically significant (p=0.05 or less) improvement in the treated subject relative to an appropriate untreated control, and it is desirable that this improvement be at least 10%, more preferably at least 25%, still more preferably at least 50%, even more preferably at least 100%, in some indicia of the incidence or severity of the disease or of at least one symptom of the disease.

At least one of the drugs of the present invention may be administered, by any means that achieve their intended purpose, to protect a subject against a disease or other adverse condition. The form of administration may be systemic or topical. For example, administration of such a composition may be by various parenteral routes such as subcutaneous, intravenous, intradermal, intramuscular, intraperitoneal, intranasal, transdermal, or buccal routes. Alternatively, or concurrently, administration may be by the oral route. Parenteral administration can be by bolus injection or by gradual perfusion over time.

A typical regimen comprises administration of an effective amount of the drug, administered over a period ranging from a single dose, to dosing over a period of hours, days, weeks, months, or years.

It is understood that the suitable dosage of a drug of the present invention will be dependent upon the age, sex, health, and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This will typically involve adjustment of a standard dose, e.g., reduction of the dose if the patient has a low body weight.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The total dose required for each treatment may be administered by multiple doses or in a single dose. The protein may be administered alone or in conjunction with other therapeutics directed to the disease or directed to other symptoms thereof.

The appropriate dosage form will depend on the disease, the pharmaceutical, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

In the case of peptide drugs, the drug may be administered in the form of an expression vector comprising a nucleic acid encoding the peptide; such a vector, after in corporation into the genetic complement of a cell of the patient, directs synthesis of the peptide. Suitable vectors include genetically engineered poxviruses (vaccinia), adenoviruses, adeno-associated viruses, herpesviruses and lentiviruses which are or have been rendered nonpathogenic.

In addition to at least one drug as described herein, a pharmaceutical composition may contain suitable pharmaceutically acceptable carriers, such as excipients, carriers and/or auxiliaries which facilitate processing of the active compounds into preparations which can be used pharmaceutically. See, e.g., Berker, supra, Goodman, supra, Avery, supra and Ebadi, supra, which are entirely incorporated herein by reference, included all references cited therein.

Assay Compositions and Methods

Target Organism

The invention contemplates that it may be appropriate to ascertain or to mediate the biological activity of a substance of this invention in a target organism.

The target organism may be a plant, animal, or microorganism.

In the case of a plant, it may be an economic plant, in which case the drug may be intended to increase the disease, weather or pest resistance, alter the growth characteristics, or otherwise improve the useful characteristics or mute undesirable characteristics of the plant. Or it may be a weed in which case the drug may be intended to kill or otherwise inhibit the growth of the plant, or to alter its characteristics to convert it from a weed to an economic plant. The plant may be a tree, shrub, crop, grass, etc. The plant may be an algae (which are in some cases also microorganisms), or a vascular plant, especially gymnosperms (particularly conifers) and angiosperms. Angiosperms may be monocots or dicots. The plants of greatest interest are rice, wheat, corn, alfalfa, soybeans, potatoes, peanuts, tomatoes, melons, apples, pears, plums, pineapples, fir, spruce, pine, cedar, and oak. If the target organism is a microorganism, it may be algae, bacteria, fungi, or a virus (although the biological activity of a virus must be determined in a virus-infected cell). The microorganism may be human or other animal or plant pathogen, or it may be nonpathogenic. It may be a soil or water organism, or one which normally lives inside other living things.

If the target organism is an animal, it may be a vertebrate or a nonvertebrate animal. Nonvertebrate animals are chiefly of interest when they act as pathogens or parasites, and the drugs are intended to act as biocidic or biostatic agents. Nonvertebrate animals of interest include worms, mollusks, and arthropods.

The target, organism may also be a vertebrate animal, i.e., a mammal, bird, reptile, fish or amphibian. Among mammals, the target animal preferably belongs to the order Primata (humans, apes and monkeys), Artiodactyla (e.g., cows, pigs, sheep, goats, horses), Rodenta (e.g., mice, rats) Lagomorpha (e.g., rabbits, hares), or Carnivora (e.g., cats, dogs). Among birds, the target animals are preferably of the orders Anseriformes (e.g., ducks, geese, swans) or Galliformes (e.g., quails, grouse, pheasants, turkeys and chickens). Among fish, the target animal is preferably of the order Clupeiformes (e.g., sardines, shad, anchovies, whitefish, salmon).

Target Tissues

The term “target tissue” refers to any whole animal, physiological system, whole organ, part of organ, miscellaneous tissue, cell, or cell component (e.g., the cell membrane) of a target animal in which biological activity may be measured.

Routinely in mammals one would choose to compare and contrast the biological impact on virtually any and all tissues which express the subject receptor protein. The main tissues to use are: brain, heart, lung, kidney, liver, pancreas, skin, intestines, adipose, stomach, skeletal muscle, adrenal glands, breast, prostate, vasculature, retina, cornea, thyroid gland, parathyroid glands, thymus, bone marrow, bone, etc.

Another classification would be by cell type: B cells, T cells, macrophages, neutrophils, eosinophils, mast cells, platelets, megakaryocytes, erythrocytes, bone marrow stomal cells, fibroblasts, neurons, astrocytes, neurolia, microglia, epithelial cells (from any organ, e.g. skin, breast, prostate, lung, intestines etc), cardiac muscle cells, smooth muscle cells, striated muscle cells, osteoblasts, osteocytes, chondroblasts, chondrocytes, keratinocytes, melanocytes, etc.

Of course, in the case of a unicellular organism, there is no distinction between the “target organism” and the “target tissue”.

Screening Assays

Assays intended to determine the binding or the biological activity of a substance are called preliminary screening assays.

Screening assays will typically be either in vitro (cell-free) assays (for binding to an immobilized receptor) or cell-based assays (for alterations in the phenotype of the cell). They will not involve screening of whole multicellular organisms, or isolated-organs. The comments on diagnostic biological assays apply mutatis mutandis to screening cell-based assays.

In Vitro vs. In Vivo Assays

The term in vivo is descriptive of ah event, such as binding or enzymatic action, which occurs within a living organism. The organism in question may, however, be genetically modified. The term in vitro refers to an event which occurs outside a living organism. Parts of an organism (e.g., a membrane, or an isolated biochemical) are used, together with artificial substrates and/or conditions. For the purpose of the present invention, the term in vitro excludes events occurring inside or on an intact cell, whether of a unicellular or multicellular organism.

In vivo assays include both cell-based assays, and organismic assays. The cell-based assays include both assays on unicellular organisms, and assays on isolated cells or cell cultures derived from multicellular organisms. The cell cultures may be mixed, provided that they are not organized into tissues or organs. The term organismic assay refers to assays on whole multicellular organisms, and assays on isolated organs or tissues of such organisms.

In Vitro Diagnostic Methods and Reagents

The in vitro assays of the present invention may be applied to any suitable analyte-containing sample, and may be qualitative or quantitative in nature.

Sample

The sample will normally be a biological fluid, such as blood, urine, lymph, semen, milk, or cerebrospinal fluid, or a fraction or derivative thereof, or a biological tissue, in the form of, e.g., a tissue section or homogenate. However, the sample conceivably could be (or derived from) a food or beverage, a pharmaceutical or diagnostic composition, soil, or surface or ground water. If a biological fluid or tissue, it may be taken from a human or other mammal, vertebrate or animal, or from a plant. The preferred sample is blood, or a fraction or derivative thereof.

Binding and Reaction Assays

The assay may be a binding assay, in which one step involves the binding of a diagnostic reagent to the analyte, or a reaction assay, which involves the reaction of a reagent with the analyte. The reagents used in a binding assay may be classified as to the nature of their interaction with analyte: (1) analyte analogues, or (2) analyte binding molecules (ABM). They may be labeled or insolubilized.

In a reaction assay, the assay may look for a direct reaction between the analyte and a reagent, which is reactive with the analyte, or if the analyte is an enzyme or enzyme inhibitor, for a reaction catalyzed or inhibited by the analyte. The reagent may be a reactant, a catalyst, or an inhibitor for the reaction.

An assay may involve a cascade of steps in which the product of one step acts as the target for the next step. These steps may be binding steps, reaction steps, or a combination thereof.

Signal Producing System (SPS)

In order to detect the presence, or measure the amount, of an analyte, the assay must provide for a signal producing system (SPS) in which there is a detectable difference in the signal produced, depending on whether the analyte is present or absent (or, in a quantitative assay, on the amount of the analyte). The detectable signal may be one which is visually detectable, or one detectable only with instruments. Possible signals include production of colored or luminescent products, alteration of the characteristics (including amplitude or polarization) of absorption or emission of radiation by an assay component or product, and precipitation or agglutination of a component or product. The term “signal” is intended to include the discontinuance of an existing signal, or a change in the rate of change of an observable parameter, rather than a change in its absolute value. The signal may be monitored manually or automatically.

In a reaction assay, the signal is often a product of the reaction. In a binding assay, it is normally provided by a label borne by a labeled reagent.

Labels

The component of the signal producing system which is most intimately associated with the diagnostic reagent is called the “label”. A label may be, e.g., a radioisotope, a fluorophore, an enzyme, a co-enzyme, an enzyme substrate, an electron-dense compound, an agglutinable particle.

The radioactive isotope can be detected by such means as the use of a gamma counter or a scintillation counter or by autoradiography. Isotopes which are particularly useful for the purpose of the present invention include ³H, ¹²⁵I, ¹³¹I, ³⁵S, ¹⁴C, ³²P and ³³P. ¹²⁵I is preferred for antibody labeling.

The label may also be a fluorophore. When the fluorescently labeled reagent is exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine.

Alternatively, fluorescence-emitting metals such as ¹²⁵Eu, or others of the lanthanide series, may be incorporate into a diagnostic reagent using such metal chelating groups as diethylenetriaminepentaacetic acid (DTPA) of ethylenediamine-tetraacetic acid (EDTA).

The label may also be a chemiluminescent compound. The presence of the chemiluminescently labeled reagent is then determined by detecting the presence of luminescence that arises during the course of a chemical reaction. Examples of particularly useful chemiluminescent labeling compounds are luminol, isolumino, theromatic acridinium ester, imidazole, acridinium salt and oxalate ester.

Likewise, a bioluminescent compound may be used for labeling. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Enzyme labels, such as horseradish peroxidase and alkaline phosphatase, are preferred. When an enzyme label is used, the signal producing system must also include a substrate for the enzyme. If the enzymatic reaction product is not itself detectable, the SPS will include one or more additional reactants so that a detectable product appears.

An enzyme analyte may act as its own label if an enzyme inhibitor is used as a diagnostic reagent.

Binding Assay Formats

Binding assays may be divided into two basic types, heterogeneous and homogeneous. In heterogeneous assays, the interaction between the affinity molecule and the analyte does not affect the label, hence, to determine the amount or presence of analyte, bound label must be separated from free label. In homogeneous assays, the interaction does affect the activity of the label, and therefore analyte levels can be deduced without the need for a separation step.

In one embodiment, the ABM is insolubilized by coupling it to a macromolecular support, and analyte in the sample is allowed to compete with a known quantity of a labeled or specifically labelable analyte analogue. The “analyte analogue” is a molecule capable of competing with analyte for binding to the ABM, and the term is intended to include analyte itself. It may be labeled already, or it may be labeled subsequently by specifically binding the label to a moiety differentiating the analyte analogue from analyte. The solid and liquid phases are separated, and the labeled analyte analogue in one phase is quantified. The higher the level of analyte analogue in the solid phase, i.e., sticking to the ABM, the lower the level of analyte in the sample.

In a “sandwich assay”, both an insolubilized ABM, and a labeled ABM are employed. The analyte is captured by the insolubilized ABM and is tagged by the labeled ABM, forming a ternary complex. The reagents may be added to the sample in either order, or simultaneously. The ABMs may be the same or different. The amount of labeled ABM in the ternary complex is directly proportional to the amount of analyte in the sample.

The two embodiments described above are both heterogeneous assays. However, homogeneous assays are conceivable. The key is that the label be affected by whether or not the complex is formed.

Conjugation Methods

A label may be conjugated, directly or indirectly (e.g., through a labeled anti-ABM antibody), covalently (e.g., Edith SPDP) or noncovalently, to the ABM, to produce a diagnostic reagent. Similarly, the ABM may be conjugated to a solid phase support to form a solid phase (“capture”) diagnostic reagent.

Suitable supports include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention.

The support material may have virtually any possible structural configuration so long as the coupled molecule is capable of binding to its target. Thus the support configuration may be spherical, as in a bead, or cylindrical, as in the inside surface of a test tube, or the external surface of a rod. Alternatively, the surface may be flat such as a sheet, test strip, etc.

Biolocical Assays

A biological assay measures or detects a biological response of a biological entity to a substance.

The biological entity may be a whole organism, an isolated organ or tissue, freshly isolated cells, an immortalized cell line, or a subcellular component (such as a membrane; this term should not be construed as including an isolated receptor). The entity may be, or may be derived from, an organism which occurs in nature, or which is modified in some way. Modifications may be genetic (including radiation and chemical mutants, and genetic engineering) or somatic (e.g., surgical, chemical, etc.). In the case of a multicellular entity, the modifications may affect some or all cells. The entity need not be the target organism, or a derivative thereof, if there is a reasonable correlation between bioassay activity in the assay entity and biological activity in the target organism.

The entity is placed in a particular environment, which may be more or less natural. For example, a culture medium may, but need not, contain serum or serum substitutes, and it may, but need not, include a support matrix of some kind, it may be still, or agitated. It may contain particular biological or chemical agents, or have particular physical parameters (e.g., temperature), that are intended to nourish or challenge the biological entity.

There must also be a detectable biological marker for the response. At the cellular level, the most common markers are cell survival and proliferation, cell behavior (clustering, motility), cell morphology (shape, color), and biochemical activity (overall DNA synthesis, overall protein synthesis, and specific metabolic activities, such: as utilization of particular nutrients, e.g. consumption of oxygen, production of CO₂, production of organic acids, uptake or discharge of ions).

The direct signal produced by the biological marker may be transformed by a signal producing system into a different signal which is more observable, for example, a fluorescent or colorimetric signal.

The entity, environment, marker and signal producing system are chosen to achieve a clinically acceptable level of sensitivity, specificity and accuracy.

In some cases, the goal will be to identify substances which mediate the biological activity of a natural biological entity, and the assay is carried out directly with that entity. In other cases, the biological entity is used simply as a model of some more complex (or otherwise inconvenient to work with) biological entity in that event, the model biological entity is used because activity in the model system is considered more predictive of activity in the ultimate natural biological entity than is simple binding activity in an in vitro system. The model entity is used instead of the ultimate entity because the former is more expensive or slower to work with, or because ethical considerations forbid working with the ultimate entity yet.

The model entity may be naturally occurring, if the model entity usefully models the ultimate entity under some conditions. Or it may be non-naturally occurring, with modifications that increase its resemblance to the ultimate entity.

Transgenic animals, such as transgenic mice, rats, and rabbits, have been found useful as model systems.

In cell-based model assays, where the biological activity is mediated by binding to a receptor (target protein), the receptor may be functionally connected to a signal (biological marker) producing system, which may be endogenous or exogenous to the cell. There are a number of techniques of doing this.

“Zero-Hybrid” Systems

In these systems, the binding of a peptide to the target protein results in a screenable or selectable phenotypic change, without resort to fusing the target protein (or a ligand binding moiety thereof) to an endogenous protein. It may be that the target protein is endogenous to the host cell, or is substantially identical to an endogenous receptor so that it can take advantage of the latter's native signal transduction pathway. Or sufficient elements of the signal transduction pathway normally associated with the target protein may be engineered into the cell so that the cell signals binding to the target protein.

“One-Hybrid” Systems

In these systems, a chimera receptor, a hybrid of the target protein and an endogenous receptor, is used. The chimeric receptor has the ligand binding characteristics of the target protein and the signal transduction characteristics of the endogenous receptor. Thus, the normal signal transduction pathway of the endogenous receptor is subverted.

Preferably, the endogenous receptor is inactivated, or the conditions of the assay avoid activation of the endogenous receptor, to improve the signal-to-noise ratio.

See Fowlkes U.S. Pat. No. 5,789,184 for a yeast system.

Another type of “one-hybrid” system combines a peptide: DNA-binding domain fusion with an unfused target receptor that possesses an activation domain.

“Two-Hybrid” System

In a preferred embodiment, the cell-based assay is a two hybrid system. This term implies that the ligand is incorporated into a first hybrid protein, and the receptor into a second hybrid protein. The first hybrid also comprises component A of a signal generating system, and the second hybrid comprises component B of that system. Components A and B, by themselves, are insufficient to generate a signal. However, if the ligand binds the receptor, components A and B are brought into sufficiently close proximity so that they can cooperate to generate a signal.

Components A and B may naturally occur, or be substantially identical to moieties which naturally occur, as components of a single naturally occurring biomolecule, or they may naturally occur, or be substantially identical to moieties which naturally occur, as separate naturally occurring biomolecules which interact in nature.

Two-Hybrid System: Transcription Factor Type

In a preferred “two-hybrid” embodiment, one member of a peptide ligand:receptor binding pair is expressed as a fusion to a DNA-binding domain (DBD) from a transcription factor (this fusion protein is called the “bait”), and the other is expressed as a fusion to a transactivation domain (TAD) (this fusion protein is called the “fish”, the “prey”, or the “catch”). The transactivation domain should be complementary to the DNA-binding domain, i.e., it should interact with the latter so as to activate transcription of a specially designed reporter gene that carries a binding site for the DNA-binding domain. Naturally, the two fusion proteins must likewise be complementary.

This complementarity may be achieved by use of the complementary and separable DNA-binding and transcriptional activator domains of a single transcriptional activator protein, or one may use complementary domains derived from different proteins. The domains may be identical to the native domains, or mutants thereof. The assay members may be fused directly to the DBD or TAD, or fused through an intermediated linker.

The target DNA operator may be the native operator sequence, or a mutant operator. Mutations in the operator may be coordinated with mutations in the DBD and the TAD. An example of a suitable transcription activation system is one comprising the DNA-binding domain from the bacterial repressor LexA and the activation domain from the yeast transcription factor Gal4, with the reporter gene operably linked to the LexA operator.

It is not necessary to employ the intact target receptor; just the ligand-binding moiety is sufficient.

The two fusion proteins may be expressed from the same or different vectors. Likewise, the activatable reporter gene may be expressed from the same vector as either fusion protein (or both proteins), or from a third vector.

Potential DNA-binding domains include Gal4, LexA, and mutant domains substantially identical to the above.

Potential activation domains include E. coli B42, Gal4 activation domain II, and HSV VP16, and mutant domains substantially identical to the above.

Potential operators include the native operators for the desired activation domain, and mutant domains substantially identical to the native operator.

The fusion proteins may comprise nuclear localization signals.

The assay system will include a signal producing system, too. The first element of this system is a reporter gene operably linked to an operator responsive to the DBD and TAD of choice. The expression of this reporter gene will result, directly or indirectly, in a selectable or screenable phenotype (the signal). The signal producing system may include, besides the reporter gene, additional genetic or biochemical elements which cooperate in the production of the signal. Such an element could be, for example, a selective agent in the cell growth medium. There may be more than one signal producing system, and the system may include more than one reporter gene.

The sensitivity of the system may be adjusted by, e.g., use of competitive inhibitors of any step in the activation or signal production process, increasing or decreasing the number of operators, using a stronger or weaker DBD or TAD, etc.

When the signal is the death or survival of the cell in question, or proliferation or nonproliferation of the cell in question, the assay is said to be a selection. When the signal merely results in a detectable phenotype by which the signalling cell may be differentiated from the same cell in a nonsignalling state (either way being a living cell), the assay is a screen. However, the term “screening assay” may be used in a broader sense to include a selection. When the narrower sense is intended, we will use the term “nonselective screen”.

Various screening and selection systems are discussed in Ladner, U.S. Pat. No. 5,198,346.

Screening and selection may be for or against the peptide: target protein or compound:target protein interaction.

Preferred assay cells are microbial (bacterial, yeast, algal, protozooal), invertebrate, vertebrate (esp. mammalian, particularly human). The best developed two-hybrid assays are yeast and mammalian systems.

Normally, two hybrid assays are used to determine whether a protein X and a protein Y interact, by virtue of their ability to reconstitute the interaction of the DBD and the TAD. However, augmented two-hybrid assays have been used to detect interactions that depend on a third, non-protein ligand.

For more guidance on two-hybrid assays, see Brent and Finley, Jr., Ann. Rev. Genet., 31:663-704 (1997); Fremont-Racine, et al., Nature Genetics, 277-281 (16 Jul. 1997); Allen, et al., TIBS, 511-16 (December 1995); LeCrenier, et al., BioEssays, 20:1-6 (1998); Xu, et al., Proc. Nat. Acad. sci. (USA), 94:12473-8 (November 1992); Esotak, et al., Mol. Cell. Biol., 15:5820-9 (1995); Yang, et al., Nucleic Acids Res., 23:1152-6 (1995); Bendixen, et al., Nucleic Acids Res., 22:1778-9 (1994); Fuller, et al., BioTechniques, 25:85-92 (July 1998); Cohen, et al., PNAS (USA) 95:14272-7 (1998); Kolonin and Finley, Jr., PNAS (USA) 95:14266-71 (1998). See also Vasavada, et al., PNAS (USA), 88:10686-90 (1991) (contingent replication assay), and Rehrauer, et al., J. Biol. Chem., 271:23865-73 91996) (LexA repressor cleavage assay).

Two-Hybrid Systems: Reporter Enzyme Type

In another embodiment, the components A and B reconstitute an enzyme which is not a transcription factor.

As in the last example, the effect of the reconstitution of the enzyme is a phenotypic change which may be a screenable change, a selectable change, or both.

In Vivo Diagnostic Uses

Radio-labelled ABM may be administered to the human or animal subject. Administration is typically by injection, e.g., intravenous or arterial or other means of administration in a quantity sufficient to permit subsequent dynamic and/or static imaging using suitable radio-detecting devices. The dosage is the smallest amount capable of providing a diagnostically effective image, and may be determined by means conventional in the art, using known radio-imaging agents as a guide.

Typically, the imaging is carried out on the whole body of the subject, or on that portion of the body or organ relevant to the condition or disease under study. The amount of radio-labelled ABM accumulated at a given point in time in relevant target organs can then be quantified.

A particularly suitable radio-detecting device is a scintillation camera, such as a gamma camera. A scintillation camera is a stationary device that can be used to image distribution of radio-labelled ABM. The detection device in the camera senses the radioactive decay, the distribution of which can be recorded. Data produced by the imaging system can be digitized. The digitized information can be analyzed over time discontinuously or continuously. The digitized data can be processed to produce images, called frames, of the pattern of uptake of the radio-labelled ABM in the target organ at a discrete point in time. In most continuous (dynamic) studies, quantitative data is obtained by observing changes in distributions of radioactive decay in target organs over time. In other words, a time-activity analysis of the data will illustrate uptake through clearance of the radio-labelled binding protein by the target organs with time.

Various factors should be taken into consideration in selecting an appropriate radioisotope. The radioisotope must be selected with a view to obtaining good quality resolution upon imaging, should be safe for diagnostic use in humans and animals, and should preferably have a short physical half-life so as to decrease the amount of radiation received by the body. The radioisotope used should preferably be pharmacologically inert, and, in the quantities administered, should not have any substantial physiological effect.

The ABM may be radio-labelled with different isotopes of iodine, for example ¹²³I, ¹²⁵I, or ¹³¹I (see for example, U.S. Pat. No. 4,609,725). The extent of radio-labeling must, however be monitored, since it will affect the calculations made based on the imaging results (i.e. a diiodinated ABM will result in twice the radiation count of a similar monoiodinated ABM over the same time frame).

In applications to human subjects, it may be desirable to use radioisotopes other than ¹²⁵I for labelling in order t decrease the total dosimetry exposure of the human body and to optimize the detectability of the labelled molecule (though this radioisotope can be used if circumstances require). Ready availability for clinical use is also a factor. Accordingly, for human applications, preferred radio-labels are for example, ^{99m}Tc, ⁶⁷Ga, ⁶⁸Ga, ⁹⁰Y, ¹¹¹In, ^{113m}In, ¹²³I, ¹⁸⁶Re, ¹⁸⁶Re or ²¹¹At.

The radio-labelled ABM may be prepared by various methods. These include radio-halogenation by the chloramine—T method or the lactoperoxidase method and subsequent purification by HPLC (high pressure liquid chromatography), for example as described by J. Gutkowska et al in “Endocrinology and Metabolism Clinics of America: (1987) 16 (1):183. Other known methods of radio-labelling can be used, such as IODOBEADS™.

There are a number of different methods of delivering the radio-labelled ABM to the end-user. It may be administered by any means that enables the active agent to reach the agent's site of action in the body of a mammal. Because proteins are subject to being digested when administered orally, parenteral administration, i.e., intravenous, subcutaneous, intramuscular, would ordinarily be used to optimize absorption of an ABM, such as an antibody, which is a protein.

### EXAMPLES

### Example 1

Differential Expression of Genes in Kidneys of Streptozotocin-Treated Mice as a Result of GHR/BP Knockout

To identify genes potentially involved in the protection of kidneys during diabetes, we induced diabetes by STZ treatment of GHR/BP −/− (knockout) mice (prepared as described by Zhou, et al., 1997, supra) and nontransgenic (+/+) mice. These mice had a 129 Ola/BalbC genetic background. A PCR-based strategy was used to generate cDNA subtraction libraries from RNA isolated from the kidneys of diabetic −/− and +/+ mice after ten weeks of hyperglycemia. To identify genes whose increased expression potentially results in protection against glomerulosclerosis, the cDNAs expressed in +/+ kidneys were subtracted from the cDNAs expressed in −/− kidneys, resulting in a library (−/−) consisting of cDNAs upregulated in the −/− protected kidney. To identify genes whose decreased expression potentially results in protection against glomerulosclerosis, the cDNAs expressed in −/− kidneys were subtracted from the cDNAs expressed in +/+ kidneys, resulting in a library (+1+) consisting of cDNAs down-regulated in the −/− protected kidney. cDNA clones were spotted on duplicate membranes and screened for differential expression using the −/− and +/+ subtraction libraries as probes. Differential expression of clones displaying differential hybridization to the −/− and +/+ library probes was confirmed by northern analysis of RNA from diabetic −/− and +/+ kidneys using the clones as probes. Nucleotide sequences were determined for each clone and compared by BLAST analysis to the sequences in the public domain databases.

We have obtained several partial cDNA clones whose altered expression is associated with the protection of the kidney from damage.

Induction of Diabetes with Streptozotocin

Two to three month old female mice were made diabetic (DB) with daily intraperitoneal injections of streptozotocin (STZ; 80-85 μg/g body weight, dissolved in 0.1 M citrate buffer at a concentration of 8-8.5 μg/ml) until blood glucose levels exceeded 200 mg/dL or for a maximum of 8 injections. Nondiabetic (ND) controls received daily injections of citrate buffer. After 4 or 10 weeks of diabetes (defined as maintenance of a blood glucose level of at least 300 mg/dL), the mice were sacrificed and the right kidney quickly removed, weighed, and frozen in liquid nitrogen or immediately processed for extraction of RNA. The left kidney was perfused through the dorsal aorta with phosphate buffered saline followed by 4% paraformaldehyde (PFA) in 0.15 NaCl, then removed, cut in half longitudinally and stored in 4% PFA for histological analysis.

RNA Isolation

Total RNA was isolated from kidneys of diabetic female GHR/BP +/+ and −/− mice after 4 or 10 weeks of diabetes induced by streptozotocin using the RNA STAT-60 Total RNA/mRNA Isolation Reagent according to the manufacturer's instructions (Tel-Test, Friendswood, Tex.).

cDNA Synthesis

Prior to cDNA synthesis, a portion (50 μg) of RNA was further purified to remove small RNAs using the RNeasy Mini protocol for RNA clean up as instructed by the manufacturer (Qiagen Inc., Santa Clarita, Calif.). cDNA then was synthesized using 1 μg of this purified sample of total RNA from one diabetic GHR/BP +/+ and one diabetic GHR/BP −/− mouse using the SMART PCR cDNA Synthesis Kit according to the manufacturer's instructions (CLONTECH, Palo Alto, Calif.).

Generation of cDNA Subtraction Libraries

Forward- and reverse-subtracted cDNA libraries were generated using the PCR-Select cDNA Subtraction Kit (CLONTECH, Palo Alto, Calif.) and the +/+ and −/− samples. One library (+/+ or G) included clones down-regulated in the diabetic −/− mice and the other library (−/− or H) included clones up-regulated in diabetic −/− mice.

Isolation of Individual Clones

After generating the cDNA subtraction libraries, the PCR product ends were made blunt by treatment with Pfu DNA polymerase (Stratagene, La Jolla, Calif.) and subcloned into a bacterial plasmid vector using the Zero Blunt TOPO PCR Cloning Kit as instructed by the manufacturer (Invitrogen Corp., Carlsbad, Calif.). Individual clones were obtained by plating on selective media.

Screening by Differential Hybridization

cDNA arrays of clones from the forward and reverse subtracted libraries were screened with probes made from each library using the PCR-Select Differential Screening Kit according to the manufacturer's instructions (CLONTECH, Palo Alto, Calif.).

Nucleotide Sequence Determination

Plasmid DNA from bacterial colonies carrying the differentially expressed cDNA inserts was isolated using the QIAprep Spin Miniprep Kit according to the manufacturer's instructions (Qiagen Inc., Santa Clarita, Calif.). Nucleotide sequences were determined by use of the ABI PRISM BigDye Terminator Cycle Sequencing Ready Reaction Kit with electrophoresis on the ABI PRISM 377 DNA Sequencer (PE Applied Biosystems, Foster City, Calif.).

Database Searches

Nucleotide sequences and predicted amino acid sequences were compared to public domain databases using the Blast 2.0 program (National Center for Biotechnology Information, National Institutes of Health). Nucleotide sequences were displayed using ABI prism Edit View 1.0.1 (PE Applied Biosystems, Foster City, Calif.).

Nucleotide database searches were conducted with the then current version of BLASTN 2.0.12, see Altschul, et al., “Gapped BLAST and PSI-BLAST: a new generation of protein database search programs”, Nucleic Acids Res., 25:3389-3402 (1997). Searches employed the default parameters, unless otherwise stated.

For blastN searches, the default was the blastN matrix (1,-3), with gap penalties of 5 for existence and 2 for extension.

Protein database searches were conducted with the then-current version of BLAST X, see Altschul et al. (1997), supra. Searches employed the default parameters, unless otherwise stated. The scoring matrix was BLOSUM62, with gap costs of 11 for existence and 1 for extension. The standard low complexity filter was used.

“ref” indicates that NCBI's RefSeq is the source database. The identifier that follows is a RefSeq accession number, not a GenBank accession number. “RefSeq sequences are derived from GenBank and provide non-redundant curated data representing our current knowledge of known genes. Some records include additional sequence information that was never submitted to an archival database but is available in the literature. A small number of sequences are provided through collaboration; the underlying primary sequence data is available in GenBank, but may not be available in any one GenBank record. RefSeq sequences are not submitted primary sequences. RefSeq records are owned by NCBI and therefore can be updated as needed to maintain current annotation or to incorporate additional sequence information.” See also http://www.ncbi.nlm.nih.gov/LocusLink/refseq.html

It will be appreciated by those in the art that the exact results of a database search will change from day to day, as new sequences are added. Also, if you query with a longer version of the original sequence, the results will change. The results given here were obtained at one time and no guarantee is made that the exact same hits would be obtained in a search on the filing date. However, if an alignment between a particular query sequence and a particular database sequence is discussed, that alignment should not change (if the parameters and sequences remain unchanged).

Northern Analysis

Positive clones, identified by the differential hybridization screen, were used as probes in Northern hybridization analyses to confirm their differential expression. Total RNA isolated from the kidneys of diabetic female +/+ and −/− mice was resolved by agarose gel electrophoresis through a 1% agarose, 1% formaldehyde denaturing gel, transferred to positively charged nylon membrane, hybridized in DIG Easy Hyb with the cDNA insert labeled by asymmetric PCR with Digoxigenin, and analyzed using anti-Digoxigenin-alkaline phosphatase conjugate and CDP-Star according to the manufacturer's instructions (Roche Diagnostics Corp., Indianapolis, Ind.).

Characterization of Clones

Clone G26

Insert size: 1527 bp query sequence (with gap) (SEQ ID NOs:1 and 2)

Nucleotide Database Search

Blast N:

Clone G26 is apparently a partial cDNA of a gene encoding a protein similar to Mus musculus kidney 3 beta-hydroxysteroid dehydogenase type 4 (Hsd3b4). The highest scoring DNA alignment (890 bits, E value 0.0) was of bases 3-560 of clone G26 with bases 1602-1048 of Hsd3b4. (Note that since the orientation of the cDNA inserts in the cloning vector was not known, “plus” was assigned arbitrarily for the purpose of the Blast alignment. So, since the match is to the minus strand of a known DNA, we assume that the strand labeled “plus” was actually the minus strand of G26.) The percentage identity was 96% ( 540/561), with 9 gaps.

There were four equally high scoring matches: Genbank BC013449.1, ref NM_{—}008294.1, dbj AB049424.1 and Genbank L16919.1. The first is an M. musculus cDNA “similar to hydroxysteroid dehydrogenase-4 delta <5>-3-beta”. The second is M. musculus hydroxysteroid dehydogenase-4, delta <5>-3-beta. The third is M. musculus clone:123-1, derived from 4× chromosome of T(X;4) 37H translocation. The fourth is the M. musculus cDNA (pK411A) 3-beta-hydroxysteroid dehydogenase/delta-5-delta-4 isomerase (Hsd3b4). The other high scoring DNA alignments were with genes closely related to Hsd3b4.

The highest human matches in the main database were

| | | |
|---|---|---|
| gi\|4504506\|ref\|NM_000862.1\| Homo sapiens | 76 | 7e−11 |
| hydroxy-delta-5-st . . . | | |
| gi\|9581790\|emb\|AL121995.12\|HSDJ920G3 Human DNA | 76 | 7e−11 |
| sequence fro . . . | | |
| gi\|257052\|gb\|S45679.1\|S45679 3 beta-hydroxysteroid | 76 | 7e−11 |
| dehydrog . . . | | |
| gi\|287843\|emb\|X53321.1\|HS3BHD54i H. sapiens | 76 | 7e−11 |
| mRNA for 3Beta-h . . . | | |
| gi\|23861\|emb\|X55997.1\|HS3B5H5E Human gene for | 76 | 7e−11 |
| 3-beta-5-hydr . . . | | |
| gi\|179467\|gb\|M38180.1\|HUMBHSD Human | 76 | 7e−11 |
| 3-beta-hydroxysteroid d . . . | | |
| gi\|177194\|gb\|M63397.1\|HUM3BHSD03 Human | 76 | 7e−11 |
| 3-beta-hydroxysteroi . . . | | |
| gi\|177190\|gb\|M35493.1\|HUM3BHSD Human placental | 76 | 7e−11 |
| 3-beta-hydro . . . | | |
| gi\|184398\|gb\|M27137.1\|HUMHSDB Human | 76 | 7e−11 |
| 3 beta-hydroxy-5-ene st . . . | | |
| gi\|184275\|gb\|M28392.1\|HUMHODB3 Human | 76 | 7e−11 |
| 3-beta-hydroxysteroid . . . | | |

However, in the human EST library, the top scores were

| | | |
|---|---|---|
| gi\|1578840\|gb\|AA071460.1\|AA071460 zm73f03.s1 Stratagene | 868 | 0.0 |
| neu . . . | | |
| gi\|1688820\|gb\|AA129037.1\|AA129037 zo11e12.s1 Stratagene | 819 | 0.0 |
| neu . . . | | |
| gi\|1578927\|gb\|AA071539.1\|AA071539 zm74b06.s1 | 791 | 0.0 |
| Stratagene neu . . . | | |
| gi\|1578624\|gb\|AA071072.1\|AA071072 zm73fo3.r1 Stratagene | 660 | 0.0 |
| neu . . . | | |

Thus, of the human sequences, Clone G26 is most similar to a human EST (gi:1578840), not to any of the human cDNAs reported in GenBank (see included alignments). This EST is conceivably the cognate human gene. However, there is a warning in the database to the effect that “there is evidence that suggests that the 384-well parent plate of this clone contains both human and mouse derived clones. Thus, the origin of this clone is uncertain.” The same warning appears for gi:1688820, gi:1578927, and gi:1578624.

Protein Database Search

Blast X: The best score in the main database was with Mus musculus-hydroxysteroid dehydrogenase-4, delta <5>-3-beta, ref NP_{—}03320.1 (score 187 bits, expect 2e-71, 95% identity).

In the human database, the best scores were for

| | | |
|---|---|---|
| gi\|4504507\|ref\|NP_000853.1\| (NM_000862) | 128 | 9e−48 |
| hydroxy-delta-5-ste . . . | | |
| gi\|179468\|gb\|AAA51831.1\| (M38180) | 128 | 9e−48 |
| 3-beta-hydroxysteroid deh . . . | | |
| gi\|4504509\|ref\|NP_000189.1\| (NM_000198) | 122 | 2e−46 |
| hydroxy-delta-5-ste . . . | | |
| gi\|12043436\|emb\|CAC19801.1\| (AL359553) dJ871G17.4 | 122 | 9e−45 |
| (novel 3- . . . | | |
| gi\|17488989\|ref\|XP_060821.1\| (XM_060821) | 122 | 9e−45 |
| similar to dJ871G1 . . . | | |
| gi\|17489003\|ref\|XP_060827.1\| (XM_060827) | 122 | 3e−44 |
| similar to 3 BETA- . . . | | |
| gi\|19979635\|gb\|AAM08704.1\| (AF252254) | 122 | 2e−39 |
| 3-beta-hydroxysteroid . . . | | |
| gi\|386780\|gb\|AAA36001.1\| (M28392) | 128 | 2e−39 |
| 3-beta-hydroxysteroid deh . . . | | |

Note that these proteins are functionally related.

Northern analysis: Down-regulated in protected (DB −/−) kidney
ND +/+>DB+/+=ND−/−>>DB−/−.

The characteristics of Hsd3b4 are discussed in Clarke, et al., Mol. Endocrinol., 7:1569-78 (1993). Its relationship to other types of beta-hydroxysteroid dehydrogenases is discussed in Payne, et al., J. Steroid Biochem. Mol. Biol., 53:111-8 (1995).

Clone H8

Insert size: 875 bp query sequence 875 bp (SEQ ID NO:2)

Nucleotide Database Search

Blast N: The best mouse match was to gp bF539644.1 EST 1197 bits e=0.0 97%. In the main database, the best match was to Genbank AC063194.8, H. Sapiens clone RP11-216N21, E value of 8e-40, score of 163 bits. Query 107-280 was aligned to subject 139697-139870, with 151/174 (86%) identities. There were several other alignments, too.

The score for the match to Homo sapiens ATPase, XM_{—}043092.1 was 40 bits, with E=0.008.

Looking at human ESTs, the top matches were

| | | |
|---|---|---|
| gi\|15755092\|gb\|BI763514.1\|BI763514 603050239F1 | 617 | e−174 |
| NIH_MGC_116 . . . | | |
| gi\|13293170\|gb\|BG399722.1\|BG399722 602441316F1 | 414 | e−113 |
| NIH_MGC_75 H . . . | | |
| gi\|5339253\|gb\|AI791537\|AI791537 oj41b11.y5 | 408 | e−111 |
| NCI_CGAP_Kid3 . . . | | |
| gi\|13340125\|gb\|BG433708.1\|BG433708 602497822F1 | 363 | 7e−98 |
| NIH_MCG_75 H . . . | | |
| gi\|12158598\|gb\|BF820247.1\|BF820247 | 359 | 1e−96 |
| CM0-RT0018-181100-701-c1 . . . | | |
| gi\|5054161\|gb\|AI733048.1\|AI733048 oj41b11.x5 | 333 | 6e−89 |
| NCI_CGAP_Kid3 . . . | | |
| gi\|15751872\|gb\|BI7660294.1\|BI760294 603045361F1 | 301 | 2e−79 |
| NIH_MGC_116 . . . | | |

Blast X: The top scoring sequences are:

| | | |
|---|---|---|
| gi\|18570892\|ref\|XP_088368.1\|Hypothetical protein | 379 | e−104 |
| XP_088368 | | |
| gi\|7304909\|ref\|NP_038505.1\| (NM_013477) ATPase, | 370 | e−102 |
| H+ transpor . . . | | |
| gi\|12643287\|sp\|P51863\|VA0D_MOUSE Vacuolar ATP | 370 | e−102 |
| synthase subu . . . | | |
| gi\|14776195\|ref\|XP_043091.1\| ATPase, H+ | 370 | e−102 |
| transporting, lysos . . . | | |
| gi\|15029719\|gp\|AAH11075.1\|AAH11075 (BC011075) | 367 | e−101 |
| Unknown (prot . . . | | |
| gi\|18543319\|ref\|NP_570080.1\| (NM_130724) CG2934 | 354 | 3e−97 |
| gene produc . . . | | |
| gi\|12585456\|sp\|Q25531\|WA0D_MANSE Vacuolar ATP | 352 | 4e−97 |
| synthase subu . . . | | |
| gi\|16259229\|gb\|AAK85455.2\|AC006611.2 (AC006611) | 337 | 2e−92 |
| Hypothetica . . . | | |
| gi\|17505761\|ref\|NP_491515.1\| (NM_059114) | 320 | 4e−87 |
| vaculolar ATPase [. . . | | |

XP_{—}088368.1 is a hypothetical protein; region 15.214 has been identified as a “ATP synthase (C/AC39) subunit. Sequence xP_{—}088368.1 was replaced on May 7, 2002 with gi:2048 5329, “similar to Ac39/physophilin (Homo sapiens)”

XP_{—}04309.1 is ATPase, H+ transporting, lysosomal (vacuolar protein pump), H. sapiens. It is also known as “similar to vacuolar ATP synthase subunit D”. Between query 3-818 and subject 9-279, there was 65% identity ( 179/272), with a score of 370 bits, E=e-102.

Best mouse match ref NP_{—}038505.1 370 bits e-102 65% vacuolar adenosine tri-phosphatase subunit D.

Northern analysis: Up-regulated in protected (DB−/−) kidney
DB −/− > >
ND −/− > =
ND +/+ =
DB +/+
   Clone H1

Insert size: 966 bp, query sequence 966 bp (SEQ ID NO:4)

Nucleotide Database Search

Blast N: Matches with GenBank U96635 Mus musculus ubiquitin protein ligase Nedd-4 mRNA (1780 bits, E 0.0), when bases 1-966 of H1 are aligned with bases 3775-4739 of the database sequence. The percentage identity is 98% ( 957/969), with 7 gaps.

There is an even higher (1804) scoring alignment with BC007184, M. musculus clone IMAGE:3591422.

The highest scoring human sequence is ref XM_{—}046129.1, Homo sapiens neural precursor cell expressed, developmentally down-regulated 4 (NEDD 4), with a score of 105 bits and E=4e-20.

There are identical scoring human ESTs, e.g., BG035335 and BF357965.

Protein Database Search

Blast X: Nothing significant. This is expected; the coding sequence of U96635 is at bases 612-2945, so the clone is in 3′ UTR of U96635.

Northern analysis: Up-regulated in protected (DB −/−) kidney
DB−/−>>DB+/+=14D+/+=ND−/−.

As described by Kumar, et al., Genomics, 40:435-446 (1997), Nedd4 encodes a protein which is strongly expressed during early CNS development. It contains a calcium/phospho lipid binding domain, three protein-protein interaction domains (the WW domains), and a ubiquitin-protein ligase domains. Ubiquitin labels proteins desired for degradation by substrate-specific ubiquitin conjugating enzymes and ubiquitin-protein ligases.

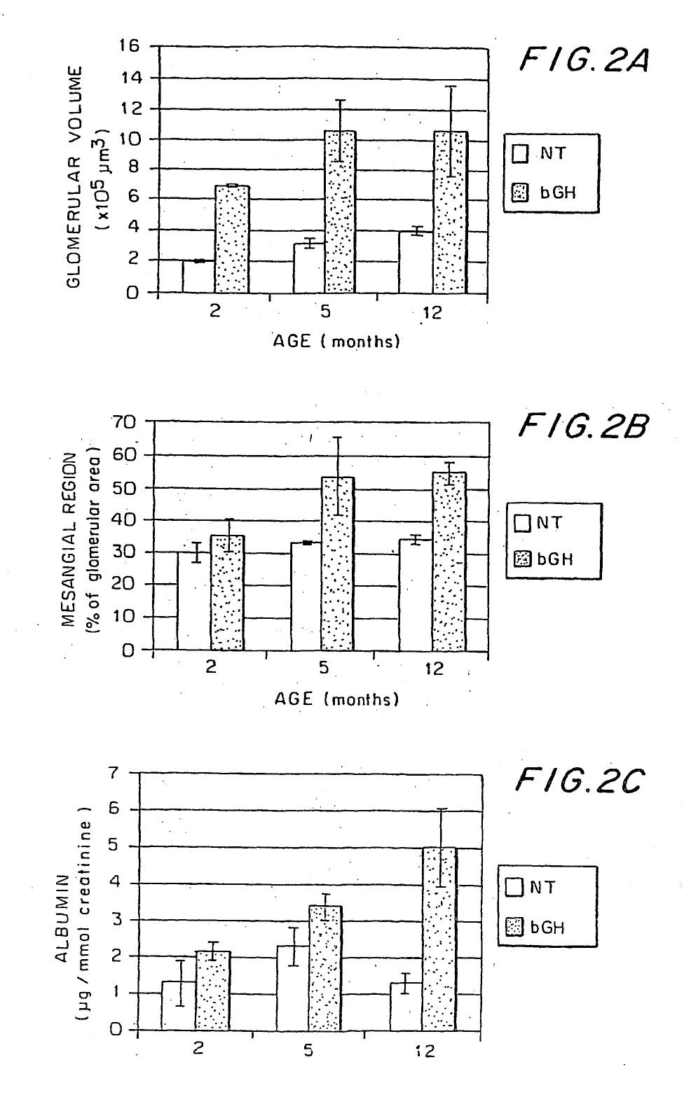

The alignment of mouse, human (Genbank D42055) and rat (Genbank U50842) DNA sequences is set forth in Kumar, FIG. 2.

Pirozzi, et al., J. Biol. Chem., 272:14611-6 (1987) has identified other human WW domain-containing proteins; these are aligned with, inter alia, Nedd-4 in FIG. 2.

Clone F1

Insert size: 1159 bp, query sequence 1159 bp (SEQ ID NO:5)

Nucleotide Database Search

Blast N: Matches with ref NM-019973.1 Mus musculus Son cell-proliferation protein mRNA (1931 bits, E=0.0, 99% identity between query 150-1159 and subject 413-1419), GenBank AF193607 Mus musculus truncated SON protein (Son) mRNA (1931 bits, E value=0.0, 99% identity between query 150-1159 and subject 413-1419) and with Genbank AF193597 Mus musculus SON protein mRNA (1931 bits, E value 0.0, 99% identity between query 150-1159 and subject 156-1162).

The best human match was to ref NM_{—}058183.1 Homo sapiens SON DNA binding protein mRNA, 922 bits, E=0.0, 86% identity of query 150-1159 to subject 414-1435.

Protein Database Search

Blast X: Matches with ref NP_{—}064357 and AAF23121.1 (AF193607) Mus musculus truncated SON protein (624 bits, E=e-178, 86% identity of query 3-1157 with subject 80-463) and GenBank AAF23120.1 (AF193606) Mus musculus Son protein (624 bits, E=e-178, 86% identity over 385 a.a.).

Please see also ref NP_{—}003094.2, the H. sapiens SON DNA-binding protein, KIAA1019 (score 497 bits, E=e-140, align query 3-1157 to subject 80-468 for 278/390 (71%) identities, 5/390 gaps).

Northern analysis: up-regulated in protected (DB −/−) kidney DB−/−>>ND−/−=ND+/+>>DB+/+.

The SON protein is discussed in Wynn, et al., “Organization and Conservation of the GART-SON-3SG locus in mouse and human genomes”, Genomics, 68(1):57-62 (2000); its gene is within the Down Syndrome Critical Region.

Of the human sequences, clone F1 is most similar to the SON DNA binding protein (gi:15055517) with 71% identity and e-140.

Clone G38

1050 bp Insert, 842 bp Query Sequence (SEQ ID NO:18)

Highest alignment score was with Genbank AF178454.1, Mus musculus anti-human apolipoprotein A monoclonal antibody 20 L Kappa light chain,

Identities= 538/565 (95%), score 888 bits, E=0.O.

Highest alignment score with a Human mRNA was with Genbank S65921.1, anti-colorectal carcinoma light chain glycoprotein CANAG-50 specific IgG1 Kappa

Identities= 358/387 (92%), score 529, e-148

At the protein level, the highest score was with gi 1310829, pdb 1KNO, Igg2a-p-nitrophenyl methyl phosphonate. The highest human score was with gb AAB28160.1 (S65921) an anti-colorectal carcinoma antibody, score 258, E=3e-71. See Zemel, et al., Molec. Immunol., 31: 127-37 (1994).

Northern analysis: up-regulated in protected (DB−/−) kidney.

In like manner, we have identified several additional clones, with the following Northern analyses (ND nondiabetic/no STZ; DB diabetic/STL; NT nontransgenic; HO transgenic for GHR/BP Knock out):

G9: NDNT < DBNT = DBHO = NDHO

(SEQ ID NO:29)

Cytochrome oxidase III
G16: NDNT = DBNT < DBHO < NDHO

(SEQ ID NO:30)

Hypothetical protein
G24: DBNT > NDNT = DBHO = NDHO

(SEQ ID NO:31)

Homolog to ERG2 protein
G28: NDNT = DBNT > DBHO = NDHO

(SEQ ID NO:32)

Glutathione peroxidase 3

### Example 2

Differential Expression of L Genes in Kidneys of Nontransgenic Mice as a Result of Streptozotocin Treatment

End stage renal disease (ESRD) is a costly outcome of diabetes, yet it is a difficult complication to prevent. Factors that influence its development include heredity, blood glucose levels, and blood pressure. ESRD is a progressive disease that can take many years to develop. It can sometimes be treated, and even reversed, if caught at an early stage. Thus, an understanding of the molecular basis for the development and progression of diabetic nephropathy culminating at ESRD is crucial for the diagnosis, treatment and prevention of this debilitating disease.

Streptozotocin-induced diabetes in mice provides an excellent model system for studying the development of insulin-dependent diabetes mellitus (IDDM) nephropathy, at both the physiological and molecular level. We, and others, have used this model system to examine the role of specific genes or gene products in the development of diabetic nephropathy leading to glomerulosclerosis. While significant advances have been made, much information is still lacking. In an attempt to further our knowledge of the molecular mechanisms resulting in diabetic nephropathy, we are using a polymerase chain reaction (PCR)-based cDNA subtraction method to compare populations of mRNAs expressed in kidneys of streptozotocin-induced diabetic mice at various developmental stages of the disease with populations of mRNAs expressed in healthy kidneys by creating libraries containing virtually all of the gene products (cDNAs; novel and known) involved in the progression of glomerulosclerosis. Nephropathy-correlated genes, thus identified, may be used as molecular markers for disease progression and as therapeutic drugs or drug targets.

In this example, subtractive hybridization was used to identify genes differentially expressed in diabetic (STZ-treated) and control mice. These mice, unlike those of Example 1, were all nontransgenic.

Several partial cDNA clones were identified whose altered expression is associated with kidney damage. The protocol was the same as for Example 1, except as set forth below.

RNA isolation

Total RNA was isolated from kidneys of wildtype female mice after 4 or 10 weeks of diabetes induced by streptozotocin, as well as similarly aged non-diabetic wildtype controls, using the RNA STAT-60 Total RNA/mRNA Isolation Reagent according to the manufacturer's instructions (Tel-Test, Friendswood, Tex.).

cDNA Subtraction Libraries

One library (E) included clones up-regulated in diabetic mice and the other library (F) included clones down-regulated in diabetic mice.

Northern Analysis

Positive clones, identified by the differential hybridization screen, were used as probes in Northern hybridization analyses to confirm their differential expression. Total RNA isolated from the kidneys of female mice after 10 weeks of diabetes induced by streptozotocin, as well as non-diabetic controls, was resolved by agarose gel electrophoresis through a 1% agarose, 1% formaldehyde denaturing gel, transferred to positively charged nylon membrane, hybridized in DIG Easy Hyb with the cDNA insert labeled by asymmetric PCR with Digoxigenin, and analyzed using anti-Digoxigenin-alkaline phosphatase conjugate and CDP-Star according to the manufacturer's instructions (Roche Diagnostics Corp., Indianapolis, Ind.).

Characterization of Clones

Clone F1

The results of a sequence search for this clone are discussed in Example 1. Besides being up-regulated in protected (DB −/−) kidney, it was down-regulated in diabetic +/+ kidney
ND+/+>>DB+/+
   Clone F4

Insert size: 616 bp query sequence 616 bp (SEQ ID NO:6)

Nucleotide Database Search

Blast N: Matches with ref. NM_{—}010594.1 Mus musculus kidney androgen regulated protein (Kap) mRNA
E Value=0.0 (1154-bits) (99% identity of query 9-612 with subject 3-606)

With H. sapiens FKSG22 mRNA (AF319957.1), the alignment yielded a score of 1098 bits (E=0.0) for query 33-612 with subject 1-580 ( 576/581 [99%] identities).

Protein Database Search

Blast X: Matches with ref. NP_{—}034724.1 (NM 010594) Mus musculus kidney androgen regulated protein and human AAH08576.1 kidney androgen regulated protein.

E value=1e-61 (235 bits) (99% identity of query 54-416 with subject 1-121)
Northern analysis: Down-regulated in diabetic kidney
ND+/+>>>DB+/+
   Of the human sequence, clone F4 (and F6) is most similar to FKSG22 mRNA (gi:12276125), implicated in ovarian cancer, and kidney androgen regulated protein mRNA (gi:14250295); e=0. Both mRNAs encode the protein, KAP.

The kidney androgen-regulated protein (KAP) is encoded by a relatively abundant mRNA (4% of poly(A) RNA in male mouse kidney). The concentration of KAP mRNA is 3-5 fold higher in males, or in testosterone-treated females, than in untreated females. It appears to be expressed in the epithelial cells of the proximal convoluted tubules, in particular by cells of the S1, S2 and S3 segments. Several possible hormone-responsive elements have been identified in the 5′-flanking region. The KAP gene is a single copy gene, but seems to be highly polymorphic. See generally Niu, et al., DNA & Cell Biol., 10:41-8 (1991).

For KAP, see also Homo sapiens, kidney androgen regulated protein, clone MCG:18182, IMAGE:4155347, mRNA, complete cds. Accession #BC008576, version #BC008576.1, GI: 14250295.

Clone F5 (SEQ ID NO:7)

Insert size: −1000 bp, query sequence 881 bp, SEQ ID NO:6

Nucleotide Database Search

Blast N: Top matches are with (1) gi20881938, ref XM_{—}127876.1 “Mus musculus similar to data source: SPTR, source key:Q9Z0S6, evidence:ISS˜putative-similar to CLAUDIN-10” (score 1233 bits E 0.0), (2) gi 21617848, ref NM_{—}023878.1, “Mus musculus claudin 10 (Cldn10) (score 125 bits E 0.0)”, and (3) gi12860620, dbj AK020131 “Mus musculus 12 days embryo male wolffian duct includes surrounding region cDNA; RIKEN full-length enriched library”, (1215 bits E 0.0), and several other claudin-10-related sequences (gb BC021770.1, dbj AK055855.1, BC029019.1, ref NM_{—}021386.1, gb AF124425.1).

The highest-ranked human sequence was gi 16550687, dbj AK055855.1, “Homo sapiens cDNA FLJ31293 fis, clone KIDNE2007569, moderately similar to claudin 10.” See also gi 16444694, emb AL357061.19, and gi 17475396, ref XM 007076.4.

The cognate human claudin-10 sequence is ref. NM_{—}006984.1 (165 bits, E=1e-37) or Genbankc U89916. See also gi 17475396, ref XM 007076.4, and gi15012044, Genbank BC010920.

Protein Database Search

Blast X: Best matches were with (1) gi 20881939, ref xP_{—}127876.1, “similar to data source:SPTR, source key: Q9Z0S6, evidence:ISS˜putative-similar to CLAUDIN-10 [Mus musculus]” (236 bits, E=1e-74); (2) gi 21617849, ref NP_{—}076367.1, “claudin 10” (234 bits E=3e-74), (3) gi 16550688, dbj BAB71030.1, AK055855, “unnamed protein product [Homo sapiens]” (225 bits, E=4e-71); and (4) gi 18256029, gb AAH21770.1, “similar to RIKEN cDNA 672045616 gene [Mus mussculus]” (236 bits, E=1e-68).

The cognate human protein sequence is ref NP_{—}008915.1 (E=6e-40).

Northern analysis: Down-regulated in diabetic kidney
ND+/+>DB+/+

Claudins are integral membrane proteins localized as tight junctions, see Kubota, et al., Curr. Biol. 9:1035-8 (1999); Monta, et al., J. Cell. Biol., 145:579-88 (1999); Monta, et al., Proc. Nat. Acad. Sci. (USA), 96:511-16 (1999).

Clone F6 (SEQ ID NO:8)

This is a slightly shorter version (611 bp) of clone F4.

Clone E39

Insert size 981 bp (SEQ ID NOs:15 and 16)
Blast N Similar to gb U65535.1 mouse immunoglobulin kappa-chain mRNA, 346 bits. 2e-92

The best human match was to gbS65921.1, anti-colorectal carcinoma light chain=glycoprotein CANAG-50 specific IgG1 kappa 338 bits, 4e-90. See Schable, et al., Eur. J. Immunol., 29:2082-6 (1999).

Blast X

Human protein cognate: 1FH5L, pdb 1FH5 Chain L crystal structure of the Fab Fragment of the Monoclonal Antibody Mak 33, 93 bits 5e-19.

See also Mouse gb AAC04542.1, monoclonal antibody kappa light chain [mus musculus] 61 bits, 8e-16.
Northern analysis: Up-regulated in diabetic kidney (DB+/+>>ND+/+) and damaged bGH kidney (bGH>NT).
   Clone F27
   −1000 bp insert 820 bp query sequence (SEQ ID NO:17)
   End 337 n; similar to ref NM 013559.1 mus musculus heat shock protein, 105 kDa (Hsp 105)
   Blast N mRNA, 3387 nt
   Identities= 329/338 (97%), E=e-173, 607 bits
   ref XM_{—}036358.1 Homo sapiens heat shock 105 kD (HSP105B) mRNA, 220 bits, 7e-55, 83%.
   dbj BAA11035.1 (D67016) heat shock protein 105 KD alpha [mus musculus], 182 bits, 3e-45, 75%.
   Human Protein Cognate:
   Blast X. Heat shock 105 kD. [homo sapiens] ref XP-036357.1
   Identities= 95/136 (69%), 1e-42
   Rest of sequence is novel

Northern analysis: Down-regulated in diabetic kidney (ND+/+>DB+/+)

The top matches in the nucleic acid alignment were

| | | |
|---|---|---|
| gi\|7305168\|ref\|NM_013559.1\| Mus musculus | 607 | e−173 |
| heat shock protein . . . | | |
| gi\|840651\|gb\|L40406.1\|MUSHSPE Mus musculus | 607 | e−173 |
| heat shock prote . . . | | |
| gi\|1001008\|dbj\|D67016.1\|D67016 Mus wagneri | 607 | e−173 |
| mRNA for heat sh . . . | | |
| gi\|1001010\|dbj\|d67017.1\|d67017 Mus wagneri | 599 | e−171 |
| mRNA for heat sh . . . | | |

For the top match, NM_{—}013559, Mus musculus heat shock protein 105 kDa, query 1-337 aligned to subject 894-1231 with 329/338 (97%) identities and 1 gap.

The best NA alignment to a human sequence was of query 1-328 to XM_{—}036358.1, Homo sapiens heat shock 105 kD, subject 797-1125, with 274/329 identities (83%) and 1 gap.

For the amino acid sequence corresponding to NM_{—}013559.1, that is, the Mus musculus 105 kDa heat shock protein, See Morozov, et al., FEBS Lett., 371(3):214-18 (1995), and has the ID of NP_{—}038587.1 (which refers to it as a “110 kDa” protein).

In like manner, additional clones have been identified, with the following Northern analyses (ND, nondiabetic; DB, diabetic):
F2: ND>DB; Far upstream element (FUSE) binding protein 1 (SEQ ID NO:23)
F16: ND>DB; Unknown (SEQ ID NO:34)
F21: ND<DB; Unknown (SEQ ID NOS:24, 25)
F38: ND>DB; Palmitylated serine/threonine kinase (SEQ ID NO:26)
F39: ND>DB; Phosphotriesterase related protein (SEQ ID NO:27)
F40: ND<DB; Similar to mouse testicular tumor differentially expressed gene (TDE) 1 (SEQ ID NO:28)

FUSE binding protein 1 is discussed in Duncan, et al., Genes & Development, 8:465-80 (1994). The c-myc proto-oncogene stimulated by it is involved in normal cell proliferation and programmed cell death. The FUSE is required for maximal transcription of c-myc. FBP stimulates expression of c-myc in a FUSE-dependent manner and is believed to be involved in regulation of c-myc.

A 305-aa embryo-derived serine/threonine kinase (EDPK), which we have identified with clone F38, was isolated by Kurioka, et al., Biochim. Biophys. Acta, 1443: 275-84 (1998). It is believed to play a role in intracellular signaling during embryogenesis. Its catalytic domain is 271 a.a. Another serine/threonine kinase, Krct, is described in Stairs, et al., Human Molec. Genet., 7: 2157-66 (1998).

A mouse cDNA (mpr56-1) with homology to a prokaryotic parathion hydrolase (phosphotriesterase)-encoding gene is discussed in Hou, et al., Gene, 168: 157-63 (1996). This gene is expressed primarily in liver, and post-natally. It is underexpressed in cystic kidneys. Expression is decreased upon acute renal injury induced by a single intraperitoneal injection of folic acid. The protein is 349 a.a. There is greater than 50% a.a. similarity to the procaryotic prathion hydrolase.

The human TDE homologue is analyzed in Bossolasco, et al., Molec. Carcinogenesis, 26: 189-200 (1999). It is 7890 homologous to the mouse TDE amino acid sequence.

### Example 3

Differential Expression of Genes in Kidneys of Mice as a Result of Overproduction of Growth Hormone

In this example, genes were identified which were differentially expressed in (1) transgenic mice with kidney damage as a result of overexpression of bovine growth hormone, as compared to (2) nontransgenic control mice.

A PCR based cDNA subtraction strategy was used to create libraries containing genes potentially involved in the progressive development of nephropathy. Total RNA was isolated from the kidneys of bGH and nontransgenic (NT) control mice at 2 months, 5 months, and 12 months of age and used to create the two cDNA libraries for each time point. The bGH libraries at each time point consisted of cDNAs upregulated in the bGH as compared to NT mice. The NT libraries consisted of cDNAs down-regulated in the bGH as compared to NT mice (i.e., upregulated in the NT as compared to the bGH mice). Subsequently, cDNA clones from each library were spotted on replicate membranes. Differential hybridization of the two membranes, one probed with the bGH library and the other probed with the NT library, was used to select differentially expressed clones. Differential hybridization of clones from the two libraries at 2 months of age showed no differential expression. At 5 months of age a number of differentially expressed clones were observed. At 12 months of age an even larger number of differentially expressed clones were seen. Thus far, Northern Analysis has revealed that several clones from the 12 month bGH library are highly expressed in the kidneys of bGH mice as compared to NT controls. Furthermore, their expression increases with time, correlating with development of glomerulosclerosis.

The protocols were the same as for Example 1, with the following exceptions.

Mice

The mice had a B6/SJL genetic background. Inserted in their genome was the bGH cDNA clone whose expression was driven by the mouse metallothionein I transcriptional regulatory sequences.

RNA Isolation

Total RNA was isolated from kidneys of 2, 5 and 12 month old female bovine growth hormone transgenic (bGH) mice and non-transgenic controls using the RNA STAT-60 Total RNA/mRNA Isolation Reagent according to the manufacturer's instructions (Tel-Test, Friendswood, Tex.).

cDNA Subtraction Libraries

The forward (bGH) subtracted library included clones up-regulated in bGH mice and the reverse (NT) library included clones down regulated in bGH mice.

Screening by Differential Hybridization

cDNA arrays of clones from the forward and reverse subtracted libraries at each time point (2, 5, and 12 months of age) were screened with probes made from each library of the corresponding time point using the PCR-Select Differential Screening Kit according to the manufacturer's instructions (CLONTECH, Palo Alto, Calif.).

Northern Analysis

Positive clones, identified by the differential hybridization screen, were used as probes in Northern hybridization analyses to confirm their differential expression. Total RNA isolated from the kidneys of 2, 5 and 12 months of age female bGH and non-transgenic mice was resolved by agarose gel electrophoresis through a 1% agarose, 1% formaldehyde denaturing gel, transferred to positively charged nylon membrane, hybridized in DIG Easy Hyb with the cDNA insert labeled by asymmetric PCR with Digoxigenin, and analyzed using anti-Digoxigenin-alkaline phosphatase conjugate and CDP-Star according to the manufacturer's instructions (Roche Diagnostics Corp., Indianapolis, Ind.).

Characterization of Clones

Genes up regulated in the 2, 5 or 12-month bGH libraries are considered unfavorable, and those up-regulated in the corresponding NT libraries are considered favorable, i.e., likely to encode a protective protein.

Clone AS (12 month bGH library)

Insert 334 bp, query sequence 334 bp (SEQ ID NO:9)

By BlastN, the highest similarity was to M21796.1, Mouse (M. spretus) Ig germline Kappa chain gene C-region, exon, with a score of 640 bits (E=0.0), and 99% identity of query 8-334 with subject 1287-1613. Highest similarity to human clone was S65921.1 anticolorectal carcinoma light chain=glycoprotein CANAG-50 specific IgG1 kappa (579 bits, 97% identity) e-163.

By BlastX, there were a very large number of sequences with the same score (86 bits) and E value (9e-17). Almost all were immunoglobulin genes. The best matching human clone was AAB28160.1 (S65921) anti-colorectal carcinoma light chain (86 bits, 97% identity 9e-17).

Northern analysis: Expression in kidneys of bGH-transgenic mice increases with age and is greater than levels seen in age-matched nontransgenic control mice. Highly expressed in spleen, lung, intestine, stomach, kidney, and omental fat; very low levels in liver, heart, gastrocnemius, and brain of nontransgenic mouse.

Clone A34 (12 Month bGH Library)

Insert size: 503 bp, query 503 bp (SEQ ID NO:10)

By BlastN, this has a score of 636 bits (E=0.0) for a 99% identical alignment of query 139-463 to subject 1323-999, the subject being emb X03690.1, M. musculus mRNA for Ig heavy chain constant region mu(b) allele. The best human match was to AF144029.1 Homo sapiens MDM2 gene, intron 9 and exon 10, partial sequence (208 bits, 2e-51, 99% identity).

By blastX, the score is 224 bits (E=3e-58) for a 99% identical alignment to pir A24976, Ig mu Chain C region (allele b) of the mouse. The best human alignment was to AAH01872.1 Unknown (191 bits, 4e-49), 84% identity) as well as many others.

Northern analysis: Expression in kidneys of bGH-transgenic mice increases with age and is greater than levels seen in age-matched nontransgenic control mice.

Clone A39 (12 Month bGH Library)

Insert Size: 482 bp, query sequence 482 bp (SEQ ID NO:11)

By BlastN the highest score (879 bits, E=0.0) was with BC010327.1, Mus musculus, clone MGC:6533, image:2651776, 435/457 identity (99%) of query 26-482 with subject 425-881. Next best (782 bits) was a mouse mRNA for gamma-2b immunoglobulin (emb V00799.1). The best human match was S65761, anti-colorectal carcinoma heavy chain glycoprotein CANAG-50 specific IgG1 kappa antibody. The score was 300 bits (E=5e-79), with 212/233 (90%) identity aligning query 133-365 to subject 513-745.

By BlastX BC010327 was again the highest scorer (233 bits, E=4e-61), with 117/154 (75% identity) in query 19-480 vs. subject 122-275. The next best score was to AAA 51630.1 Ig H-chain (v-region from MPC11) of mus musculus (226 bits, 8e-59, 75% identity). And the highest human scorer was AAB28159.1, corresponding to S65761. The observed identity was 87/157 (55%).

For information on gamma 2b transgenic mice, see Storb, et al., Immunol. Res., 13:291-8 (1994), and Ann. NY Acad. Sci. 546:51-6 (1988).

Northern analysis: Expression in kidneys of bGH-transgenic mice increases with age and is greater than levels seen in age-matched nontransgenic control mice.

Clone A48 (12 Month bGH Library)

Insert 430 bp, query sequence 430 bp (SEQ ID NO:12)

By BlastN, several sequences produced substantially equally significant alignment (E value 0.0):

BC008237.1 Mus musculus, similar to immunoglobulin heavy chain 4 (serum IgG1), clone MGC:6522 IMAGE:Z651217 mRNA

BC002121.1 mus musculus similar to immunoglobulin heavy chain 4 (serum IgG1), clone mGC:6628 IMAGE:3491766, mRNA

L35252.1 mouse germ line immunoglobulin gamma-1 (Ighb) mRNA

Best human match was S65761.1 anticolorectal carcinoma heavy-chain=glycoprotein CANAG-50 specific IgG1 kappa antibody (684 bits, e=0.0, 97% identity).

Note that these were all Ig-related.

By BlastX, the best score was for Genbank AAB59665 (M60434), score 254, E-1e-67. This is an Ig heavy chain constant region from Mus domesticus. 116/122 95% identity of query 3-368 to subject 227-348. The best human alignment was to S65761 anti-colorectal carcinoma (see above) (204 bits, 3e-53, 94% identity).

Northern analysis: Expression in kidneys of bGH-transgenic mice increases with age and is greater than levels seen in age-matched nontransgenic control mice.

Clone B45 (12 Month NT Library)

Insert Size: 610 bp, query sequence 610 bp (SEQ ID NO:13)

By BlastN, the highest scoring similarity was with ref NM_{—}010594.1, Mus musculus kidney androgen regulated protein mRNA (1170 bits, E=0.0), for 99% identity of query 9-610 with subject 4-605. Note also the high score for the closely related sequence M22810.1 (1168 bits). Best human match was to AF319957 Homo sapiens FKSG22 mRNA (1132 bits, E=0.0, 99% identity).

By BlastX, the highest score was for ref NP_{—}034724.1 and (p15267) kidney androgen regulated protein (238 bits, E of 3e-62), for 100% identity of query 53-415 with subject 1-121. The rat homologue, ref NP_{—}434689.1, scored 103 bits, E=1e-21. PAG50272.1 corresponds to the best human BLASTN match, Homo sapiens FKSG22 mRNA.

Northern analysis: Expression in kidneys of bGH-transgenic mice is less than levels see in age-matched nontransgenic control mice and decreases with age.

Clone C22 (5 month bGH library)

query sequence 731 bp (SEQ ID NO:14)
By Blastn, the best alignment is to Genbank BC016887.1 Mus musculus clone MGC:18401 IMAGE:4242174 mRNA and Genbank AF260580.1, Mus musculus disabled-2 p96 (Dab2) gene, score of 1243 bits (E 0.0), for 97% alignment of query 1-729 to subject 28489-27769. The best human alignment (58 bits) is to the human homologue, Genbank AF205890.1 (AFX 218839S2), and AC008846.7, E=7e-06, 43/47 identities.

No significant Blast X results, probably in 3′ noncoding region.

Either this human sequence, or the much longer mouse sequence, could be used as a probe to isolate the full length human DNA.

Dab2 is a widely expressed relative of Dab1, a neuron-specific signal transduction protein that binds to and receives signals from members of the low-density lipoprotein receptor family. Dab2 contains a phosphotyrosine-binding domain which binds peptides containing the sequence FXN-PXY. Dab2 may therefore be both a signal transduction protein and an adaptor protein that regulates protein trafficking. See Morris, “Disabled-2 colocalizes with the CDLR in clathrin-coated pits and interacts with AP-2”, Traffic, 2(2):111-23 (2001). It also may be a transcriptional activator, see Cho, et al., Biochem. J., 352:645-50 (2000).

This clone was identified as being up-regulated in kidneys of 5 month old bGH mice as compared to age-matched nontransgenic control mice, but this differential expression has not been confirmed by Northern analysis.

In like manner, additional clones have been identified, with Northern analyses (NT, nontransgenic; bGH, transgenic for bGH) given below:
B3: NT>bGH; Unknown (SEQ ID NOs:19, 20)
B46: NT>bGH; NAS˜hypothetical protein˜putative (SEQ ID NO:33)

### Example 201

Use of Somatic Gene Therapy with KAP mRNA to Protect Against Diabetic Nephropathy.

Specific Aims

Kidney damage is a frequent complication of both type I and type II diabetes, often ending in kidney failure, or end-stage renal disease (ESRD). Diabetic nephropathy, the single most common cause of ESRD, is a progressive disease that takes several years to develop and often goes undiagnosed. Our long-term goal is to design specific, targeted markers and therapeutic approaches for the diagnosis, treatment, and prevention of human diabetic kidney disease. Toward this end, we have identified a gene, encoding kidney androgen-regulated protein (KAP), whose expression decreases markedly with increasing kidney damage, in both a diabetes-dependent and a diabetes-independent mouse model of glomerulosclerosis. We believe that this gene, through manipulation of its expression, could provide a means for protecting the kidney from damage. This has tremendous therapeutic potential since KAP is a relatively small peptide, is possibly secreted, and appears to have a human homologue. Based on our findings, we propose the following hypothesis and specific aims.

Hypothesis: Maintenance of high levels of KAP expression will protect the kidney from glomerular hypertrophy and diabetic damage. We have observed high levels of KAP mRNA isolated from kidneys of nondiabetic female mice. In comparison, KAP mRNA levels are dramatically decreased in kidneys of streptozotocin (STZ)-induced diabetic mice. Furthermore, KAP mRNA levels progressively decline with age in nondiabetic bovine growth hormone (bGH)-transgenic mice. These mice exhibit increasingly severe glomerular hypertrophy and eventually glomeruloscierosis, hallmarks of the early stages of human diabetic nephropathy. Since low levels of KAP mRNA are associated with kidney damage, we hypothesize that maintenance of high levels of KAO expression will protect the kidney from damage. We will test this hypothesis by expressing KAP in the kidney using a heterologous promoter and then assessing kidney damage after induction of diabetes.

Specific Aim 1: To constitutively express KAP in the mouse kidney. KAP expression is normally restricted to proximal renal tubule cells and is under the control of several different hormones. In order to obtain constitutive expression of KAP, the full-length mouse cDNA encoding KAP will be placed under the direction of the kidney specific mouse gamma-glutamyl transpeptidase (GGT) type II promoter and transferred into mouse embryos by pronuclear microinjection. Individual lines will be established from several founding animals expressing the KAP transgene. In anticipation that expression from the kidney-specific promoter might be low, additional mouse lines will be generated with the KAP cDNA placed under the direction of the stronger mouse metallothionein-I (MT) promoter that expresses in several other tissues in addition to the proximal tubules.

Specific Aim 2: To induce diabetes in the KAP-transgenic mice and assess the degree of kidney damage. To test whether KAP expression can prevent diabetic nephropathy, diabetes will be induced in KAP-transgenic mice as well as nontransgenic control mice by injection of streptozotocin. Glomerular hypertrophy, mesangial cell expansion and urinary albumin excretion (UAE) will be assessed following a period of hyperglycemia. Observance or kidney measurements for the diabetic KAP-transgenic mice that more closely resemble those of nondiabetic rather than diabetic nontransgenic controls will support the hypothesis that maintenance of KAP-expression will protect the kidney from diabetic damage.

Characterization of the Kidney Androgen-Regulated Protein Gene

Expression of the HAP gene in mouse kidney is under the complex control of several hormones, including androgen, estrogen, thyroid hormone and the growth hormone/insulin-like growth factor-I (GH/IGF-I) axis (Cebrian et al., 2001; Meseguer and Catterall, 1990; Meseguer and Catterall, 1992). Expression is confined almost exclusively to epithelial cells of the renal proximal tubule (Meseguer and Catterall, 1987). Expression in the cortical S1 and S2 segments is androgen-dependent while expression in the medullar S3 segment is androgen-independent (Meseguer and Catterall, 1990; Meseguer et al., 1989). Expression of KAP also occurs in the mouse uterus, but only during the perinatal period (Cebrian et al., 2001; Kasik and Rice, 1993).

The KAP gene contains four exons and three intervening sequences that span 3807 nucleotides of the mouse genome (Niu et al., 1991). Its mRNA is 611 nucleotides in length (Niu et al., 1991). KAP gene regulation occurs primarily at the level of transcription with protein expression closely paralleling mRNA expression. The 121 amino acid protein product of KAP migrates with an apparent molecular mass of 20 kDa (Cebrian et al., 2001; Meseguer et al., 1989). A hydrophobic N-terminal domain forms a putative 18 amino acid signal peptide (Meseguer et al., 1989). The remainder of the protein is highly negatively charged and contains amino acid clusters similar to those associated with proteins with short half-lives (Meseguer et al., 1989). Immunohistochemical assays of mouse kidney sections identified the protein in the same tissues and at the same relative levels as the mRNA (Cebrian et al., 2001). Although the function of KAP remains elusive, it has recently been determined that the protein specifically interacts with cyclophilin B and that overexpression of KAP in stably transfected proximal tubule cells significantly decreases the toxic effects of cyclosporine A (Cebrian et al., 2001).

Importance

Up to 15.7 million Americans, or approximately 5.9% of the population, are thought to have diabetes. While two-thirds have been diagnosed, one third are not aware that they have the disease. Many people first become aware that they have diabetes when they develop one of its complications, such as kidney disease, heart disease, nerve disease or blindness. Ten to twenty-one percent of all people with diabetes eventually develop kidney disease. Diabetic nephropathy is the most common cause of ESRD, a condition that requires dialysis or a kidney transplant for survival. Approximately 40% of the new cases of ESRD are attributable to diabetes, and this incidence is increasing more rapidly than any other cause.

Amino acid sequence analysis suggests that KAP may be a small, secreted protein (Meseguer et al., 1989). In addition, a human cDNA has been identified that is 99% identical to the mouse cDNA at the nucleotide level and 100% identical at the amino acid level. If shown to have protective abilities, KAP could be utilized as a therapeutic drug for the prevention of diabetic nephropathy in humans.

Preliminary Studies

We have utilized two different mouse models that develop kidney damage by presumably independent means, the streptozotocin (STZ)-induced type I diabetic mouse and the MT/bGH-transgenic mouse. A PCR-based method of subtractive hybridization was used to create libraries that contained cDNAs either up-regulated or down-regulated in the damaged kidney in comparison to the undamaged control kidney. A cDNA encoding KAP was identified in the libraries of down-regulated cDNAs from both mouse models of kidney damage. Data describing the assessment of kidney damage and showing the down-regulation of the KAP gene in the two mouse models are presented below.

Analysis of Kidney Damage in Streptozotocin-Induced Diabetic Mice

Diabetes was induced by multiple injection of STZ as described by Bellush et al. (2000). In short, 2-month-old female 129 Ola/BalbC mice received daily injections of 85 mg STZ/kg body weight until their blood glucose levels were at ease 250 mg/dl. Control mice received a similar number of injections of citrate buffer without the STZ. Within one week, most of the STZ-injected (DB) mice had blood glucose levels of 300-500 mg/dl that were maintained throughout the duration of the study. The blood glucose levels of nondiabetic (ND) control mice remained below 100 mg/dl. Mice were sacrificed 4 or 10 weeks after the onset of diabetes. Several parameters reflecting the extent of kidney damage were assessed. Glomerular hypertrophy was observed in the DB mice in comparison with the ND mice at both the 4 and 10 wk timepoints. (FIG. 1A) A significant elevation of urinary albumin excretion (UAE) was observed only at the 10 wk timepoint. (FIG. 1B) Both changes are indicative of kidney damage. Measurements of mesangial cell proliferation, another indicator of damage, are in progress.

Decreased KAP mRNA Expression in Diabetic Kidneys

In an effort to identify genes involved in the progression of diabetic nephropathy, we performed a PCR-based method of subtractive hybridization using RNA extracted from kidneys of streptozotocin-induced (type I) diabetic female mice and nondiabetic controls collected 10 weeks after the establishment of diabetes. Two cDNA subtraction libraries were created, one (DB) containing cDNAs up-regulated in the diabetic kidney in comparison to the nondiabetic kidney, and one (ND) containing cDNAs down-regulated in the diabetic kidney. cDNAs exhibiting high sequence similarity to the KAP nucleotide sequence were isolated multiple times from the library of down-regulated cDNAs. Northern analysis confirmed the decreased expression in the diabetic kidney.

Analysis of Kidney Damage in MT/bGH-Transgenic Mice

Expression of the bovine growth hormone (bGH) cDNA under the direction of the metallothionein-I promoter (MT) results in a giant mouse that is approximately 1.5-9 times the size of nontransgenic mice (Chen et al., 1990; Quaife et al., 1989). These MT/bGH mice develop severe kidney damage and die prematurely (Quaife et al., 1989). We have assessed several parameters reflecting the extent of kidney damage in these mice as a function of age. Significant increases in glomerular volume (as determined by Analysis of Variance (ANOVA); P<0.05) were observed in the MT/bGH mice in comparison with the nontransgenic. (NT) mice at the 2 and 5 month timepoints but not at 12 months (FIG. 2A). A significant increase in mesangial cell area was observed only at the 12 month timepoint (FIG. 2B). A significant elevation of urinary albumin excretion (UAE) was also observed only at the 12 month timepoint (FIG. 2C). Light microscopic images of NT and bGH kidney sections show glomerular hypertrophy in the bGH kidney sample along with a thickened Bowman's capsule. The observed changes seen in the bGH mice are indicative of kidney damage.

Decreased KAP mRNA Expression in the Kidneys of MT/bGH-Transgenic Mice

In an effort to identify genes involved in the kidney damage resulting from expression of the bGH transgene, we performed a PCR-based method of subtractive hybridization using RNA extracted from kidneys of 12-month-old MT/bGH-transgenic mice and their nontransgenic littermates. Two cDNA subtraction libraries were created, one (bGH) containing cDNAs up-regulated in the MT/bGH kidney in comparison to the nontransgenic kidney, and one (NT) containing cDNAs down-regulated in the MT/bGH kidney. A cDNA exhibiting high sequence similarity to the KAP nucleotide sequence was isolated from the library of down-regulated cDNAs. Northern analysis confirmed the decreased expression in the MT/bGH kidney as a function of age that correlated with degree of kidney damage.

Research Design and Methods

We have demonstrated that KAP mRNA expression is inversely correlated with kidney damage (i.e. KAP expression decreases with increasing kidney damage). This decrease in KAP gene expression could either be the cause of the kidney damage or could result from the kidney damage. One way to test the former possibility would be to knock out expression of the KAP gene and assess whether kidney damage occurs independently of diabetes. In a converse experiment, high levels of KAP expression could be maintained and the kidneys assessed for protection from damage when diabetes is induced. The second experiment would further support KAP as a therapeutic drug for the prevention of diabetic nephropathy if shown to be protective while the first experiment would further support KAP as a drug target for the prevention of kidney damage. Due to the practical outcome of the second experiment, we propose to test the hypothesis that maintenance of high levels of KAP expression will protect the kidney from glomerular hypertrophy and diabetic damage. To do this, we will create a transgenic mouse line that expresses KAP in the kidney under the direction of a heterologous promoter such that KAP expression will no longer decrease with increasing kidney damage. We will then challenge the kidney by inducing diabetes in the KAP-transgenic mouse and assessing the degree of kidney damage. The hypothesis will be considered proven if the kidneys of diabetic KAP-transgenic mice more closely resemble nondiabetic rather than diabetic nontransgenic control kidneys. Confirmation of the hypothesis would further support of KAP as a therapeutic drug to prevent diabetic nephropathy.

Specific Aim 1: To Constitutively Express KAP mRNA in the Mouse Kidney

Rationale

It has been demonstrated that KAP mRNA levels decrease with increasing kidney damage. In order to prevent this decrease in expression, the KAP cDNA will be placed under the control of a heterologous promoter that should not be affected by the state of diabetes (use of the term “promoter” in this proposal includes the region of the gene required to initiate transcription as well as regions that regulate expression). Although a number of genes are expressed in the renal proximal tubule, very few have been shown to be expressed only in those cells. Furthermore, very few of the promoters of these genes have been characterized well enough to demonstrate cell-specific expression of a reporter gene. A search of the literature has indicated two different promoter regions that may provide suitable transcriptional-regulation. The first, a 346-base pair region from the type II promoter of the mouse gene encoding gamma-glutamyl transpeptidase (GGT), has been shown to direct expression of a β-galactosidase reporter gene specifically in the renal proximal tubules of transgenic mice (Sepulveda et al., 1997). Although the GGT promoter has the correct tissue-specific expression, there is some question as to whether the level of expression will be adequate. The second promoter selected is from the mouse metallothionein-I gene. Although it was reported to direct expression of human growth hormone-releasing hormone in renal proximal tubules, it also directed expression in a number of other tissues, including pancreas, lung, heart and brain (Brar et al., 1989). Since an “ideal” promoter has not been found, both of these promoters will be used and the results compared.

Experimental Design

a) Construct a plasmid fusing the KAP cDNA to a heterologous promoter that will drive KAP expression in the kidney. An expression plasmid routinely used in our laboratory will serve as a starting point for constructing the heterologous promoter/KAP cDNA fusion. This plasmid utilizes the mouse metallothionein-I promoter to initiate transcription. The plasmid also has a bGH polyadenylation signal sequence that defines the site of poly A addition. The 611 bp cDNA for KAP, isolated from our subtraction libraries, will be inserted between the promoter and the polyadenylation signal sequence, generating the MT/KAP fusion construct. Nucleotide probes complementary to the bGH sequence just upstream of the poly A addition site will be used to distinguish transgenic KAP mRNA expression from endogenous KAP mRNA expression. A protein tag will not be added to the protein sequence since, without a known function for KAP, it will be impossible to know if the tag interferes with function. The plasmid also has a bacterial origin of replication and an ampicillin resistance gene for propagation in E. coli. Replacement of the MT promoter region with the 346 bp GGT promoter. (Sepulveda et al., 1997) will generate the GGT/KAP fusion-construct. All manipulated regions of the expression plasmids will be sequenced to confirm that no errors were introduced.

b) Confirm mRNA and protein expression from the fusion construct in cell culture. Before injection into mice, expression from each KAP fusion construct will be confirmed in cell culture. Mouse L cells, used routinely in our laboratory, will be transiently transfected with the KAP fusion constructs using lipofectin. Transfected and nontransfected control cells will be harvested and either RNA or protein extracted. RNA will be analyzed by Northern blot hybridization as described in the Preliminary Results section using a probe complementary to the bGH sequence at the 3′ end of the KAP transgene message. Protein will be analyzed for KAP expression by Western blot analysis using monoclonal antibodies as described by Cebrian et al., 2001 (2001). Since a protein tag will not be utilized for the transgene, protein expression paralleling the transgenic mRNA expression and differing from normal expression will be evaluated. In the event that the GGT promoter is not functional in mouse L cells, a mouse proximal tubule cell line will be tried (Sepulveda et al., 1997). Our laboratory has considerable experience performing Northern and Western blot analysis.

c) Create transgenic mouse lines carrying the KAP transgene. The pronuclear microinjection technique into single-cell fertilized embryos will be utilized to deliver the KAP transgene into the mouse genome. Our transgenic mouse facility has been using the technique since the mid-1980s (McGrane et al., 1988). Since then, our facility has optimized the technique and created hundreds of transgenic lines for Ohio University researchers as well as outside researchers. Pups obtained from the microinjections will be screened for the presence of the KAP transgene by DNA slot blot and Southern blot hybridization. The number of integrated KAP transgene copies will be quantitated by comparison to known copy number standards. Transgenic lines will be established by crossing the transgenic founders to nontransgenic mice and inheritance of the KAP transgene confirmed by DNA slot blot hybridization. These techniques are performed routinely in our laboratory.

d) Confirm mRNA and protein expression from the fusion construct in the KAP-transgenic mouse lines. F1 progeny from the KAP transgenic lines will be assayed for KAP transgene expression. Transgenic mice and their nontransgenic littermates will be sacrificed by cervical dislocation and several organs (kidneys, pancreas, lung, heart and brain) immediately frozen. RNA or protein will be extracted from the tissues and then analyzed by Northern or Western blot analysis as described above. Tissues other than kidney will be included in order to determine the degree of tissue specificity. Lines expressing the highest levels of KAP from the transgene will be expanded for further analysis (see below).

Analysis

The experiments proposed for Specific Aim 1 should result in generation of two different transgenic mouse lines that express KAP in renal proximal tubules. However, several potential problems could be encountered. First, the selected promoters may not direct sufficient or appropriate expression. One way to increase expression from the MT promoter is to supplement the mouse drinking water with ZnSO₄ (Eisen et al., 1998). Other promoters that could be considered include the mouse phosphoenolpyruvate carboxykinase (PEPCK) promoter or the cytomegalovirus (CMV) promoter. Although the PEPCK promoter has been shown to direct expression in renal proximal tubules, as well as other tissues (Short et al., 1992), it was questionable as to whether induction of diabetes would influence its ability to direct transcription. Likewise, although CMV does direct expression in the kidney, it was not clear whether expression occurred in the proximal tubules.

Another potential concern is use of the KAP cDNA for expression. Sometimes we find that inclusion of an intron increases expression. Alternative constructs could include the 126 bp second intron of KAP or the 3807 bp genomic clone (Niu et al., 1991).

Specific Aim 2: To Induce Diabetes in the KAP-Transgenic Mice and Assess the Degree of Kidney Damage

Rationale

In order to determine if KAP mRNA expression (and subsequent protein production) can protect the kidneys of diabetic mice from damage, diabetes will be induced in the KAP-transgenic mice generated under Specific Aim 1. After 4 to 10 weeks of diabetes, diabetic and nondiabetic KAP-transgenic and nontransgenic mice will be sacrificed and their kidneys examined. If the parameters measured for the diabetic KAP-transgenic mice (glomerular volume, mesangial cell expansion, and urinary albumin excretion) are significantly different from diabetic nontransgenic mice and in fact more closely resemble those for nondiabetic, nontransgenic mice, then the hypothesis will be considered proven with evidence suggesting the use of KAP as a therapeutic drug for the prevention of diabetic kidney damage.

Experimental Design

Diabetes will be induced in twenty-five 2-month-old KAP-transgenic and nontransgenic littermate females by multiple injection of STZ as described in the Preliminary Results section. Nondiabetic controls (twenty KAP-transgenic and twenty nontransgenic littermates) will be injected with citrate buffer only. After 4 or 10 weeks of hyperglycemia (blood glucose levels 300-500 mg/dl), urine will be collected from diabetic KAP-transgenic mice and the three control groups (diabetic nontransgenic, nondiabetic KAP-transgenic and nondiabetic nontransgenic; 8-10 mice/group). The mice will be sacrificed by cervical dislocation and tissues (kidneys, pancreas, lung, heart, brain, and liver) immediately removed. The right kidney will be quickly frozen for later RNA and protein extraction and the left kidney will be bisected and placed in 4% paraformaldehyde. RNA and protein will be examined by Northern and Western blot analyses to determine levels of transgenic and endogenous KAP expression as described above. Glomerular volume, mesangial cell expansion and UAE will be measured as described in the Preliminary Results section. Statistically significant differences will be determined by ANOVA.

Analysis

Performance of Specific Aim 2 should establish whether expression of KAP in kidney using a heterologous promoter can protect the kidney from diabetic damage. If KAP expression protects the kidney, then the measurements of glomerular volume, mesangial cell expansion and UAE should not differ significantly between diabetic, KAP-transgenic mice and nondiabetic, nontransgenic mice. Instead, there should be a significant difference between diabetic, KAP-transgenic mice and diabetic, nontransgenic mice.

One potential problem that might be encountered is an effect of diabetes on expression by the heterologous promoters. Northern and Western blot analyses of several different tissues, including kidney, should help determine if this is a problem. Also, it is unlikely that both promoters will be affected.

Another possible scenario is that expression of the KAP-transgene will affect the measured kidney damage parameters (glomerular volume, mesangial cell expansion and UAE) differently. For example, diabetic, KAP-transgenic mice may no longer exhibit mesangial cell expansion and increased UAE (parameters that typically change during the later stages of diabetes in diabetic, nontransgenic mice; see Preliminary Results section) but may exhibit glomerular hypertrophy. This differential effect may help pinpoint the role of KAP in the kidney. Interestingly, in contrast to our results, KAP mRNA was reported as being slightly increased (1.5×) in remnant kidneys exhibiting glomerular hyperfiltration two weeks after undergoing subtotal renal ablation (5/6 nephrectomy) (Zhang et al., 1999). A possible explanation is that KAP expression increases during the very early stages of diabetes (prior to our earliest timepoint, perhaps in an effort to protect the kidney) but then decreases as diabetes progresses.

Another potential problem is that the heterologous promoters may not be able to drive sufficient expression of KAP for it to protect against diabetic damage. KAP appears to be highly expressed in our female mice, as shown in the Preliminary Results section. We have not assayed KAP expression in our male mice, but reports in the literature indicate that KAP expression is highest in males and lower in females and castrated males (Cebrian et al., 2001). Achievement of even higher levels of KAP may require characterization of a similarly expressed gene promoter, such as that for the gamma phosphorylase kinase gamma-subunit (Takenaka et al., 1998).

The possibility also exists that increased KAP expression will not protect the kidney from damage, but that the decreased expression results from the development of kidney damage. If this appears to be the case, then decreased KAP expression could potentially be used as a diagnostic marker for kidney damage.

Future Direction

As demonstrated in the Preliminary Results section, KAP mRNA expression also decreases with increasing kidney damage as a function of age in MT/bGH-transgenic mice. Since these mice do not become diabetic, their kidney damage presumably occurs via a different mechanism. Thus, they provide an independent model for the study of progressive glomerular hypertrophy and glomerulosclerosis. Although completion lies beyond the time frame of this proposal, we hope to initiate a study that combines the bGH transgene and the KAP transgene in a double-transgenic mouse through genetic breeding. The double-transgenic mice, and single-transgenic controls, will then be sacrificed at 2, 5, or 12 months of age and their kidneys examined for KAP transgenic and endogenous expression and for glomerular hypertrophy and mesangial cell expansion as described for Specific Aim 2. Data demonstrating that KAP expression protects the kidney from damage due to the bGH transgene would further support and even broaden the role of KAP in protecting the kidney from damage.

Adaptation to Human Therapy

To adapt the gene therapy system for humans, we could use somatic cell gene therapy. Any vector that delivers DNA into the nucleus (i.e. viral or non-viral methods) as well as any promoter that could direct constitutive, high levels of expression could potentially be used. Since KAP may be a secreted protein, it may not be important to direct expression only to kidney cells. Although we anticipate that NAP would protect the kidneys, it could possibly protect other organs as well. KAP would preferably be administered/maintained as long as the patient is diabetic and susceptible to kidney damage. Administration route could be any of the following: subcutaneous, intravenous, intramuscular.

KAP could potentially be used in humans as a “conventional” pharmaceutical, especially it is a secreted protein and thus would normally be found circulating throughout the body. The administration route could be any that would keep the protein intact (e.g. subcutaneous, intravenous, intramuscular).

Literature Cited for Example 201

Bellush, L. L., Doublier, S., Holland, A. N., Striker, L. J., Striker, G. E., and Kopchick, J. J. (2000). Protection against diabetes-induced nephropathy in growth hormone receptor/binding protein gene-disrupted mice, Endocrinology 141, 163-8.
Brar, A. K., Brinster, R. L., and Frohman, L. A. (1989). Immunohistochemical analysis of human growth hormone-releasing hormone gene expression in transgenic mice, Endocrinology 125, 801-9.

Cebrian, C., Areste, C., Nicolas, A., Olive, P., Carceller, A., Piulats, J., and Meseguer, A. (2001). Kidney androgen-regulated protein interacts with cyclophilin B and reduces cyclosporine A-mediated toxicity in proximal tubule cells, J Biol Chem 276, 29410-9.

Chen, W. Y., Wight, D. C., Wagner, T. E., and Kopchick, J. J. (1990). Expression of a mutated bovine growth hormone gene suppresses growth of transgenic mice, Proc Natl Acad Sci USA 87, 5061-5.

Chin, E., Zamah, A. M., Landau, D., Gronbcek, H., Flyvbjerg, A., LeRoith, D., and Bondy, C. A. (1997). Changes in facilitative glucose transporter messenger ribonucleic acid levels in the diabetic rat kidney., Endocrinology 138, 1267-75.

Condorelli, G., Vigliotta, G., Iavarone, C., Caruso, M., Tocchetti, C. G., Andreozzi, F., Cafieri, A., Tecce, M. F., Formisano, P., Beguinot, L., and Beguinot, F. (1998). PED/PEA-15 gene controls glucose transport and is overexpressed in type 2 diabetes mellitus, Embo J 17, 3858-66.

Eisen, E. J., Peterson, C. B., Parker, I. J., and Murray, J. D. (1998). Effects of zinc ion concentration on growth, fat content and reproduction in oMT1a-oGH transgenic mice, Growth Dev Aging 62, 173-86.

Esposito, C., Liu, Z. H., Striker, G. E., Phillips, C., Chen, N. Y., Chen, W. Y., Kopchick, J. J., and Striker, L. J. (1996). Inhibition of diabetic nephropathy by a GH antagonist: a molecular analysis, Kidney Int 50, 506-14.

Hill, C., Flyvbjerg, A., Gronbaek, H., Petrik, J., Hill, D. J., Thomas, C. R., Sheppard, M. C., and Logan, A. (2000). The renal expression of transforming growth factor-beta isoforms and their receptors in acute and chronic experimental diabetes in rats., Endocrinology 141, 1196-208.

Kasik, J. W., and Rice, E. J. (1993). Kidney androgen-regulated protein gene is expressed in the uterus during late pregnancy, Mol Cell Endocrinol 90, 239-42.

McGrane, M. M., de Vente, J., Yun, J., Bloom, J., Park, E., Wynshaw-Boris, A., Wagner, T., Rottman, F. M., and Hanson, R. W. (1988). Tissue-specific expression and dietary regulation of a chimeric phosphoenolpyruvate carboxykinase/bovine growth hormone gene in transgenic mice, J Biol Chem 263, 11443-51.

Meseguer, A., and Catterall, J. F. (1987). Mouse kidney androgen-regulated protein messenger ribonucleic acid is expressed in the proximal convoluted tubules, Mol Endocrinol 1, 535-41.

Meseguer, A., and Catterall, J. F. (1990). Cell-specific expression of kidney androgen-regulated protein messenger RNA is under multihormonal control, Mol Endocrinol 4, 1240-8.

Meseguer, A., and Catterall, J. F. (1992). Effects of pituitary hormones on the cell-specific expression of the KAP gene, Mol Cell Endocrinol 89, 153-62.

Meseguer, A., Watson, C. S., and Catterall, J. F. (1989). Nucleotide sequence of kidney androgen-regulated protein mRNA and its cell-specific expression in Tfm/Y mice, Mol Endocrinol 3, 962-7.

Niu, E. M., Meseguer, A., and Catterall, J. F. (1991). Genomic organization and DNA sequence of the mouse kidney androgen-regulated protein (KAP) gene, DNA Cell Biol 10, 41-8.

Page, R., Morris, C., Williams, J., von Ruhland, C., and Malik, A. N. (1997). Isolation of diabetes-associated kidney genes using differential display, Biochem Biophys Res Commun 232, 49-53.

Peraldi, M. N., Berrou, J., Hagege, J., Rondeau, E., and Sraer, J. D. (1998). Subtractive hybridization cloning: an efficient technique to detect overexpressed mRNAs in diabetic nephropathy, Kidney Int 53, 926-31.

Quaife, C. J., Mathews, L. S., Pinkert, C. A., Hammer, R. E., Brinster, R. L., and Palmiter, R. D. (1989). Histopathology associated with elevated levels of growth hormone and insulin-like growth factor I in transgenic mice, Endocrinology 124, 40-8.
Sepulveda, A. R., Huang, S. L., Lebovitz, R. M., and Lieberman, M; W. (1997). A 346-base pair region of the mouse gamma-glutamyl transpeptidase type II promoter contains sufficient cis-acting elements for kidney-restricted expression in transgenic mice, J Biol Chem 272, 11959-67.

Short, M. K., Clouthier, D. E., Schaefer, I. M., Hammer, R. E., Magnuson, M. A., and Beale, E. G. (1992). Tissue-specific, developmental, hormonal, and dietary regulation of rat phosphoenolpyruvate carboxykinase-human growth hormone fusion genes in transgenic mice, Mol Cell Biol 12, 1007-20.

Song, Y., Ailenberg, M., and Silverman, M. (1998). Cloning of a novel gene in the human kidney homologous to rat munc13s: its potential role in diabetic nephropathy, Kidney Int 53, 1689-95.

Striker, G. E., Eastman, R. D., and Striker, L. J. (1996). Diabetic nephropathy: molecular analysis or extracellular matrix and clinical studies update, Nephrol Dial Transplant 11, 58-61.

Takenaka, M., Imai, E., Kaneko, T., Ito, T., Moriyama, T., Yamauchi, A., Hori, M., Kawamoto, S., and Okubo, K. (1998). Isolation of genes identified in mouse renal proximal tubule by comparing different gene expression profiles, Kidney Int 53, 569-72.

Unger, R. H., and Foster, D. W. (1998). Diabetes mellitus. In Williams Textbook of Endocrinology, J. D. Wilson, D. W. Foster, H. M. Kronenberg, and P. R. Larsen, eds. (Philadelphia, W. B. Saunders Company), pp. 973-1059.

Virlon, B., Cheval, L., Buhler, J. M., Billon, E., Doucet, A., and Elalouf, J. M. (1999). Serial microanalysis of renal transcriptomes, Proc Natl Acad Sci USA 96, 15286-91.

Wada, J., Zhang, H., Tsuchiyama, Y., Hiragushi, K., Hida, K., Shikata, K., Kanwar, Y. S., and Makino, H. (2001). Gene expression profile in streptozotocin-induced diabetic mice kidneys undergoing glomerulosclerosis, Kidney Int 59, 1363-1373.

Wardle, E. N. (1996). Glomerulopathies: Cell biology and immunology (Amsterdam, Harwood Academic Publishers).

Wirta, O., Pasternack, A., Laippala, P., and Turjanmaa, V. (1996). Glomerular filtration rate and kidney size after six years disease duration in non-insulin-dependent diabetic subjects, Clin Nephrol 45, 10-7.

Yang, C. W., Striker, G. E., Chen, W. Y., Kopchick, J. J., and Striker, L. J. (1997). Differential expression of glomerular extracellular matrix and growth factor mRNA in rapid and slowly progressive glomerulosclerosis: studies in mice transgenic for native or mutated growth hormone, Lab Invest 76, 467-76.

Yang, C. W., Vlassara, H., Peten, E. P., He, C. J., Striker, G. E., and Striker, L. J. (1994). Advanced glycation end products up-regulate gene expression found in diabetic glomerular disease., Proc Natl Acad Sci USA 91, 9436-40.

Zhang, H., Wada, J., Kanwar, Y. S., Tsuchiyama, Y., Hiragushi, K., Hida, K., Shikata, K., and Makino, H. (1999). Screening for genes up-regulated in 5/6 nephrectomized mouse kidney, Kidney Int 56, 549-58.

Ziyadeh, F. N., Cohen, M. P., Guo, J., and Jin, Y. (1997). RAGE mRNA expression in the diabetic mouse kidney, Mol Cell Biochem 170, 147-52.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g, mutually exclusive choices for an element of the invention) or which are expressly excluded by this specification.

If an embodiment of this invention is disclosed in the prior art, the description of the invention shall be deemed to include the invention as herein disclosed with such embodiment excised.

TABLE 1

Sequence of Clone G26, related to hydroxysteroid
dehydrogenase-4

1A: beginning of coding strand (SEQ ID NO:1)

actcagctcctacnggntgcttgtgtggaagccaacgtgccaacattcat
ctacagcagctcagtgcttgtggctggaccaaattcctacaaggagatca
tcctgaatggccatgaggaggagcatcatgaaagcacatggtctaaccca
tacccatgnnnngtgaaagatggctgagaaggcagtgctggcaacccntg
ggagcatcctgaaaaatggtggcactttgcatacttgtgccttaagactc
tctttcatctatggggaagaatgccaagtcacttcaaccactgtgaaaac
agcactgaagaacaacagcataattaagaaagatgccacattctccattg
ccaacccagtgtatgtgggcaatgcancctgggctcacattctggctgcc
aggagcctacaggaccctaaaaagtccccaagcatccaaggacagttcta
ttacattaacagatgacacccctcaccaaagcnatgatgacttnaaaatg
caccctgagcaaggagtgggggcccncccccttganaccaactgggaagt
ctttcctctgccccctgctntactnggcnttgccttncctccccnggaaa
actggggaaccttcctgctaccnnccannctaccaacttannnaaccccn
ttttaaaccccccnttggaccnccanngnnaaaaaaangnnntncncccn
ntcctaccaaaaaggcncaacnnnnaacnnggcnttnancnnnttng
1B: end of coding strand, obtained by sequencing
opposite strand, deducing complement, and
arranging in 5′-3′ order (SEQ ID NO: 2)
ngggcttncggcccnnananntcccaagcnnccaggnnggtttnttcctt
tcnangnncccctcaccaaaagtatgntnatttaaaagacccttgancaa
agantggggccttngctttgatacanntggagtntttctntggcccnnct
ncnatnggcntgctttcntcttnaaactntgagnntccnggntacgtcca
gtnttncaaaataaaancccgcccatttaaccgcctcttgatcacagtgn
ttaaanagtgatccacctctcctanaagaaggtcagngagatttgggcta
tgagccaacttgtcagctgggaggnagccaagcagaaaacctcagagtgg
atcggaaacactagtgatgcagcacagggagattggaaacaaaaagtcnc
agtgatatgaagagggagaggacatggccctgggtgttattaggtcctcc
agaaagggacttagaaacaatccaactcttaacaattccattttacactc
tgtccaacttgtctttgggtcaccagaagccttgcaagtcactggcccag
ttgcaacccttcagctntaagcacttgtccagcaatgcacaagatgtgcc
tcagctgcngtgacccaaggatgggtggctgatagtgagttgcctggaac
ctcttgtaggttagaatttcatcnnggcttccacatctcctttccatgtg
ccaacgcatttcgtgtctgagaaaattcccatacctttatgaagctcaaa
gaatagaacacataaaatcttttaatgcgtaaaaaaaatnaaaaaaaaa

TABLE 2

Sequence of Clone H8 (SEQ ID NO:3), related to
vacuolar adenosine triphosphatase subunit D:

acttcaatgtggaccatggctacctggagggcctggttcgaggatgcaaa
gccagcctcctaactcagcaggactatgtcaacctagtgcagtgtgagac
ctcggaagacctgaaaattcatctccagaccacggactatggcaacttcc
tggctaatgaaacaaatcctctcactgtttccaaaattgacacggagatg
aggaagaagttctgcagagagtttgactatttccggaatcattccttgga
gtccctgagcacatttctcacctacatgacatgcagctatatgatagaca
atataattctacttatgaatggggccttgcaaaagaaatctgtgaaagaa
gttctagccaagtgtcacccactgggccgtttcacagagatggaagctgt
caacattgcagagaccccctcagatctcttcaggcttgtgctggttgaaa
caccattagctccattctttcaagattgtatgtcttgaaaacacctcttg
atgaacttgaatattgaattactgcgcaataaactatacaagtcttacct
tgaggcattctacaaattctgcaaggatcacggtgatgtcacagcagacg
ttatgtgtcccattcttgagtttgaggccgacagacgcgctttaatcatc
actctgaactcatttggcactgaactaagcaaagaagacagggagaccct
cttccccacctgcggcaggctctatccagaggggttgcggttgttagctc
aagctgaagactttgagcagatgaagagagtggcagataattatggagtt
tacaagcctttgtttgactgctgtcggtggcagtggggggaagacactgg
aagacgttttctatgagagagaggt

TABLE 3

Sequence of Clone H1 (SEQ ID NO:4), related to
ubiquitin protein ligase Nedd-4

acatacgtgtttacggagttcattatgtttacagattaagcgaatttctg
tagttgcatttttatatttttagtatcacattagtataaaaatttgttta
aaatagccaaagagtagttcaatgcataatttgtatgaatttgttaccaa
gtttcttatgctttctaaaaaatactgtttcctatgaaaattatgtttaa
tcaaaagtcaagaacccttggggcaaaatgctacaggtggagtcccacca
tagtcacctttgaggcacaggaagaggttccatgcatgaatctgcacaca
tgagcagtgctggctgctgtgcaccgggcccttgggatgaagccatcagc
actgacatccagccccgtcttcatcctcaggcttagaaacaagtgggatg
gtgactttacagtcagtgggaagtaagagcccgcagaataacaaaggcag
aaggacaggaaagcgaggtccgagtccgactgtgaggtcatatctggcca
aggctccttctagtcagcttacactggtgattatacggaaaatgctctta
gatatgagaaaagcaatgtggcatgtaatgagttagtgtaagcttgtgtt
ataaactttccgaatgcttataagtagcctgtgggagctgtaagaaatac
ttagggccattgatttaagcttgttggctctaacatgactaatacctgtc
agcccatggtagggatttcagtgacagtagtggaaacgctttccacagtc
cacaccactatgttgatcggtgaaagacacccccaggccagtgcatgctt
ccagagtgtcctgatcagtcagccaggcatggccaccgtccagtgttgat
ttttatcattagccagctgagcctgcagcacggggctcatttccccgtct
ggcagatgtggatagctgggcccactcattctgtctctcaaggctatggt
aagttcttaaaggggt

TABLE 4

Sequence off Clone F1 (SEQ ID NO:5), related to
truncated SON protein.

acaagcccgacttgaaagaggcctccagaaaaagtagatgcgtatctgtg
caaacagatcctactgatgaagtgcccaccaaaaagtcaaagaagcataa
aaagcacaaaaacaaaaagaagaaaaagaagaaagaaaaagaaaaaaaat
ataaaagacaaccagaagaatctgagtccaagctgaaatctcatcatgat
gggaatctagaatctgattccttcttaaagtttgattctgaaccttcagc
agcggcactggaacatcctgtaagagcgtttggcttatctgaggccagtg
agaccgccttagtgctggaacctccagtagtctcaatggaggttcaggag
tcacacgttctagagactctgaagccagctacaaaagctgcagaactgtc
agttgtgtctacatcagtaatctcagagcagtctgagcagccgatgccag
gtatgctggaaccatcaatgacaaagatcctggattccttcacagcagca
ccagtggcctatgtcaacagcggcgctgaagtcacctgagccccgttgta
acaaatgtcagtggaatatcagaagtctgtgctgaaatctttggagacta
tgcctccagagacgtcaaagaccacgctggtagagcttcccatagcaaaa
gtggttgagccatcagaaaccctcacgatagtgtcagagacacctactga
ggtgcaccctgaaccaagcccatcaacaatggattttccagagtcatcta
caactgacgtgcaaagattgccagagcagcctgtagaagcaccatcggag
attgcagattcatccatgacaagacctcaggagtcactggagctgcctaa
gaccacagcggtggagctgcaggagtcaacggtggcctcagctctggagt
tgccggggccacctgcgacctccattctggagttgcaggggccccctgtg
actccagtgccagagttgcctgggccctctgccaccccagtgccagagtt
gtcagggcccctttctaccccagtgcctgagttgccagggccccctgcga
cagtggtccctgagttgccggggccctctgtgacaccagtgccacagttg
tcgcaggaattgccagggcctccagcaccatccatggggttggagccacc
acaggaggt

TABLE 5

Sequence of Clone F4 (SEQ ID NO:6), related to
kidney androgen regulated protein

acgcggggtaatttctttctcctgitcactggttgccttaaccctactaa
agcatgatgcttttcaaggtcctgatgatcactgtctcctgtaatctgac
tgtggctttccccctgtcagaattagtttcaatcaataaagaactacaga
attcaatcattgacctactaaactcagtctttgaccaactgggatcatac
agagggacaaaagctcctctagagaattatacagatgatgatttaagcac
tgactctgagcagatcatggacttcacgccagccgcaaacaaacagaatt
ctgagttctctactgatgttgagacagtctcctccggctttctggaagaa
ttcactgagaacacagacatcacagtgaaaattccattagctgggaatcc
agtctcccctacttcctgagaagactccaactttgaaataaacctgcgac
tgccattcaggatattgtctttgtagaaaatgatataaatctaagatgct
cccacaaagaagaaagaacgaggaagtgatcctggatgagaaatgctgtg
ccaggcatctctaagctctacccttactntgtgtgggtttaagaaataaa
ccgttcatttgattgc

TABLE 6

Sequence off Clone F5 (SEQ ID NO:7), related to
claudin-10

acgcggggnagtagggaattagaaagattattctaagctatctctaagat
gcccttagtgtgatatactttattctaatttcccacacttcaagccatga
gattatgaagaattgtcacctaccctcatgggctggctttaacattcgtg
gaagttttgtccagtgtctgcatgcctaggccaccaaagccttctgtgtg
gactgtggcagcaggcaaggctgagcgacatgtccagggcacagatctca
gctctggtgtgtggtgttggagggtttggtgctctcgtcgctgccaccac
atccaacgaatggaaagtgaccacccgagcgtcgtctgtgattaccgcca
cctgggtttaccagggtctgtggatgaactgcgcaggtaacgctctgggc
tccttccactgccggccacatttcactatcttcaaagtagaaggttacat
ccaggcatgtagaggactaatgatcgctgcggtcagcctgggatttttcg
gttccatttttgcactctttggaatgaaatgtaccaaagtcggaggctca
gatcaagccaaagctaaaatccgcttgctttggcccgggattggattcat
attgncaggtctggggttccatgacaggctgttcccttgtntgcaaccaa
atcncaacngaattctttgatcctntttatatggagcaaaagtttgaatt
aggggcttgtttttttatcggatgggcngaacttttttntgntcatgggg
gagnatatttggttttaatatccacacaccaacnccaaaagggtacnntc
aanggncccnttngcatgtttttggaccagtttaggngnaaagaaaaaaa
aaaaaaaaaaancctngccnancctagggan

TABLE 7

Sequence of Clone F6 (SEQ ID NO:8), related to
kidney androgen regulated protein

aacttctttctcctgttcactggttgccttaaccctactaaagcatgatg
cttttcaaggtcctggtgatcactgtctcctgtggtctgactgtggcttt
ccccctgtcagaattagtttcaatcaataaagaactacagaattcaatca
tcgacctactaaactcagtctttgaccaactgggatcatacagagggaca
aaagctcctctagaggattatacagatgatgatttaagcactgactctga
gcagatcatggacttcacgccagccgcaaacaaacagaattctgagttct
ctactgatgttaagacagtctcctccggctttctggaagaattcactgag
aacacagacatcacagtgaaaattccattagctgggaatccagtctcccc
tacttcctgagaagactccaactttgaaataaacctgcgactgccattca
ggatattgtctttgtagaaaatgatataaatctaagatgctcccagcaaa
gaagaaagaacgaggaagtgatcctggatgagaaatgctgtgccaggcat
ctctaagctctacccttactctggggtgggtttaagaaataaacgttcat
tttgaattccc

TABLE 8

Sequence of Clone A8 (SEQ ID NO:9) related to an
immunoglobulin sequence

acgcgggtgagcagcaccctcacattgaccaaggacgagtatgaacgaca
taacagctatacctgtgaggccactcacaagacatcaacttcacccatcg
tcaagagcttcaacaggaatgagtgttagagccaaaggtcctgagacgcc
accaccagctccccagctccatcctatcttcccttctaaggtcttggagg
cttccccacaagcgacctaccactgttgcggtgctccaaacctcctcccc
acctccttctcctcctccctttacttggcttttatcatgctaatatttgc
agaaaatattcaataaagtgagtctttgcacttg

TABLE 9

Sequence of Clone A34 (SEQ ID NO:10), related to
an immunoglobulin sequence

acgttgtaaaacgacggccagtgaattgtaatacgactcacttatagggc
gaattgggccctctagatgcatgctcgagcggccgccagtgtgatggata
tctgcagaattcgccctttcgagcggccgcccgggcaggtacagtgtggg
tttaccagtggacttgtccacggtcctctcggtcaccaggtgtggcaggg
cctcgtggcctacaacacaggtataggtctctccggagttccattcctcc
tctgtcacagtcaggatgctgtgggtaaagtagaagcctggggccccagg
ctctggcatcggggcactggtcacatacttctcttggggcaagagttgcc
ctccctgaagccactgcacactgatgtctgcaggagagaagcccttcacc
aggcaggtgactgtggctgactccctcaggttcagttgctcacgagctgg
tggcagcaggtacctcggccgcgaccacgctaagggcgaattccagcaca
ctg

TABLE 10

Sequence of Clone A39 (SEQ ID NO:11),
related to an immunoglobulin sequence

acaagatggggactgggacgggcctttgactactggggccaaggcaccac
tgtcacagtctcctcagccaaaacaacacccccatcagtctatccactgg
cccctgggtgtggagatacaactggttcctctgtgactctgagatgcctg
gtcaagggctacttccctgagtcagtgactgtgacttggaactctggatc
cctgtccakcagtgtgcacaccttcccagctctcctgcagtctggactct
acactatgagcagctcagtgactgtcccctccagcacctggccaagtcag
accgtcacctgcagcgttgctcacccagccagcagcaccacggtggacaa
aaaacttgagcccagcgggcccatttcaacaatcaacccctgccctccat
gcaaggagtgtcacaaatgcccagctcctaacctcgagggtggaccatcc
gtctttatcttccctccaaatatcaaggatgt

TABLE 11

Sequence of Clone A48 (SEQ ID NO:12),
related to an immunoglobulin sequence

acaccattccacctcccaaggagcagatggccaagaataaaatcaatccg
acctacatgataacaaacttcttccctgaagacattactgtggagtggta
gtggaatgggcagccagcggagaactacaagaacactcagcccgtcacgg
acacagatggctcttacttcgtctacagcaagctcaatgtgcagaagagc
aactgggaggcaggaaatactttcacctgctctgtgttacatgagggcct
gcacaaccaccatactgagaagagcctctcccactctcctggtaaatgat
cccagtgtccttggagccctctggtcctacaggactctgacacctacctc
cacccctccctgtgtaaataaagcacccagcactgccttgggaccctgca
aaaaaaaaaaaaaaaaaaaaaaaaaaaagt

TABLE 12

Sequence of Clone B45 (SEQ ID NO:13),
related to kidney androgen related protein

acgcggggaatttctttctcctgttcactggttgccttaaccctactaaa
gcatgatgcttttcaaggtcctggtgatcactgtcttctgtgatctgact
gtggctttccccctgtcagaattagtttcaatcaataaagaactacagaa
ttcaatcattgacctactaaactcagtctttgaccaactgggatcataca
gagggacaaaagctcctctagaggattatacagatgatgatttaagcact
gactctgagcagatcatggacttcacgccagccgcaaacaaacagaattc
tgagttctctactgatgttgagacagtctcctccggctttctggaagaat
tcactgagaacacagacatcacagtgaaaattccattagctgggaatcca
gtctcccctacttcctgagaagactccaactttgaaataaacctgcgact
gccattcaggatattgtctttgtagaaaatgatataaatttaagatgctc
ccagcaaagaagaaagaacgaggaagtgatcctggatgagaaatgctgtg
ccaggcatctttaagctctgcccttactctgtgtgggtttaagaaataaa
cgttcatttg

TABLE 13

Sequence of Clone C22 (SEQ ID NO:14),
related to disabled −2 p96 (Dab2)

actgtgtaggtttccaaggctatgaggaatatatttcaggcagatttttc
aatgataaccccaaatatttgtccatgattgtggcttcaagaanttttcc
agttacttgataaaggcctagtcatccagtaaggacccagtgtgtctagc
atagtcacatttagctttcagaatacagtccttcactgctgagcagaatc
cagttcccctactgtgttaaattgctctatgaaacttcagtctaagtccc
ccaagccatagggagaatgaagaaggtgcaccttccagaagtctagcccc
tggactaacaatgactggagtgggtgcgtgcacttcagatctatgaaaca
tttaatgctttttgccccgtgaaaaatccattcctggtaaaagggaggca
ctgtttatataaacccaggagcaaaatgaactg

TABLE 14

Sequence of Clone E39, related to an
immunoglobulin sequence

14A: beginning of coding strand (SEQ ID NO:15)

acgcgggggagccacacaaactcagggaaagctcgaatatggttttcaaa
cctcagatacttggacttatgctttttaggatttcagcctccagaggtga
tattggctacctcaatctccagccaccctgtctgtgcctccaggagatag
cgtcagtctttccttcagggccagccaaaatattagcaacaacctacact
gggttcaaaaaaaccacatgagtctccaaggcttctcattaaagtcacac
ctggcctcatcacgttcggagctgggaccaagctggagctgaaacggact
gatgctgcaccaactgtatccaatcttcccaccatccagtgaacagttaa
catctggaggtgcctcagtcgtgtgcttcttgaacaacttctaccccaaa
gacatcaantgtcaaagtgggaagwtttgatgggcagatgaaaccgaaca
aaaatggcgtccctggaacagtttgggaactgatcagggacaggcaaaga
acagcacctacagcatggaagcagcaacccttcaacgtttgaccaaggac
cgaagttattgaaaccgaccataacagctt
14B: end of coding strand, obtained by sequencing
opposite strand, deducing complement, and
arranging in 5′-3′ order (SEQ ID NO: 16)
nngnnccctcacgttgaccaagggnngagtatgaaggacataacagctat
acctgtgaggccactcgcaaggcatcaacttcacccattgtcaagaggtt
caacaggaatgagtgttagagacaaaggtcctgagacgccaccaccagct
ccccagccccatcctattttcccttttaaggtcttggaggttccccccaa
gcgacctaccactgttggggtgctccaaacctcctccccacctccttttc
ctcctcctccctttccttggcttttatcatgctaatatttgcagaaaata
ttccaataaagtgagtctttgcccttaaaaaaaaaaaaaaaaaaaaaaaa
aaaaaa

TABLE 15

Sequence of clone F27 (SEQ ID NO:17, related to a
105 kDa heat shock protein

acaaattggatgcaaaatccaaaattcgagccctccttcgtctccatcag
gagtgtgaaaagttgaaaaagctcatgagttctaacagcacggacctgcc
gctgaacatcgagtgctttatgaatgacaaggatgtctctgggangatga
acaggtcacagtttgaagaactgtgtgctgagctcctgcaaaaaatagag
gtcccccttcactcgttgatggcacagactcagctcaaggctgaanatgt
gagtgccattganatagtgggaggtgcccaagaatcccanctgtnaaaga
aagaattgccaanttctttggaaaagatgtnancacccacnctnaatgnt
gacnaagctgtaggccngnaggctgangcactgnctngtncaatnctttc
tccggcattttaaagttacagantcntnttttcncnccatgctacccntt
ntccnaatatcanctggtcttggaacccnntgnctcnnnangaaaacntt
aacnntgttcnccnaccttntntcatnncnggaaccntgnncgntnnttt
tnttccanacgtgtcnnnncctttnctnngcanaggggnggccctttngt
nctacnaacnnttttcttcttnaccncncnanggnantncnngtnnntaa
ancaanacnannnnnccnnatannactgccnnaacntttntaccntcnan
ccgntnnttttcccnnncnctanngtnanancctcnaannnncntngtnt
nttcatcntncnncnccntnncttgcnnnnangnccnnncacnnccannn
nactnctcttcctannctcn

TABLE 16

Sequence of clone G38 (SEQ ID NO:18, related to an
immunoglobulin protein

acgcggggnattgtcattgcagtcaggnactcagcatggnacatgagggc
tcctgcacagnatttttggcttcttgttgctcttgtttccaggtaccaga
tgtgacatccagatgacccagtctccatcctccttatctgcctctctggg
agaaagagtcagtctcacttgtcgggcaagtcaggatattggtagtagct
taaactggcttcagcaggaaccagatggaactattaaacgcctgatctac
gccacatccagtttagattctggtgtccccaagaggttcagtggcagtag
gtctgggtcagattattctctcaccatcagcagccttgagtctgaagatt
ttgtagactattactgtctacaatatgctacttctcctccgacgttcggt
gggggcaccaagctggaaatcaaacgggctgatgctgcaccaactgtatc
catcttcccaccatccagtgagcagttaacatctggaggtgcctcagtcg
tgtgcttcttgaacaacttctaccccaaagacatcaatgtcaagtggaag
attgatggcagtgaacgacaaaatgggcgtcctgaacagttggactgatc
aggacagcaaaanacngcacctacagcatgancagcacccttacgttgac
caaggacnagtttgaacgacntancnctttacctgngaggccctnacaaa
aatnacttncccattgnnagacttcacaggaatgagtgtanaaacaaggn
ctgnacnccccccnntnccanttcntctatttcctttaaggttggaggtt
ccccannacnccnttntgggggctcaaactntnccnctcttt

TABLE 17
Sequence of clone B3

17A: beginnina of coding strand (SEQ ID NO: 19)

acttgaactggccttcttatccaggataagtggagttcatgcaaggtgga
tccctcatgataggattagtggctttataagaacagtgattgagaaagaa
ttcattctgttgacgcccatgcaagcggaacagcaaactgcttaagaact
catggaatgtggcccactggttagcaccatctcaatgggtgcgccttgga
gaggcactacatgtcctgaggatcacatgctgctgtagagcgtcactctg
cttgttacccttgtggtggtcatcatgtctccccctaatctatgagctng
tatgaaaactctaagagggtttccagaccctcacagggcagtgtcattgg
catttacaatagttgttcttgatctttctcaagcattactgctgggatta
gattaatgaattcagtttaccattcaantaaagttagggttaaggtattt
ttgctaagtnggtttntaatcgtaccaaaatcttggaggagcaatggtca
aaatttttagaaaggccntttangaaaagttttgataacccnantggaaa
ggattnttttggangntcnagggnaancaaagnanttaaccaccaagttn
cnt
17B: end of coding strand, obtained by sequencing
opposite strand, deducing complement, and
arranging in 5′-3′ order (SEQ ID NO: 20)
ctttttaagataaattgaaacctgccagtaagacaacctggggagaaaat
tgccttgttaagttataacaagttctttggttatgaatgcatgtgggttc
ttgggaataatttgtttttaacccataatatgtaaaacatttgatcatgt
gaagctattgaggagcatatgtatctctcttttttaaaaatgtattctca
ttgtaattattcacttaaagaaaagtagaatgtaaccacatcgtggtttt
atgctgcttgaaagaatttcctggtagatatataagctatggaagaaaaa
taaaaattttggagcttgcatcatccgaaaaaaaaaaaaaaaaaaaaaaa
aaagt

TABLE 18
Sequence of clone F39

18A beginning of coding strand (SEQ ID NO: 21):

acgcgggggagccacacaaactcagggaaagctcgaatatggttttcaaa
cctcagatacttggacttatgctttttaggatttcagcctccagaggtga
tattggctacctcaatctccagccaccctgtctgtgcctccaggagatag
cgtcagtctttccttcagggccagccaaaatattagcaacaacctacact
gggttcaaaaaaaccacatgagtctccaaggcttctcattaaagtcacac
ctggcctcatcacgttcggagctgggaccaagctggagctgaaacggact
gatgctgcaccaactgtatccaatcttcccaccatccagtgaacagttaa
catctggaggtgcctcagtcgtgtgcttcttgaacaacttctaccccaaa
gacatcaantgtcaaagtgggaagwtttgatgggcagatgaaaccgaaca
aaaatggcgtccctggaacagtttgggaactgatcagggacaggcaaaga
acagcacctacagcatggaagcagcaacccttcaacgtttgaccaaggac
cgaagttattgaaaccgaccataacagctt
18B END OF CODING STRAND, obtained by sequencing
opposite strand, deducing complement, and
arranging in 5′-3′ order (SEQ ID NO:22):
nngnnccctcacgttgaccaagggnngagtatgaaggacataacagctat
acctgtgaggccactcgcaaggcatcaacttcacccattgtcaagaggtt
caacaggaatgagtgttagagacaaaggtcctgagacgccaccaccagct
ccccagccccatcctattttcccttttaaggtcttggaggttccccccaa
gcgacctaccactgttggggtgctccaaacctcctccccacctccttttc
ctcctcctccctttccttggcttttatcatgctaatatttgcagaaaata
ttccaataaagtgagtctttgcccttaaaaaaaaaaaaaaaaaaaaaaaa
aaaaaa

TABLE 19
Sequence of F2

(SEQ ID NO:23)
acctccccaaaatgactcttttggagcacagttacctccaatgcatcagc
agcaaagcagatctgtaatgacagaagaatacaaagttccagatggaatg
gttggatttataattggcagaggaggtnaacagatctcacngngngcagc
aggaatctggatgcangatacanatagcncctgatagtggtggcctacca
naaaggtctngtatgctaactggaacacctgaatctgtccagtcagcaaa
aagattattggaccagattgttgaaaagggaagaccagcccctggccttc
atcatggngatggacctggaaatgcagttcaggaaatcatgattccancc
agcnaagcnaggactagnttattcggaaaggggggccgagactattaatc
nnctttcncgaacggcctgggtgttaaaatggttatgattnaagatnggc
ctcaaaaaccntgggggtgatnaacctcttagggattnggggttnnccat
acaaagttnagcaagccaagcnnnangtattaggagttaattngtgaana
aggtggtttcagagaagtgcggaatgagtatggctcaanaataggaggca
atgaagggatagatgtcccaatttccaagatttgctgttggcattgtaat
aggaagttntttnaaaaanaacnnccaaaaaaaaaaa

TABLE 20
Sequence of F21

20A beginning of coding strand (SEQ ID NO: 24):

ttnngnantacngngcncaacccnttttaggaacggnccccantgtgctg
gaattcgcccttagcttggtcgcggccgaggnnctacggctcaaaacccc
gtcaccacggcccggatcgggaagtgtcaagtgggagccgggttcccccg
catttgcagccgccacanctgaacccggacccctcaccccagccgggcnc
taccgccntttttaacntgtnctnnngngggngggatcctggagttngnt
ttgataaaaaaanccccccctgtgccngngggnagnaccnncnntntncc
tccaaaccnngnctgctnccaataaaananaggncntcgtggnnttntat
acatacaaaancnccctnatnatcngnaacnacnaccnnnntncctnnct
ngttcnantancccncancngacaaatcnccctntncacatncccccncc
naacnnnctacaantcnacaaacccctnncnacccccattnatcttagcc
cncccntaggncnancntacaccnanccaatcnngcacancnaanacccn
gccnccccctccnctacnaacctccnnacctntcatacnttccnnccgtc
ncccnntnatcaacnnctccnntacnncnctcccccccccnannngttcc
cccaaancnnnccactaancacccgnacctttnnnncccntnaanagncc
atcccctaantncccaccccctttcnctcnntnactntntnnnnnccccc
ncacacancantntacnnntnttctntncaccacaaccatttccccnant
ttnacntccaatctcantcctacccccnncnacacccncacctcacttca
accatanaccnnncncnttcgtccccancncaccncannncccctnncnc
acncactcttcnnntcnntncnnnttcacccantaccccntcgcaacgnt
cccncnantnccannntcccncacn
20B end of coding strand, obtained.by sequencing
opposite strand, deducing complement, and
arranging in 5′-3′ order
(SEQ ID NO: 25):
gaagngnaaaattttttttaaaaaaaaaaaaaaaaaaaaaaa

TABLE 21
Sequence of F38

91A beginning of coding strand (SEQ ID NO: 26):

gntctgtgaaanganatagannggctcgaaggaccaaggcancttcctga
ctgaagaccagatcctgccgctgttgctgggtatcagcagaggccttgag
gctattcatgccaaaggttatgcacacagggacctgaagcccgtngnnna
ttttgcttggtgatgaggggcanccaantntaatggacatgggttntntt
aatcanttcatgcattcaagtggagggctctcgccaggcactaactcttc
aggactgggcagctcagcggtgcaccatctcctaccgggcacctgaactt
ttttctgtgcaaagcctn
21B end of coding strand,obtained by sequencing
opposite strand, deducing complement, and
arranging in 5′-3′ order
(SEQ ID NO:27):
ctagnggcctttgtttttnntnaggtttcccggggggtggcncccttttt
ntttntngggccccccttttttgnantggntntttttttncnncgccctc
tgggggggnntcncgcggcgcntttttttgttgngggggntttgggggng
ncnntttcttcgtgggnccgntnttttttttntncnaaaccccnngnttc
nttnttgggggctntttgtgnnncgnngggggnngntttttcccttnttt
ttngggggnggccccncnntattgnngtttnccaanngggggcganngng
cccntcttgggccaaattnagngngcntcccccncannccccccggcttt
ttttancnttgtgnanntttttntttttttnttgtgggggggggcccccc
gcgggggnctncntnccctntncnncncngtnaggntgggggnntttccc
cccccngnttnnggccgnncccccccccnntttnnntnnannnngngggc
nttttggggnangncccntnnaggggntttttncccccnttgggncncnn
nccntttttnccgggnggntttnccngntnnnggcanggnnagggggncc
ntnttttttttccctttttccccccantcnngggnnngggccctnggggg
ggnnggggggaantttanccnnnantttgggncnaanntngggnnggggg
ggcccccnncnttttntttcccntntngggggnnaaaaaaancntnaaaa
ggnnaatnangttaaacccncccaaaaaaaaaaaaaaaaaaaaaaaaaaa
aaaaaaaaaaaaaaaaaaaaaaaaaaaaaaaaaaaaaaaaaaaaaaaaaa
aacaaaaaaa

TABLE 22
SEQUENCE OF F40

(SEQ ID NO: 28)
acgcgaggtttccggcttgaggatccgtcgcgtttctgaggcggcgcctc
agtcggcagttaggatcggctcggagcggtgggtctcggccccggcgtca
ccatgggggccgtcctcggcgtcttctccctcgccagctgggtcccgtgc
ctctgtagtggtgcatcatgtctgctgtgcagttgctgtcccatcagtaa
gaattccactgtaactcgggtcatctacgcttttatcctcttccttggca
ctattgtgtcttgcatcatgatgacagaaggcatacaaactcaactgaag
aagattcctggattctgtgaaggaggatttcaaatcaagatggttgatac
aaaggcagagaaagattgtgacgtgctggtcggttttaaagctgtgtatc
ggatcaactttgctgtggccatttttttctttgccttctttttgctcatg
ttaaaagttaaaacaagtaaagatcccagagcagcagtgcacaacgggtt
ttgggttcttcaaaatcgctgccattatggtatcatgattggatctttct
acatccctggggggagttttactgaagtctggttttttgctggaatgttg
ggggcctctttcttcattatcatccagctggtgctcttggtagacatggc
tcactcttggaatgaattatgggtaaatcgaatggaggaaggaaacccaa
ggctctggtatgctgccttgctgtcctttacaagcctcttttacatcctc
tccatcgtctttgccgcgctgctctacgtcttttacaccaagcctaacga
ctgcacagaaaacaaggtcttcatcagcctcaacctgattttttgtgttg
cagtttcatattgtgtccatcctccgtaaagttcaggaacatcagcctcg
ctctggcctcctgcagtcctccatcattactaaga

TABLE 23
SEQUENCE OF G9

(SEQ ID NO: 29)
acgcggggatctaggaggctgctgacctccaacaggaatttcaccactta
accctctagaagtcccactacttaatacttcagtacttctagcatcaggt
gtttcaattacatgagctcatcatagccttatagaaggtaaacgaaacca
cataaatcaagccctactaattaccattatactaggactttacttcacca
tcctccaagcttcagaatactttgaaacatcattctccatttcagatggt
atctatggttctacattcttcatggctactggattccatggactccatgt
aattattggatcaacattccttattgtttgcctactacgacaactaaaat
ttcacttcacatcaaaacatcacttcggatttgaagccgcagcatgatac
tgacattttgtagacgtaatcttgacttttcctatacgtctccatttatt
gatgaggatcttaaaaaaaaaaaaaaaaaaaaaaaaaaaaaaaaaaaaaa
agaaaaaaaaaaaaaaaaaaaaaaaaaaaaaaaaa

TABLE 24
SEQUENCE OF G16

(SEQ ID NO: 30)
acgcggggnagttgcggccctgtgcgcttccacagcgtcccagcctttgt
cctggcctcagctcctgcttcctgacctgcttgagttccagtcctgactt
cctttggcgataaacagcagtatggaaatacctcacagtggatgttcctg
gccatggaaaggtgctggtggacattgcatatggtggggcattttatgcg
tttgttagtgcagaaaaattaggacttgatgtgtgttctgcgaagacgag
ggaccttgtggatgcggcaagtgcattgacaggagcagtgaaagcacagt
ttaaaatcaaccatcctgagagtgaagaccttggttttttgtatggatcc
atcttgacggatggaaaagatgcttatagtgaggaggccaccaccaacat
ctgcgtgtttgccgatgaacaggttgacagaagccccaccggctcgggag
tgacagccagaattgctctgcagtatcataaggggctcctgcagctgaac
cagaccagagccttcaaaagcagcgcaactggctcggtgttcacaggctg
tgccgtgagggaagcaaagtgtggagatttcaaagctgtcatagtggaag
ttgcaggacaagcccacttacncagggrcagcaaacttgmcagtggaaga
tggtgaccccctaagggatggcttttttttcaagkgacttttttttcata
agttttaaaggntttgcttanaaagaaatwccycttaaatggggntttnt
ctgnattatgggcnaaccngtntccngttacccttaccgggtaattttta
nactttttaaaaagtatgtttagtanactgngtttaaatgtaaggnntta
tacttcctntttgtcacccntttgngcataaatgtngntaatggggcnaa
canagngccaanaatccggaaacttttnaagccttngtntncaaggcnaa
attnaagcccngttncttaatttntgctgnggaaatgataaaacttgtaa
gttttttttgattgntagnttcagctntnanccccccnatagtaaatgtt
gngngtttgttnttttaatgttccngtttgngtaaaataaaggcntcntg
taagnttccaaaaaaaaaaaaaaaaaaaaaaaaaaaaaaaaaaancaaaa
aaaaaaaaaaaaaaaaaaaaa

TABLE 25
SEQUENCE OF G24

(SEQ ID NO:31)
acgcggggagactggggaaatcgaaattatttcagcagctgtcttcagtc
actgggtgtgtcttcctgcacagtggacttgacgggcctggagggaggag
aagaaattgaaacctatctcatcctctagtttatttacatcattggattc
ggttcctgtttggaaacttaactggtcctcatggcgttttcgggcacagg
gacactggtggcttccattgtctccaagatgacatcttcagcagccatgg
tcaaagctggaggggcagtctccggcaccattctagctggttcgaagggg
ctcatcctgttaacacagagtgccctgggctctgcaacatctgcacttgg
agcaccgaaggttggcaccattttatctgggttctctgcctccaccttgg
ctgcttcccccattggagccaaggctgttgtggctgtgcttgggggagcc
atgacagtagctgctgtaccacctgcgctgagtgctgtgggcttcaccgc
ctcaggaattgcagcctcctctctagcagctaagatgatgtccttgtcag
ctattgctaatggggatggagtcccagctggtggcctggtggccattttg
cagtctgctggagctgctggactttccgtgccatctactgtcatcgtggg
ctttgcagggtttgccgttgtggccagtgtcatgaacatctgtgaaagtt
tttacccttttttcatgggatcagaggtggcagacatggcgacaaaggtg
gcagatatggcgataaaggtggcagacatttcaacaaaaaaggcactgcc
caacttgtttaacccagagaaggattaattacccccaggcgacaatttcc
taacacatattgcctaggtggaaataaattaaaccgagtttttaaaaaaa
aaaaaaaaaaaaaaaaaaaaaaaaaaaaaaaa

TABLE 26
SEQUENCE OF G28

(SEQ ID NO: 32)
accaccggnaccacagtncagcaacgtcaagnatggacatcctgtcttac
atgnaggccggcaggcagccctgagcgccagggggaagtaactgatgccc
ccaccctacccctaccccctgcccatcatgcaagagccgaggaggggctc
ttcaggaaggaagccacattcccagtcattctacccccaccccagattct
ctttcttattacataaaagacaagcctggcacaactgtgtgtctgaacca
ctgtggacacgtgacaattgtcccagtgtgtgcatggctacacagccacg
tatctgcctgcttgaaacccagggatggtccatctgtgtttacggcttgg
cacaacaccctcatatttttttcagctttctgttccaaatgagcccaaag
gaaacacaagttctaggtccaatggttctgctcaaacctgaacatcattc
ttggggccagcatctcccacacgcccacactacacaccaccagcctcctt
cttccttcctgaaggaccctcctgagcccccaagcccatcccacagtgct
cctgagaccagccaagacaactgtgagcgcgatggccgtgtaccttgccc
gggcggccgtcgaaagggcgaattccagccacttggcggccgtactagtg
gatccgagctcggtccaacttgatgcatacttggantnttntatagtggc
acctaaatagcttggngtaatntgggnatagctgnttctgngngaaatgg
tnttccgttanantccnacaacnacaacccgnacataagtgtaaanctgg
ggngcnangangncnnactanttantnnntngnctnntgccnttcannng
naactnnnncnntnntnnantgcaccccggnnggngntnn

TABLE 27
SEQUENCE OF B46

(SEQ ID NO:33)
accacggggacaggatttgggacgttcctgatgaccgtgttgctgaaata
cctgtgcgcagaatatggctggcggaatgctatgttcatccaaggtgctc
tgtccctgaacctgtgtgtctgtggggcgctcatgaggcccctctctccc
gagaagttagaaaactgcccagaagcggaagagccgtgtgctctcccagc
ttactccactgaatctgttaagtctgggggaccactgggcatggctgaag
aacaggacagaaggcccgggaatgaggagatggtgtgtgaccttcaaaca
caggagtgtcaaggtcaaacccatccgaggaagaatgtgtgtgccttccg
ggttctgaagacagtgagccagctcactgtgcaagtccggaggggcttta
gggactggcactcaggctattttgggacggcttcactcttcaccaaccgc
atgtttgtagcctttattttctgggctctgttcgcatacagcagctttgt
catccctttcatccacctgcccgaaatcgtcagtttgtataacttgtcng
aaccaaaatgacacattccctcttgacctcgattatagcaatacttcaca
tcttcgggaaggtgatctttgggggccgtggnccacctcccttgtatcan
cgtctggaatggtcttcctnatnngttacttnaccctcgtcctnnnatnt
tnctttttgcctttgatgcncacctccccaanntggntnnctttgggccc
taatgggntttctagcggttttntcctnnncctggggnacnangactgnt
ngcntnnaccttngcnatgctacgnntntattnnccanggntttnncttn
tngancccntnnngaggatttncnccca

TABLE 28
SEQUENCE OF F16

(SEQ ID NO:34)
acttgctggtggtccatccctgcactatttccaagccaggtcttgggatc
agtgacttgcttcctgcagtgctgtggtcacaggcccctccagtcagcca
tgtctagcagcttggcaggcttaggggctcggtgtgcagatttctttttg
tgcagcagaatgccaagagactgagcagagagtcttcttaaaacctaccc
taagggccgccaggtccatgtgcccgtggtaggtctggggccaggcagct
gctgctgtcctgatcccccctcccttagctatggatccttgtagtagagg
tcagccgctcctggcgcaaacaggatccagtagctgtctgccacccacga
ggtttctaagataccatgtggagacagggtcctccccatccttttcagaa
atagcagcttgttttctctgctggcatgtgtccagggcaccaggtcccag
cagggggaggcagactgttaactgaacscgcataggaacaggaggcagcg
agagcctgtgggaggaagggccttctagtgaccctgcttctccatcctgc
ctccctcctgtccacccagtttcagagttgggaggggaggagccatggct
ccctcacccaggcttggtggctgtggccctgttttagagccatggcagca
caggctggtgagtggtagagttgt

In the above Tables, “N” denotes “unknown”.

Master Table: Identification of Mouse and Human Database Proteins (BLASTX) and
Nucleic Acids (BLASTN) corresponding to indicated clones

| | | | Mouse Database | | Human Database | |
|---|---|---|---|---|---|---|
| | Related Database | | Seq ID | | Seq ID | |
| Clone | Protein | Ef | BLAST N | BLAST X | BLAST N | BLAST X |
| A8 | Ig kappa chain | U | M21796 | S01320 | S65921 | AAB28160 |
| | precursor | | | and many | | |
| | | | | others | | |
| A34 | Immunoglobulin mu | U | X03690 | A24976 | AF144029 | AAH01872 |
| | chain C region | | | | | |
| A39 | Ig heavy chain | U | BC010327 | AAH10327 | S65761 | AAB28159 |
| | variable region | | | AAA51630 | | |
| | gamma-2b C-region | | | | | |
| A48 | Immunoglobulin | U | BC008237 | AAB59665 | S65761 | AAB28159 |
| | heavy chain | | BC002121 | | | |
| | constant region | | L35252 | | | |
| B3 | Unknown; Mus | F | BC021774 | — | AL139806? | — |
| | musculus, clone | | | | | |
| | IMAGE: 4238277 | | | | | |
| B45 | Kidney androgen | F | NM_010594 | NP_034724 | AF319957 | P15267 |
| | regulated protein | | | | | |
| B46 | NAS˜hypothetical | F | AK003423 | BAB22782 | XM_087215 | XP_087215 |
| | protein˜putative | | | | AC093171 | |
| C22 | Disabled-2 p96 | U | BC016887 | Noncoding | AC008846 | Noncoding |
| | | | AF60579S2 | region | AF218839S2 | region |
| | | | | AAG44669 | | AAH03064 |
| | | | | | | P98082 |
| | | | | | | AAF23161 |
| E39 | monoclonal antibody | U | U65535 | AAC04542 | S65921 | 1FH5L |
| | kappa light chain | | | | | |
| F1 | SON protein | F | NM_019973 | NP_064357 | NM_058183 | NP_003094 |
| | | | AF193597 | AAF23121 | and others | AAK07692 |
| | | | AF193607 | | | |
| F2 | Far upstream | F | NP_476513 | NP_476513 | NM_003902 | NP_003893 |
| | element (FUSE) | | AAH14763 | | U05040 | AAA17976 |
| | binding protein 1 | | | | BC010083 | A53184 |
| | | | | | | AAH17247 |
| F4 | Kidney androgen | F | NM_010594 | NP_034724 | AF319957 | AAH08576 |
| F6 | regulated protein | | | | | AAG50272 |
| F5 | Claudin 10 | F | AK020131 | BAB32005 | NM_006984 | NP_008915 |
| | | | NM_021386 | NP_067361 | U89916 | BAB71030 |
| | | | | NP_076367 | | |
| | | | | XP_127876 | | |
| F16 | Unknown; Mouse DNA | F | AL589870 | NP_044109 | — | — |
| | sequence from clone | | | (not mouse) | | |
| | RP23-118A2 on | | | | | |
| | chromosome 2, | | | | | |
| | complete sequence | | | | | |
| F21 | Unknown | U | AK008415 | — | | AAA59875 |
| F27 | Heat shock protein | F | NM_013559 | BAA11035 | XM_036358* | XP_036357* |
| | 105 kD | | | | | |
| F38 | Palmitylated | U | NM_011494 | AAD02811 | NP_035624 | NP_003682 |
| | serine/threonine | | | BAA89662 | | CAA06700 |
| | kinase | | | NP_035624 | | |
| F39 | Phosphotriesterase | F | NM_008961 | NP_032987 | XM_050904 | NP_109589 |
| | related protein | | | | | |
| F40 | Tumor | U | BC011295 | AAH11295 | AF112227 | AAD22448 |
| | differentially | | | AAH22901 | | AAB48858 |
| | expressed 1 | | | NP_036162 | | AAD54420 |
| | | | | AAD54420 | | NP_006802 |
| | | | | | | AAB48858 |
| | | | | | | AAD34641 |
| G9 | Cytochrome oxidase | U | V00711 | CAA24090 | AF381988 | AAK17824 |
| | III | | J01420 | | | AAL54598 |
| G16 | Mus musculus 16 | F | AK018449 | BAB31217 | XM_053376 | XP_053376 |
| | days embryo lung | | | AAH04753 | | AAH12131 |
| | cDNA, RIKEN | | | | | |
| | full-length | | | | | |
| | enriched | | | | | |
| | library, clone: 84304 | | | | | |
| | 28P19: hypothetical | | | | | |
| | protein, full | | | | | |
| | insert sequence | | | | | |
| G24 | Homolog to ERG2 | | AK003665 | BAB22924 | AC068491? | AAH22800 |
| | protein; TLH39 | | | | | NP_114425 |
| | protein precursor | | | | | AAG35730 |
| G26 | Hydroxysteroid | U | BC013449 | NP_032320 | NM_000862 | NP_000853 |
| | dehydrogenase-4, | | NM_008294 | | AL121995 | AAA51831 |
| | delta<5>-3-beta | | AB049424 | | S45679 | |
| | | | L16919 | | X53321 | |
| | | | | | X55997 | |
| | | | | | M38180 | |
| | | | | | M63397 | |
| | | | | | M35493 | |
| | | | | | M27137 | |
| | | | | | M28392 | |
| G28 | Glutathione | U | BC003339 | BAB21943 | Y11107 | XP_087620* |
| | peroxidase 3 | | AK004942 | BAB23686 | | |
| | | | AK002262 | | | |
| | | | AK002219 | | | |
| G38 | Igg2a Fab fragment; | F | AF178454 | 1310829 | S65921 | AAB28160 |
| | anti-human | | | | | |
| | apolipoprotein A | | | | | |
| | mAb(a) 20L kappa | | | | | |
| | light chain | | | | | |
| H1 | Ubiquitin protein | F | BC007184 | Noncoding | XM_046129 | Noncoding |
| | ligase Nedd-4 | | U96635 | region | | region |
| | | | | AAB63360 | | P46934 |
| | | | | | | BAA07655 |
| H8 | ATPase, H+ | F | BF539 | NP_0385 | AC0231 | XP_08836 |
| | transporting, | | | | | |
| | lysosomal; | | | | | |
| | Ac39/physophilin | | | | | |

“Ef” stands for effect and can be favorable(“F”) or unfavorable (“U”).
“?” (see B3 and G24) indicates questionable similarity (or rather significance) due to very short region of similarity
“—” in the BLAST X column could indicate clone is in untranslated region
*indicates removed at submitter's request, yet still there
These sequences may be accessed at the homepage of the National Center for Biotechnology Information (http://www.ncbi.nlm.nih.gov/) by setting the search dropdown box to “protein” or “nucleotide” as appropriate, entering the database ID number in the text box, and clicking “GO”.

Comments on Clones and Cognate Database Sequences
In these comment, “Full-length” means that it includes complete coding sequence
B3 clone is not full-length. It is similar to a DNA sequence in the database but does not match any protein in the database (no protein is given for the DNA sequence).
B45 clone is full-length as are the mouse and human homologs. B45 is an exact match to the database proteins whereas F4 and F6 have a one amino acid difference.
B46 clone is possibly full-length.
BAB22782 hypothetical protein˜putative [Mus musculus] is full-length.
XP_{—}087215 similar to unnamed protein product [Homo sapiens) is full-length.
BAB70909 unnamed protein product [Homo sapiens] is full-length.
   C22 clone is not full-length. Similarity is to 3′ non-coding region of AF260579S2 Mus musculus disabled-2 p96 (Dab2) gene, exons 2-15 that encodes protein AAG44669 disabled-2 p96 [Mus musculus] (full-length). This protein is similar to AAH03064 disabled (Drosophila) homolog 2 (mitogen-responsive phosphoprotein) [Homo sapiens], P98082 Disabled homolog 2 (Differentially expressed protein 2) (DOC-2) [Homo sapiens], and AAF23161 disabled-2 [Homo sapiens] (all full-length).
   F1 clone is not full-length.
AAF23120 SON protein [Mus musculus] is full-length.
NP_{—}003094 SON DNA binding protein; SON DNA-binding protein; SON DNA-binding protein, KIAA1019; NRE-binding protein [Homo sapiens] are full-length.
AAK07692 NREBP (negative regulatory element-binding protein) [Homo sapiens] is full-length.
   F2 clone is not full-length.
NP-476513 DNA segment, Chr 3, ERATO Doi 330, expressed; far upstream element (FUSE) binding protein 1 [Mus musculus] is full-length.
AAH14763 Similar to far upstream element (FUSE) binding protein 1 [Mus musculus] is full-length.
NP_{—}003893 far upstream element-binding protein; far upstream element binding protein; FUSE-binding protein [Homo sapiens] is full-length.
AAA17976 FUSE binding protein (Homo sapiens] is full-length.
A53184 myc far upstream element-binding protein—human—is full-length.
AAH17247 far upstream element (FUSE) binding protein 1 [Homo sapiens] is full-length.
   F4 and F6 clones are full-length.
NP 034724 kidney androgen regulated protein [Mus musculus] is full-length.
Several other mouse matches are full-length.
AAH08576 kidney androgen regulated protein. [Homo sapiens] is full-length.
AAG50272 FKSG22 [Homo sapiens] is full-length.
   F5 clone is possibly full-length.
NP 076367 claudin 10; claudin-10 [Mus musculus] is full-length.
BAB32005 and XP_{—}127876 putative—similar to CLAUDIN-10 [Mus musculus] are full-length.
BAB71030 unnamed protein product [Homo sapiens] is full-length.
Note 1: Although the human protein is “unnamed”, it is very similar to the mouse Claudin-10 listed above.
Note 2: The first 73 amino acids of these Claudin-10 proteins (including Clone F5) differ significantly from other proteins that are also named Claudin-10.
   F16 clone encodes an unknown protein. The only significant similarity is to a genomic nucleotide sequence of Mus musculus. It is not known if F16 is full-length.
   F21 clone encodes an unknown protein. A small portion of nucleotide sequence is similar to a mouse clone encoding an unknown protein. It is not known if F21 is full-length.
   F27 clone is probably not full-length. Only a portion of its protein sequence is similar to Mus musculus and Homo sapiens Heat shock 105 kD protein (both of which are full-length). The rest of the F27 sequence is novel.
   F38 clone is possibly full-length.
AAD02811 palmitylated serine/threonine kinase [Mus musculus] is probably full-length.
BAA89662 F5-2 [Mus musculus] is probably full-length.
NP_{—}035624 and others serine/threonine kinase 16 [Mus musculus] is probably full-length.
NP_{—}003682; and others serine/threonine kinase 16; protein expressed in day 12 fetal liver [Homo sapiens] is full-length.
CAA06700 PKL12 protein [Homo sapiens] is full-length.
   F39 clone is probably not full-length. Only a portion of its protein sequence is similar to Mus musculus and Homo sapiens Phosphotriesterase Related Protein (both of which are full-length). The rest of the F39 sequence is novel.
   F40 clone is not full-length.
AAH11295 Similar to tumor differentially expressed 1 [Mus musculus] is full-length.
AAH22901 Unknown [Mus musculus] is full-length.
NP_{—}036162 tumor differentially expressed 1 [Mus musculus] is full-length.
AAD54420 membrane protein TMS-1 [Mus musculus] is full-length.
AAB48858 Diff33 gene product [Homo sapiens] is full-length.
AAD34641 transmembrane protein SBBI99 [Homo sapiens] is full-length.
NP_{—}006802 tumor differentially expressed 1; placental transmembane protein [Homo sapiens] is full-length.
   G9 clone is not full-length.
CAA24090 cytochrome oxidase III [Mus musculus] is full-length.
AAK17824 and AAL54598 cytochrome c oxidase subunit III [Homo sapiens] are full-length.
   G16 clone is not full-length.
BAB31217 evidence:NAS˜hypothetical protein˜putative [Mus musculus] is full-length.
AAH04753 similar to RIKEN cDNA 2810055F11 gene [Mus musculus] is full-length.
AAH12131 similar to RIKEN cDNA 2810055F11 gene [Homo sapiens] is full-length.
   G24 clone is full-length.
BAB22924 homolog to ERG2 protein˜putative [Mus musculus] is full-length.
AAH22800 Unknown [Homo sapiens] is not full-length.
NP_{—}114425 and AAG35730 TLH29 protein precursor [Homo sapiens] appear to be full-length.
   G26 clone is not full-length.
NP_{—}032320 and others hydroxysteroid dehydrogenase-4, delta<5>-3-beta; 3-beta-hydroxysteroid dehydrogenase/delta-5-delta-4 isomerase [Mus musculus] are full-length.
NP_{—}000853 hydroxy-delta-5-stero-id dehydrogenase, 3 beta- and steroid delta-isomerase 1; Hydroxy-delta-5-steroid dehydrogenase, 3 beta- and steroid [Homo sapiens] is full-length.
   G28 clone is probably not full-length.
BAB21943 and BAB23686 glutathione peroxidase 3˜putative [Mus musculus] are probably not full-length.
XP_{—}087620 similar to glutathione peroxidase 3 (plasma) [Homo sapiens] may be full-length.
   H1 clone is not full-length. Similarity is to 3′ non-coding region of U96635 Mus musculus ubiquitin protein ligase Nedd-4 mRNA that encodes protein AAB63360 NEDD-4 [Mus musculus] (full-length). This protein is similar to P46934 NEDD-4 protein [Homo sapiens] and BAA07655 KIAA0093 gene product is related to NEDD-4 protein. [Homo sapiens] (full-length).
   H8 clone is nearly full-length.
NP_{—}038505⁻ ATPase, H+ transporting, lysosomal 38 kDa, V0 subunit D isoform 1; ATPase, H+ transporting, lysosomal (vacuolar proton pump), 42 kDa [Mus musculus] is full-length.
XP_{—}088368 similar to Ac39/physophilin [Homo sapiens] is full-length.

1. A method of protecting a human subject from kidney damage which comprises administering to the subject a protective amount of an agent which is
(1) a polypeptide which is substantially structurally identical or conservatively identical in sequence to a reference protein selected from the group consisting of (androgen related protein) NP_{—}034724 and P15267;
(SON protein) NP_{—}064357, AAF23121, NP_{—}003094, and AAK07692;
(FUSE binding protein 1) NP_{—}476513, NP_{—}003893, and AAA17976;
(claudin 10) BAB32005, NP_{—}067361, NP_{—}076367, XP_{—}127876, NP_{—}008915, and BAB710301;
(heat shock protein) BAA11035 and XP_{—}036357;
(phosphotriesterase related protein) NP_{—}032987 and NP_{—}109589;
(ubiquitin protein ligase Nedd-4) AAB63360, P46934, and BAA07655; and
(Ac39/physophilin) NP_{—}0385 and XP_{—}08836; or
(2) an expression vector encoding the polypeptide of (1) above and expressible in a human cell, under conditions conducive to expression of the polypeptide of (1);
where said agent protects said subject from kidney damage.

2. A method of protecting a human subject from kidney damage which comprises administering to the subject a protective amount of an agent which is
(1) an antagonist of a polypeptide, occurring in said subject, which is substantially structurally identical or conservatively identical in sequence to a reference protein selected from the group consisting of
(disabled-2 p96) AAG44669, AAH03064, P98082, and AAF23161;
(palmitylated serine/threonine kinase) AAD02811, BAA89662, NP_{—}035624, NP_{—}003682, and CAA06700;
(tumor differentially expressed 1 protein) AAH11295, AAH22901, NO_{—}036162, AAD54420, AAD22448, AAB48858, AAD54420, NP_{—}006802, AAB48858, and AAD34641;
(cytochrome oxidase III) CAA24090, AAK17824, and AAL54598;
(TLH39 protein precursor) BAB22924, AAH22800, NP_{—}114425, and AAG335730;
(hydroxysteroid dehydrogenase 4 delta <5>-3 beta) NP_{—}032320, NP_{—}000853, and AAA51831; and
(glutathione peroxidase III) BAB21943, BAB23686, and XP_{—}087620; or
(2) an anti-sense vector which inhibits expression of said polypeptide in said subject,
where said agent protects said subject from kidney damage mediated by said polypeptide.

3. A method of protecting a human subject from kidney damage which comprises administering to the subject a protective amount of an agent which
(1)
(a) down-regulates expression of an “unfavorable” protein which is identifiable as a homologue in said subject of a mouse marker gene which is
(i) up-regulated in a first group of mice, which are experiencing or are prone to kidney damage, and/or
(ii) down-regulated in a second group of mice, which are protected against or otherwise less prone to kidney damage, relative to the other group, or
(b) is an antagonist for the expression product of the “unfavorable” gene, or
(c) degrades that product, or
(2)
(a) up-regulates expression of a “favorable” protein which is identifiable as a homologue in said subject of a mouse marker gene which is
(i) down-regulated in said first group of mice and/or
(ii) up-regulated in said second group of mice, relative to the other group or
(b) is an agonist for the favorable protein, or
(c) inhibits the degradation of the favorable protein, or
(d) is said favorable protein or a protein which is substantially or conservatively identical thereto, or
(e) is an expression vector comprising a DNA sequence encoding said protein (d) and operably linked to a promoter whereby said protein (d) is expressed in cells of said subject which are transformed by said vector,
where said agent protects said subject from kidney damage.

4. A method of screening for human subjects who have developed, or are prone to development of, kidney damage, which comprises assaying tissue or body fluid samples from said subjects to determine the level of expression of a “favorable” human marker gene, said human marker gene being substantially structurally identical or conservatively identical in sequence to a protein selected from the group consisting of
(androgen related protein) NP_{—}034724 and P15267;
(SON protein) NP-064357, AAF23121, NP_{—}003094, and AAK07692;
(FUSE binding protein 1) NP_{—}476513, NP_{—}003893, and AAA17976;
(claudin 10) BAB32005, NP_{—}067361, NP-076367, XP_{—}127876, NP_{—}008915, and BAB710301;
(heat shock protein) BAA11035 and XP_{—}036357;
(phosphotriesterase related protein) NP_{—}032987 and NP_{—}109589;
(ubiquitin protein ligase Nedd-4) AAB63360, P46934, and BAA07655; and
(Ac39/physophilin) NP_{—}0385 and XP_{—}08836;
and directly correlating the level of expression of said marker gene with the development of kidney damage in said patient.

5. A method of screening for human subjects who have developed, or are prone to development of, kidney damage, which comprises assaying tissue or body fluid samples from said subjects to determine the level of expression of an “unfavorable” human marker gene, said human marker gene being substantially structurally identical or conservatively identical in sequence to a protein selected from the group consisting of
(disabled-2 p96) AAG44669, AAH03064, P98082, and AAF23161;
(palmitylated serine/threonine kinase) AAD02811, BAA89662, NP_{—}035624, NP_{—}003682, and CAA06700;
(tumor differentially expressed 1 protein) AAH11295, AAH22901, NO_{—}036162, AAD54420, AAD22448, AAB48858, AAD54420, NP_{—}006802, AAB48858, and AAD34641;
(cytochrome oxidase III) CAA24090, AAK17824, and AAL54598;
(TLH39 protein precursor) BAB22924, AAH22800, NP-114425, and AAG335730;
(hydroxysteroid dehydrogenase 4 delta <5>-3 beta) NP_{—}032320, NP_{—}000853, and AAA51831; and
(glutathione peroxidase III) BAB21943, BAB23686, and XP_{—}087620;
and inversely correlating the level of expression of said marker gene with the development of kidney damage in said patient.

6. A method of screening for human subjects who have developed, or are prone to development of, kidney damage, which comprises
assaying tissue or body fluid samples from said subjects to determine the level of expression of at least one human marker protein, where said human marker protein is identifiable as a homologue of a mouse marker gene which is expressed at different levels in a first group of mice who are experiencing or are prone to kidney damage and in a second group of mice protected against or otherwise less prone to kidney damage, and
correlating said level of expression of said human marker gene with the development of kidney damage in said subject.

7. The method of claim 4 in which the marker gene is one whose expression is down-regulated in the first group of mice.

8. The method of claim 4 in which the marker gene is one whose expression is up-regulated in the second group of mice and/or down-regulated in the first group of mice.

9. The method of claim 4 in which the first group of mice are treated with streptozotocin.

10. The method of claim 9 in which the second group of mice are treated with streptozotocin, but are genetically modified mice protected from diabetes-induced kidney damage by virtue of their genetic modification.

11. The method of claim 10 in which the genetically modified mice are transgenic mice which express a growth hormone (GH) mutant which is a GH receptor antagonist which antagonizes a growth hormone endogenously expressed by said mice.

12. The method of claim 10 in which the genetically modified mice are mice which do not express a functional growth hormone receptor binding protein (GHRBP).

13. The method of claim 9 in which the second group of mice are nontransgenic mice which are not treated with streptozotocin.

14. The method of claim 4 in which the level of expression of the marker protein is ascertained by measuring the level of the corresponding messenger RNA.

15. The method of claim 4 in which the level of expression is ascertained by measuring the level of a protein encoded by said marker gene.

16. The method of claim 1 in which the subjects are diabetic and the kidney damage is, at least in part, diabetes-induced.

17. The method of claim 1 in which the subjects are hyperinsulinemic.

18. The method of claim 4 in which the first group of mice exhibit kidney damage.

19. The method of claim 4 in which the first group of mice are genetically modified to overproduce growth hormone.

20. The method of claim 18 in which the first group of mice produce a heterologous growth hormone in addition to mouse growth hormone.

21. The method of claim 18 in which the second group of mice are normal mice.

22. The method of claim 1 in which the agent is a DNA, or is encodable by a DNA, which specifically hybridizes to the recited DNA strand of any of SEQ ID NOs: 1-34, or to the complementary strand thereof.

23. The method of claim 1 in which said polypeptide is at least 80% identical or at least highly conservatively identical to said reference protein.

24. The method of claim 1 or 24 in which said polypeptide is at least 90% identical to said reference protein.

25. The method of claim 1 in which said polypeptide is identical to said reference protein.

26. The method of claim 4 in which said homologue is identifiable by a BLASTN or BLASTX search conducted, using any of SEQ ID NOS:1-34 as a query sequence, on the NCBI Entrez sequence database(s), on or before the filing date of the instant application, and the E value calculated by BLASTX or BLASTN for the alignment of that homologue, or cDNA encoding that homologue, to the query sequence is less than e-10.

27. The method of claim 26 in which the E value calculated by BLASTN or BLASTX would be less than e-15, more preferably less than e-20, still more preferably less than e-40, even more preferably less than e-60, considerably more preferably less than e-80, and most preferably less than e-100.


SEQUENCE LISTING (34 sequences)

SEQ ID NO: 1 - DNA, 747 bp, Mus musculus
  misc_feature (14)..(14): Unknown
actcagctcctacnggntgcttgtgtggaagccaacgtgccaacattcatctacagcagctcagtgcttgtggctggaccaaattcctacaaggagatcatcctgaatggccatgaggaggagcatcatgaaagcacatggtctaacccatacccatgnnnngtgaaagatggctgagaaggcagtgctggcaacccntgggagcatcctgaaaaatggtggcactttgcatacttgtgccttaagactctctttcatctatggggaagaatgccaagtcacttcaaccactgtgaaaacagcactgaagaacaacagcataattaagaaagatgccacattctccattgccaacccagtgtatgtgggcaatgcancctgggctcacattctggctgccaggagcctacaggaccctaaaaagtccccaagcatccaaggacagttctattacattaacagatgacacccctcaccaaagcnatgatgacttnaaaatgcaccctgagcaaggagtgggggcccncccccttganaccaactgggaagtctttcctctgccccctgctntactnggcnttgccttncctccccnggaaaactggggaaccttcctgctaccnnccannctaccaacttannnaaccccnttttaaaccccccnttggaccnccanngnnaaaaaaangnnntncncccnntcctaccaaaaaggcncaacnnnnaacnnggcnttnancnnnttng

SEQ ID NO: 2 - DNA, 799 bp, Mus musculus
  misc_feature (1)..(1): Unknown
ngggcttncggcccnnananntcccaagcnnccaggnnggtttnttcctttcnangnncccctcaccaaaagtatgntnatttaaaagacccttgancaaagantggggccttngctttgatacanntggagtntttctntggcccnnctncnatnggcntgctttcntcttnaaactntgagnntccnggntacgtccagtnttncaaaataaaancccgcccatttaaccgcctcttgatcacagtgnttaaanagtgatccacctctcctanaagaaggtcagngagatttgggctatgagccaacttgtcagctgggaggnagccaagcagaaaacctcagagtggatcggaaacactagtgatgcagcacagggagattggaaacaaaaagtcncagtgatatgaagagggagaggacatggccctgggtgttattaggtcctccagaaagggacttagaaacaatccaactcttaacaattccattttacactctgtccaacttgtctttgggtcaccagaagccttgcaagtcactggcccagttgcaacccttcagctntaagcacttgtccagcaatgcacaagatgtgcctcagctgcngtgacccaaggatgggtggctgatagtgagttgcctggaacctcttgtaggttagaatttcatcnnggcttccacatctcctttccatgtgccaacgcatttcgtgtctgagaaaattcccatacctttatgaagctcaaagaatagaacacataaaatcttttaatgcgtaaaaaaaatnaaaaaaaaa

SEQ ID NO: 3 - DNA, 875 bp, Mus musculus
acttcaatgtggaccatggctacctggagggcctggttcgaggatgcaaagccagcctcctaactcagcaggactatgtcaacctagtgcagtgtgagacctcggaagacctgaaaattcatctccagaccacggactatggcaacttcctggctaatgaaacaaatcctctcactgtttccaaaattgacacggagatgaggaagaagttctgcagagagtttgactatttccggaatcattccttggagtccctgagcacatttctcacctacatgacatgcagctatatgatagacaatataattctacttatgaatggggccttgcaaaagaaatctgtgaaagaagttctagccaagtgtcacccactgggccgtttcacagagatggaagctgtcaacattgcagagaccccctcagatctcttcaggcttgtgctggttgaaacaccattagctccattctttcaagattgtatgtcttgaaaacacctcttgatgaacttgaatattgaattactgcgcaataaactatacaagtcttaccttgaggcattctacaaattctgcaaggatcacggtgatgtcacagcagacgttatgtgtcccattcttgagtttgaggccgacagacgcgctttaatcatcactctgaactcatttggcactgaactaagcaaagaagacagggagaccctcttccccacctgcggcaggctctatccagaggggttgcggttgttagctcaagctgaagactttgagcagatgaagagagtggcagataattatggagtttacaagcctttgtttgactgctgtcggtggcagtggggggaagacactggaagacgttttctatgagagagaggt

SEQ ID NO: 4 - DNA, 966 bp, Mus musculus
acatacgtgtttacggagttcattatgtttacagattaagcgaatttctgtagttgcatttttatatttttagtatcacattagtataaaaatttgtttaaaatagccaaagagtagttcaatgcataatttgtatgaatttgttaccaagtttcttatgctttctaaaaaatactgtttcctatgaaaattatgtttaatcaaaagtcaagaacccttggggcaaaatgctacaggtggagtcccaccatagtcacctttgaggcacaggaagaggttccatgcatgaatctgcacacatgagcagtgctggctgctgtgcaccgggcccttgggatgaagccatcagcactgacatccagccccgtcttcatcctcaggcttagaaacaagtgggatggtgactttacagtcagtgggaagtaagagcccgcagaataacaaaggcagaaggacaggaaagcgaggtccgagtccgactgtgaggtcatatctggccaaggctccttctagtcagcttacactggtgattatacggaaaatgctcttagatatgagaaaagcaatgtggcatgtaatgagttagtgtaagcttgtgttataaactttccgaatgcttataagtagcctgtgggagctgtaagaaatacttagggccattgatttaagcttgttggctctaacatgactaatacctgtcagcccatggtagggatttcagtgacagtagtggaaacgctttccacagtccacaccactatgttgatcggtgaaagacacccccaggccagtgcatgcttccagagtgtcctgatcagtcagccaggcatggccaccgtccagtgttgatttttatcattagccagctgagcctgcagcacggggctcatttccccgtctggcagatgtggatagctgggcccactcattctgtctctcaaggctatggtaagttcttaaaggggt

SEQ ID NO: 5 - DNA, 1159 bp, Mus musculus
acaagcccgacttgaaagaggcctccagaaaaagtagatgcgtatctgtgcaaacagatcctactgatgaagtgcccaccaaaaagtcaaagaagcataaaaagcacaaaaacaaaaagaagaaaaagaagaaagaaaaagaaaaaaaatataaaagacaaccagaagaatctgagtccaagctgaaatctcatcatgatgggaatctagaatctgattccttcttaaagtttgattctgaaccttcagcagcggcactggaacatcctgtaagagcgtttggcttatctgaggccagtgagaccgccttagtgctggaacctccagtagtctcaatggaggttcaggagtcacacgttctagagactctgaagccagctacaaaagctgcagaactgtcagttgtgtctacatcagtaatctcagagcagtctgagcagccgatgccaggtatgctggaaccatcaatgacaaagatcctggattccttcacagcagcaccagtggcctatgtcaacagcggcgctgaagtcacctgagccccgttgtaacaaatgtcagtggaatatcagaagtctgtgctgaaatctttggagactatgcctccagagacgtcaaagaccacgctggtagagcttcccatagcaaaagtggttgagccatcagaaaccctcacgatagtgtcagagacacctactgaggtgcaccctgaaccaagcccatcaacaatggattttccagagtcatctacaactgacgtgcaaagattgccagagcagcctgtagaagcaccatcggagattgcagattcatccatgacaagacctcaggagtcactggagctgcctaagaccacagcggtggagctgcaggagtcaacggtggcctcagctctggagttgccggggccacctgcgacctccattctggagttgcaggggccccctgtgactccagtgccagagttgcctgggccctctgccaccccagtgccagagttgtcagggcccctttctaccccagtgcctgagttgccagggccccctgcgacagtggtccctgagttgccggggccctctgtgacaccagtgccacagttgtcgcaggaattgccagggcctccagcaccatccatggggttggagccaccacaggaggt

SEQ ID NO: 6 - DNA, 616 bp, Mus musculus
  misc_feature (579)..(579): Unknown
acgcggggtaatttctttctcctgttcactggttgccttaaccctactaaagcatgatgcttttcaaggtcctggtgatcactgtctcctgtggtctgactgtggctttccccctgtcagaattagtttcaatcaataaagaactacagaattcaatcattgacctactaaactcagtctttgaccaactgggatcatacagagggacaaaagctcctctagaggattatacagatgatgatttaagcactgactctgagcagatcatggacttcacgccagccgcaaacaaacagaattctgagttctctactgatgttgagacagtctcctccggctttctggaagaattcactgagaacacagacatcacagtgaaaattccattagctgggaatccagtctcccctacttcctgagaagactccaactttgaaataaacctgcgactgccattcaggatattgtctttgtagaaaatgatataaatctaagatgctcccacaaagaagaaagaacgaggaagtgatcctggatgagaaatgctgtgccaggcatctctaagctctacccttactntgtgtgggtttaagaaataaaccgttcatttgattgc

SEQ ID NO: 7 - DNA, 881 bp, Mus musculus
  misc_feature (9)..(9): Unknown
acgcggggnagtagggaattagaaagattattctaagctatctctaagatgcccttagtgtgatatactttattctaatttcccacacttcaagccatgagattatgaagaattgtcacctaccctcatgggctggctttaacattcgtggaagttttgtccagtgtctgcatgcctaggccaccaaagccttctgtgtggactgtggcagcaggcaaggctgagcgacatgtccagggcacagatctcagctctggtgtgtggtgttggagggtttggtgctctcgtcgctgccaccacatccaacgaatggaaagtgaccacccgagcgtcgtctgtgattaccgccacctgggtttaccagggtctgtggatgaactgcgcaggtaacgctctgggctccttccactgccggccacatttcactatcttcaaagtagaaggttacatccaggcatgtagaggactaatgatcgctgcggtcagcctgggatttttcggttccatttttgcactctttggaatgaaatgtaccaaagtcggaggctcagatcaagccaaagctaaaatccgcttgctttggcccgggattggattcatattgncaggtctggggttccatgacaggctgttcccttgtntgcaaccaaatcncaacngaattctttgatcctntttatatggagcaaaagtttgaattaggggcttgtttttttatcggatgggcngaacttttttntgntcatgggggagnatatttggttttaatatccacacaccaacnccaaaagggtacnntcaanggncccnttngcatgtttttggaccagtttaggngnaaagaaaaaaaaaaaaaaaaaancctngccnancctagggan

SEQ ID NO: 8 - DNA, 611 bp, Mus musculus
aacttctttctcctgttcactggttgccttaaccctactaaagcatgatgcttttcaaggtcctggtgatcactgtctcctgtggtctgactgtggctttccccctgtcagaattagtttcaatcaataaagaactacagaattcaatcatcgacctactaaactcagtctttgaccaactgggatcatacagagggacaaaagctcctctagaggattatacagatgatgatttaagcactgactctgagcagatcatggacttcacgccagccgcaaacaaacagaattctgagttctctactgatgttgagacagtctcctccggctttctggaagaattcactgagaacacagacatcacagtgaaaattccattagctgggaatccagtctcccctacttcctgagaagactccaactttgaaataaacctgcgactgccattcaggatattgtctttgtagaaaatgatataaatctaagatgctcccagcaaagaagaaagaacgaggaagtgatcctggatgagaaatgctgtgccaggcatctctaagctctacccttactctggggtgggtttaagaaataaacgttcattttgaattccc

SEQ ID NO: 9 - DNA, 334 bp, Mus musculus
acgcgggtgagcagcaccctcacattgaccaaggacgagtatgaacgacataacagctatacctgtgaggccactcacaagacatcaacttcacccatcgtcaagagcttcaacaggaatgagtgttagagccaaaggtcctgagacgccaccaccagctccccagctccatcctatcttcccttctaaggtcttggaggcttccccacaagcgacctaccactgttgcggtgctccaaacctcctccccacctccttctcctcctccctttacttggcttttatcatgctaatatttgcagaaaatattcaataaagtgagtctttgcacttg

SEQ ID NO: 10 - DNA, 503 bp, Mus musculus
acgttgtaaaacgacggccagtgaattgtaatacgactcacttatagggcgaattgggccctctagatgcatgctcgagcggccgccagtgtgatggatatctgcagaattcgccctttcgagcggccgcccgggcaggtacagtgtgggtttaccagtggacttgtccacggtcctctcggtcaccaggtgtggcagggcctcgtggcctacaacacaggtataggtctctccggagttccattcctcctctgtcacagtcaggatgctgtgggtaaagtagaagcctggggccccaggctctggcatcggggcactggtcacatacttctcttggggcaagagttgccctccctgaagccactgcacactgatgtctgcaggagagaagcccttcaccaggcaggtgactgtggctgactccctcaggttcagttgctcacgagctggtggcagcaggtacctcggccgcgaccacgctaagggcgaattccagcacactg

SEQ ID NO: 11 - DNA, 482 bp, Mus musculus
acaagatggggactgggacgggcctttgactactggggccaaggcaccactgtcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccakcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgccctccatgcaaggagtgtcacaaatgcccagctcctaacctcgagggtggaccatccgtctttatcttccctccaaatatcaaggatgt

SEQ ID NO: 12 - DNA, 430 bp, Mus musculus
acaccattccacctcccaaggagcagatggccaaggataaagtcagtccgacctgcatgataacaaacttcttccctgaagacattactgtggagtggtagtggaatgggcagccagcggagaactacaagaacactcagcccgtcacggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggtcctacaggactctgacacctacctccacccctccctgtgtaaataaagcacccagcactgccttgggaccctgcaaaaaaaaaaaaaaaaaaaaaaaaaaaaagt

SEQ ID NO: 13 - DNA, 610 bp, Mus musculus
acgcggggaatttctttctcctgttcactggttgccttaaccctactaaagcatgatgcttttcaaggtcctggtgatcactgtcttctgtggtctgactgtggctttccccctgtcagaattagtttcaatcaataaagaactacagaattcaatcattgacctactaaactcagtctttgaccaactgggatcatacagagggacaaaagctcctctagaggattatacagatgatgatttaagcactgactctgagcagatcatggacttcacgccagccgcaaacaaacagaattctgagttctctactgatgttgagacagtctcctccggctttctggaagaattcactgagaacacagacatcacagtgaaaattccattagctgggaatccagtctcccctacttcctgagaagactccaactttgaaataaacctgcgactgccattcaggatattgtctttgtagaaaatgatataaatttaagatgctcccagcaaagaagaaagaacgaggaagtgatcctggatgagaaatgctgtgccaggcatctttaagctctgcccttactctgtgtgggtttaagaaataaacgttcatttg

SEQ ID NO: 14 - DNA, 433 bp, Mus musculus
  misc_feature (94)..(94): Unknown
actgtgtaggtttccaaggctatgaggaatatatttcaggcagatttttcaatgataaccccaaatatttgtccatgattgtggcttcaagaanttttccagttacttgataaaggcctagtcatccagtaaggacccagtgtgtctagcatagtcacatttagctttcagaatacagtccttcactgctgagcagaatccagttcccctactgtgttaaattgctctatgaaacttcagtctaagtcccccaagccatagggagaatgaagaaggtgcaccttccagaagtctagcccctggactaacaatgactggagtgggtgcgtgcacttcagatctatgaaacatttaatgctttttgccccgtgaaaaatccattcctggtaaaagggaggcactgtttatataaacccaggagcaaaatgaactg

SEQ ID NO: 15 - DNA, 580 bp, Mus musculus
  misc_feature (409)..(409): Unknown
acgcgggggagccacacaaactcagggaaagctcgaatatggttttcaaacctcagatacttggacttatgctttttaggatttcagcctccagaggtgatattggctacctcaatctccagccaccctgtctgtgcctccaggagatagcgtcagtctttccttcagggccagccaaaatattagcaacaacctacactgggttcaaaaaaaccacatgagtctccaaggcttctcattaaagtcacacctggcctcatcacgttcggagctgggaccaagctggagctgaaacggactgatgctgcaccaactgtatccaatcttcccaccatccagtgaacagttaacatctggaggtgcctcagtcgtgtgcttcttgaacaacttctaccccaaagacatcaantgtcaaagtgggaagwtttgatgggcagatgaaaccgaacaaaaatggcgtccctggaacagtttgggaactgatcagggacaggcaaagaacagcacctacagcatggaagcagcaacccttcaacgtttgaccaaggaccgaagttattgaaaccgaccataacagctt

SEQ ID NO: 16 - DNA, 356 bp, Mus musculus
  misc_feature (1)..(2): Unknown
nngnnccctcacgttgaccaagggnngagtatgaaggacataacagctatacctgtgaggccactcgcaaggcatcaacttcacccattgtcaagaggttcaacaggaatgagtgttagagacaaaggtcctgagacgccaccaccagctccccagccccatcctattttcccttttaaggtcttggaggttccccccaagcgacctaccactgttggggtgctccaaacctcctccccacctccttttcctcctcctccctttccttggcttttatcatgctaatatttgcagaaaatattccaataaagtgagtctttgcccttaaaaaaaaaaaaaaaaaaaaaaaaaaaaaa

SEQ ID NO: 17 - DNA, 820 bp, Mus musculus
  misc_feature (145)..(145): Unknown
acaaattggatgcaaaatccaaaattcgagccctccttcgtctccatcaggagtgtgaaaagttgaaaaagctcatgagttctaacagcacggacctgccgctgaacatcgagtgctttatgaatgacaaggatgtctctgggangatgaacaggtcacagtttgaagaactgtgtgctgagctcctgcaaaaaatagaggtcccccttcactcgttgatggcacagactcagctcaaggctgaanatgtgagtgccattganatagtgggaggtgcccaagaatcccanctgtnaaagaaagaattgccaanttctttggaaaagatgtnancacccacnctnaatgntgacnaagctgtaggccngnaggctgangcactgnctngtncaatnctttctccggcattttaaagttacagantcntnttttcncnccatgctacccnttntccnaatatcanctggtcttggaacccnntgnctcnnnangaaaacnttaacnntgttcnccnaccttntntcatnncnggaaccntgnncgntnnttttnttccanacgtgtcnnnncctttnctnngcanaggggnggccctttngtnctacnaacnnttttcttcttnaccncncnanggnantncnngtnnntaaancaanacnannnnnccnnatannactgccnnaacntttntaccntcnanccgntnnttttcccnnncnctanngtnanancctcnaannnncntngtntnttcatcntncnncnccntnncttgcnnnnangnccnnncacnnccannnnactnctcttcctannctcn

SEQ ID NO: 18 - DNA, 842 bp, Mus musculus
  misc_feature (9)..(9): Unknown
acgcggggnattgtcattgcagtcaggnactcagcatggnacatgagggctcctgcacagnatttttggcttcttgttgctcttgtttccaggtaccagatgtgacatccagatgacccagtctccatcctccttatctgcctctctgggagaaagagtcagtctcacttgtcgggcaagtcaggatattggtagtagcttaaactggcttcagcaggaaccagatggaactattaaacgcctgatctacgccacatccagtttagattctggtgtccccaagaggttcagtggcagtaggtctgggtcagattattctctcaccatcagcagccttgagtctgaagattttgtagactattactgtctacaatatgctacttctcctccgacgttcggtgggggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatgggcgtcctgaacagttggactgatcaggacagcaaaanacngcacctacagcatgancagcacccttacgttgaccaaggacnagtttgaacgacntancnctttacctgngaggccctnacaaaaatnacttncccattgnnagacttcacaggaatgagtgtanaaacaaggnctgnacnccccccnntnccanttcntctatttcctttaaggttggaggttccccannacnccnttntgggggctcaaactntnccnctcttt

SEQ ID NO: 19 - DNA, 603 bp, Mus musculus
  misc_feature (299)..(299): Unknown
acttgaactggccttcttatccaggataagtggagttcatgcaaggtggatccctcatgataggattagtggctttataagaacagtgattgagaaagaattcattctgttgacgcccatgcaagcggaacagcaaactgcttaagaactcatggaatgtggcccactggttagcaccatctcaatgggtgcgccttggagaggcactacatgtcctgaggatcacatgctgctgtagagcgtcactctgcttgttacccttgtggtggtcatcatgtctccccctaatctatgagctngtatgaaaactctaagagggtttccagaccctcacagggcagtgtcattggcatttacaatagttgttcttgatctttctcaagcattactgctggggttagattaatgaattcagtttaccattcaantaaagttagggttaaggtatttttgctaagtnggtttntgatcgtaccaaaatcttggaggagcaatggtcaaagtttttagaaaggccntttangaaaagttttgataacccnantggaaaggattnttttggangntcnagggnaancaaagnanttaaccaccaagttncnt

SEQ ID NO: 20 - DNA, 355 bp, Mus musculus
ctttttaagataaattgaaacctgccagtaagacaacctggggagaaaattgccttgttaagttataacaagttctttggttatgaatgcatgtgggttcttgggaataatttgtttttaacccataatatgtaaaacatttgatcatgtgaagctattgaggagcatatgtatctctcttttttaaaaatgtattctcattgtaattattcacttaaagaaaagtagaatgtaaccacatcgtggttttatgctgcttgaaagaatttcctggtagatatataagctatggaagaaaaataaaaattttggagcttgcatcatccgaaaaaaaaaaaaaaaaaaaaaaaaaagt

SEQ ID NO: 21 - DNA, 580 bp, Mus musculus; identical to SEQ ID NO: 15
  misc_feature (409)..(409): Unknown

SEQ ID NO: 22 - DNA, 356 bp, Mus musculus; identical to SEQ ID NO: 16
  misc_feature (1)..(2): Unknown

SEQ ID NO: 23 - DNA, 687 bp, Mus musculus
  misc_feature (128)..(128): Unknown
acctccccaaaatgactcttttggagcacagttacctccaatgcatcagcagcaaagcagatctgtaatgacagaagaatacaaagttccagatggaatggttggatttataattggcagaggaggtnaacagatctcacngngngcagcaggaatctggatgcangatacanatagcncctgatagtggtggcctaccanaaaggtctngtatgctaactggaacacctgaatctgtccagtcagcaaaaagattattggaccagattgttgaaaagggaagaccagcccctggccttcatcatggngatggacctggaaatgcagttcaggaaatcatgattccanccagcnaagcnaggactagnttattcggaaaggggggccgagactattaatcnnctttcncgaacggcctgggtgttaaaatggttatgattnaagatnggcctcaaaaaccntgggggtgatnaacctcttagggattnggggttnnccatacaaagttnagcaagccaagcnnnangtattaggagttaattngtgaanaaggtggtttcagagaagtgcggaatgagtatggctcaanaataggaggcaatgaagggatagatgtcccaatttccaagatttgctgttggcattgtaataggaagttntttnaaaaanaacnnccaaaaaaaaaaa

SEQ ID NO: 24 - DNA, 975 bp, Mus musculus
  misc_feature (3)..(4): Unknown
ttnngnantacngngcncaacccnttttaggaacggnccccantgtgctggaattcgcccttagcttggtcgcggccgaggnnctacggctcaaaaccccgtcaccacggcccggatcgggaagtgtcaagtgggagccgggttcccccgcatttgcagccgccacanctgaacccggacccctcaccccagccgggcnctaccgccntttttaacntgtnctnnngngggngggatcctggagttngntttgataaaaaaanccccccctgtgccngngggnagnaccnncnntntncctccaaaccnngnctgctnccaataaaananaggncntcgtggnnttntatacatacaaaancnccctnatnatcngnaacnacnaccnnnntncctnnctngttcnantancccncancngacaaatcnccctntncacatncccccnccnaacnnnctacaantcnacaaacccctnncnacccccattnatcttagcccncccntaggncnancntacaccnanccaatcnngcacancnaanacccngccnccccctccnctacnaacctccnnacctntcatacnttccnnccgtcncccnntnatcaacnnctccnntacnncnctcccccccccnannngttcccccaaancnnnccactaancacccgnacctttnnnncccntnaanagnccatcccctaantncccaccccctttcnctcnntnactntntnnnnncccccncacacancantntacnnntnttctntncaccacaaccatttccccnantttnacntccaatctcantcctacccccnncnacacccncacctcacttcaaccatanaccnnncncnttcgtccccancncaccncannncccctnncncacncactcttcnnntcnntncnnnttcacccantaccccntcgcaacgntcccncnantnccannntcccncacn

SEQ ID NO: 25 - DNA, 42 bp, Mus musculus
  misc_feature (5)..(5): Unknown
gaagngnaaaattttttttaaaaaaaaaaaaaaaaaaaaaaa

SEQ ID NO: 26 - DNA, 318 bp, Mus musculus
  misc_feature (2)..(2): Unknown
gntctgtgaaanganatagannggctcgaaggaccaaggcancttcctgactgaagaccagatcctgccgctgttgctgggtatcagcagaggccttgaggctattcatgccaaaggttatgcacacagggacctgaagcccgtngnnnattttgcttggtgatgaggggcanccaantntaatggacatgggttntnttaatcanttcatgcattcaagtggagggctctcgccaggcactaactcttcaggactgggcagctcagcggtgcaccatctcctaccgggcacctgaacttttttctgtgcaaagcctn

SEQ ID NO: 27 - DNA, 860 bp, Mus musculus
  misc_feature (5)..(5): Unknown
ctagnggcctttgtttttnntnaggtttcccggggggtggcnccctttttntttntngggccccccttttttgnantggntntttttttncnncgccctctgggggggnntcncgcggcgcntttttttgttgngggggntttgggggngncnntttcttcgtgggnccgntnttttttttntncnaaaccccnngnttcnttnttgggggctntttgtgnnncgnngggggnngntttttcccttntttttngggggnggccccncnntattgnngtttnccaanngggggcganngngcccntcttgggccaaattnagngngcntcccccncannccccccggctttttttancnttgtgnanntttttntttttttnttgtgggggggggccccccgcgggggnctncntnccctntncnncncngtnaggntgggggnntttccccccccngnttnnggccgnncccccccccnntttnnntnnannnngngggcnttttggggnangncccntnnaggggntttttncccccnttgggncncnnnccntttttnccgggnggntttnccngntnnnggcanggnnagggggnccntnttttttttccctttttccccccantcnngggnnngggccctngggggggnnggggggaantttanccnnnantttgggncnaanntngggnngggggggcccccnncnttttntttcccntntngggggnnaaaaaaancntnaaaaggnnaatnangttaaacccncccaaaaaaaaaaaaaaaaaaaaaaaaaaaaaaaaaaaaaaaaaaaaaaaaaaaaaaaaaaaaaaaaaaaaaaaaaaaaaaacaaaaaaa

SEQ ID NO: 28 - DNA, 935 bp, Mus musculus
acgcggggtttccggcttgaggatccgtcgcgtttctgaggcggcgcctcagtcggcagttaggatcggctcggagcggtgggtctcggccccggcgtcaccatgggggccgtcctcggcgtcttctccctcgccagctgggtcccgtgcctctgtagtggtgcatcatgtctgctgtgcagttgctgtcccatcagtaagaattccactgtaactcgggtcatctacgcttttatcctcttccttggcactattgtgtcttgcatcatgatgacagaaggcatacaaactcaactgaagaagattcctggattctgtgaaggaggatttcaaatcaagatggttgatacaaaggcagagaaagattgtgacgtgctggtcggttttaaagctgtgtatcggatcaactttgctgtggccatttttttctttgccttctttttgctcatgttaaaagttaaaacaagtaaagatcccagagcagcagtgcacaacgggttttgggttcttcaaaatcgctgccattatggtatcatgattggatctttctacatccctggggggagttttactgaagtctggttttttgctggaatgttgggggcctctttcttcattatcatccagctggtgctcttggtagacatggctcactcttggaatgaattatgggtaaatcgaatggaggaaggaaacccaaggctctggtatgctgccttgctgtcctttacaagcctcttttacatcctctccatcgtctttgccgcgctgctctacgtcttttacaccaagcctaacgactgcacagaaaacaaggtcttcatcagcctcaacctgattttttgtgttgcagtttcatattgtgtccatcctccgtaaagttcaggaacatcagcctcgctctggcctcctgcagtcctccatcattactaaga

SEQ ID NO: 29 - DNA, 535 bp, Mus musculus
acgcggggatctaggaggctgctgacctccaacaggaatttcaccacttaaccctctagaagtcccactacttaatacttcagtacttctagcatcaggtgtttcaattacatgagctcatcatagccttatagaaggtaaacgaaaccacataaatcaagccctactaattaccattatactaggactttacttcaccatcctccaagcttcagaatactttgaaacatcattctccatttcagatggtatctatggttctacattcttcatggctactggattccatggactccatgtaattattggatcaacattccttattgtttgcctactacgacaactaaaatttcacttcacatcaaaacatcacttcggatttgaagccgcagcatgatactgacattttgtagacgtaatcttgacttttcctatacgtctccatttattgatgaggatcttaaaaaaaaaaaaaaaaaaaaaaaaaaaaaaaaaaaaaaagaaaaaaaaaaaaaaaaaaaaaaaaaaaaaaaaa

SEQ ID NO: 30 - DNA, 1171 bp, Mus musculus
  misc_feature (9)..(9): Unknown
acgcggggnagttgcggccctgtgcgcttccacagcgtcccagcctttgtcctggcctcagctcctgcttcctgacctgcttgagttccagtcctgacttcctttggcgataaacagcagtatggaaatacctcacagtggatgttcctggccatggaaaggtgctggtggacattgcatatggtggggcattttatgcgtttgttagtgcagaaaaattaggacttgatgtgtgttctgcgaagacgagggaccttgtggatgcggcaagtgcattgacaggagcagtgaaagcacagtttaaaatcaaccatcctgagagtgaagaccttggttttttgtatggatccatcttgacggatggaaaagatgcttatagtgaggaggccaccaccaacatctgcgtgtttgccgatgaacaggttgacagaagccccaccggctcgggagtgacagccagaattgctctgcagtatcataaggggctcctgcagctgaaccagaccagagccttcaaaagcagcgcaactggctcggtgttcacaggctgtgccgtgagggaagcaaagtgtggagatttcaaagctgtcatagtggaagttgcaggacaagcccacttacncagggrcagcaaacttgmcagtggaagatggtgaccccctaagggatggcttttttttcaagkgacttttttttcataagttttaaaggntttgcttanaaagaaatwccycttaaatggggntttntctgnattatgggcnaaccngtntccngttacccttaccgggtaatttttanactttttaaaaagtatgtttagtanactgngtttaaatgtaaggnnttatacttcctntttgtcacccntttgngcataaatgtngntaatggggcnaacanagngccaanaatccggaaacttttnaagccttngtntncaaggcnaaattnaagcccngttncttaatttntgctgnggaaatgataaaacttgtaagttttttttgattgntagnttcagctntnanccccccnatagtaaatgttgngngtttgttnttttaatgttccngtttgngtaaaataaaggcntcntgtaagnttccaaaaaaaaaaaaaaaaaaaaaaaaaaaaaaaaaaancaaaaaaaaaaaaaaaaaaaaaaaaa

SEQ ID NO: 31 - DNA, 932 bp, Mus musculus
acgcggggagactggggaaatcgaaattatttcagcagctgtcttcagtcactgggtgtgtcttcctgcacagtggacttgacgggcctggagggaggagaagaaattgaaacctatctcatcctctagtttatttacatcattggattcggttcctgtttggaaacttaactggtcctcatggcgttttcgggcacagggacactggtggcttccattgtctccaagatgacatcttcagcagccatggtcaaagctggaggggcagtctccggcaccattctagctggttcgaaggggctcatcctgttaacacagagtgccctgggctctgcaacatctgcacttggagcaccgaaggttggcaccattttatctgggttctctgcctccaccttggctgcttcccccattggagccaaggctgttgtggctgtgcttgggggagccatgacagtagctgctgtaccacctgcgctgagtgctgtgggcttcaccgcctcaggaattgcagcctcctctctagcagctaagatgatgtccttgtcagctattgctaatgggggtggagtcccagctggtggcctggtggccattttgcagtctgctggagctgctggactttccgtgccatctactgtcatcgtgggctttgcagggtttgccgttgtggccagtgtcatgaacatctgtgaaagtttttacccttttttcatgggatcagaggtggcagacatggcgacaaaggtggcagatatggcgataaaggtggcagacatttcaacaaaaaaggcactgcccaacttgtttaacccagagaaggattaattacccccaggcgacaatttcctaacacatattgcctaggtggaaataaattaaaccgagtttttaaaaaaaaaaaaaaaaaaaaaaaaaaaaaaaaaaaaaaa

SEQ ID NO: 32 - DNA, 890 bp, Mus musculus
  misc_feature (9)..(9): Unknown
accaccggnaccacagtncagcaacgtcaagnatggacatcctgtcttacatgnaggccggcaggcagccctgagcgccagggggaagtaactgatgcccccaccctacccctaccccctgcccatcatgcaagagccgaggaggggctcttcaggaaggaagccacattcccagtcattctacccccaccccagattctctttcttattacataaaagacaagcctggcacaactgtgtgtctgaaccactgtggacacgtgacaattgtcccagtgtgtgcatggctacacagccacgtatctgcctgcttgaaacccagggatggtccatctgtgtttacggcttggcacaacaccctcatatttttttcagctttctgttccaaatgagcccaaaggaaacacaagttctaggtccaatggttctgctcaaacctgaacatcattcttggggccagcatctcccacacgcccacactacacaccaccagcctccttcttccttcctgaaggaccctcctgagcccccaagcccatcccacagtgctcctgagaccagccaagacaactgtgagcgcgatggccgtgtaccttgcccgggcggccgtcgaaagggcgaattccagccacttggcggccgtactagtggatccgagctcggtccaacttgatgcatacttggantnttntatagtggcacctaaatagcttggngtaatntgggnatagctgnttctgngngaaatggtnttccgttanantccnacaacnacaacccgnacataagtgtaaanctggggngcnangangncnnactanttantnnntngnctnntgccnttcannngnaactnnnncnntnntnnantgcaccccggnnggngntnn

SEQ ID NO: 33 - DNA, 878 bp, Mus musculus
  misc_feature (549)..(549): Unknown
accacggggacaggatttgggacgttcctgatgaccgtgttgctgaaatacctgtgcgcagaatatggctggcggaatgctatgttcatccaaggtgctctgtccctgaacctgtgtgtctgtggggcgctcatgaggcccctctctcccgagaagttagaaaactgcccagaagcggaagagccgtgtgctctcccagcttactccactgaatctgttaagtctgggggaccactgggcatggctgaagaacaggacagaaggcccgggaatgaggagatggtgtgtgaccttcaaacacaggagtgtcaaggtcaaacccatccgaggaagaatgtgtgtgccttccgggttctgaagacagtgagccagctcactgtgcaagtccggaggggctttagggactggcactcaggctattttgggacggcttcactcttcaccaaccgcatgtttgtagcctttattttctgggctctgttcgcatacagcagctttgtcatccctttcatccacctgcccgaaatcgtcagtttgtataacttgtcngaaccaaaatgacacattccctcttgacctcgattatagcaatacttcacatcttcgggaaggtgatctttgggggccgtggnccacctcccttgtatcancgtctggaatggtcttcctnatnngttacttnaccctcgtcctnnnatnttnctttttgcctttgatgcncacctccccaanntggntnnctttgggccctaatgggntttctagcggttttntcctnnncctggggnacnangactgntngcntnnaccttngcnatgctacgnntntattnnccanggntttnncttntngancccntnnngaggatttncnccca

SEQ ID NO: 34 - DNA, 674 bp, Mus musculus
acttgctggtggtccatccctgcactatttccaagccaggtcttgggatcagtgacttgcttcctgcagtgctgtggtcacaggcccctccagtcagccatgtctagcagcttggcaggcttaggggctcggtgtgcagatttctttttgtgcagcagaatgccaagagactgagcagagagtcttcttaaaacctaccctaagggccgccaggtccatgtgcccgtggtaggtctggggccaggcagctgctgctgtcctgatcccccctcccttagctatggatccttgtagtagaggtcagccgctcctggcgcaaacaggatccagtagctgtctgccacccacgaggtttctaagataccatgtggagacagggtcctccccatccttttcagaaatagcagcttgttttctctgctggcatgtgtccagggcaccaggtcccagcagggggaggcagactgttaactgaacscgcataggaacaggaggcagcgagagcctgtgggaggaagggccttctagtgaccctgcttctccatcctgcctccctcctgtccacccagtttcagagttgggaggggaggagccatggctccctcacccaggcttggtggctgtggccctgttttagagccatggcagcacaggctggtgagtggtagagttgt